(19) United States
(12) Patent Application Publication
(10) Pub. No.: US 2002/0041880 A1
(43) Pub. Date: Apr. 11, 2002

(54) Method of treating cancer

(76) Inventors: Deborah DeFeo-Jones, Lansdale, PA (US); David C. Heimbrook, Coopersburg, PA (US); Raymond E. Jones, Lansdale, PA (US)

Correspondence Address: MERCK AND CO INC, P O BOX 2000, RAHWAY, NJ, 070650907

(21) Appl. No.: 09/896,251

(22) Filed: Jun. 29, 2001

Related U.S. Application Data
(60) Provisional application No. 60/215,934, filed on Jul. 5, 2000.

(51) Int. Cl. A61K 39/00; A61K 38/14; A61K 38/08

(52) U.S. Cl. 424/185.1; 514/17; 514/8

(57) ABSTRACT

The present invention relates to methods of treating cancer using a combination of a compound which is a PSA conjugate and a compound which is a inhibitor of angiogenesis, which methods comprise administering to said mammal, either sequentially in any order or simultaneously, amounts of at least two therapeutic agents selected from a group consisting of a compound which is a PSA conjugate and a compound which is a inhibitor of angiogenesis. The invention also relates to methods of preparing such compositions.

### RELATED APPLICATION

The present patent application claims the benefit of provisional application Ser. No. 60/215,934, filed Jul. 5, 2000, which was pending on the date of the filing of the present invention.

### BACKGROUND OF THE INVENTION

The present invention relates to methods of treating cancer, and more particularly cancer associated with cells that produce prostate specific antigen (PSA), which comprise administering to a patient in need thereof at least one inhibitor of angiogenesis and at least one conjugate, which comprises an oligopeptide that is selectively cleaved by PSA and a cytotoxic agent.

In 1999 new cases of cancer of the prostate gland were expected to be diagnosed in 179,300 men in the U.S. and 37,000 American males were expected to die from this disease (Landis, S. H. et al. CA Cancer J. Clin. 49:8-31 (1999)). Prostate cancer is the most frequently diagnosed malignancy (other than that of the skin) in U.S. men and the second leading cause of cancer-related deaths (behind lung cancer) in that group.

Prostate specific antigen (PSA) is a single chain 33 kDa glycoprotein that is produced almost exclusively by the human prostate epithelium and occurs at levels of 0.5 to 2.0 mg/ml in human seminal fluid (Nadji, M., Taber, S. Z., Castro, A., et al. (1981) Cancer 48:1229; Papsidero, L., Kuriyama, M., Wang, M., et al. (1981). JNCI 66:37; Qui, S. D., Young, C. Y. F., Bihartz, D. L., et al. (1990), J. Urol. 144:1550; Wang, M. C., Valenzuela, L. A., Murphy, G. P., et al. (1979). Invest. Urol. 17:159). PSA is a protease with chymotrypsin-like specificity (Christensson, A., Laurell, C. B., Lilja, H. (1990). Eur. J. Biochem. 194:755-763). It has been shown that PSA is mainly responsible for dissolution of the gel structure formed at ejaculation by proteolysis of the major proteins in the sperm entrapping gel, Semenogelin I and Semenogelin II, and fibronectin (Lilja, H. (1985). J. Clin. Invest. 76:1899; Lilja, H., Oldbring, J., Rannevik, G., et al. (1987). J. Clin. Invest. 80:281; McGee, R. S., Herr, J. C. (1988). Biol. Reprod. 39:499). The PSA mediated proteolysis of the gel-forming proteins generates several soluble Semenogelin I and Semenogelin II fragments and soluble fibronectin fragments with liquefaction of the ejaculate and release of progressively motile spermatozoa (Lilja, H., Laurell, C. B. (1984). Scand. J. Clin. Lab. Invest. 44:447; McGee, R. S., Herr, J. C. (1987). Biol. Reprod. 37:431). Furthermore, PSA may proteolytically degrade IGFBP-3 (insulin-like growth factor binding protein 3) allowing IGF to stimulate specifically the growth of PSA secreting cells (Cohen et al., (1992) J. Clin. Endo. & Meta. 75:1046-1053).

PSA complexed to alpha 1-antichymotrypsin is the predominant molecular form of serum PSA and may account for up to 95% of the detected serum PSA (Christensson, A., B\ddot{j}ork, T., Nilsson, O., et al. (1993). J. Urol. 150:100-105; Lilja, H., Christensson, A., Dahlén, U. (1991). Clin. Chem. 37:1618-1625; Stenman, U. H., Leinoven, J., Alfthan, H., et al. (1991). Cancer Res. 51:222-226). The prostatic tissue (normal, benign hyperplastic, or malignant tissue) is implicated to predominantly release the mature, enzymatically active form of PSA, as this form is required for complex formation with alpha 1-antichymotrypsin (Mast, A. E., Enghild, J. J., Pizzo, S. V., et al. (1991). Biochemistry 30:1723-1730; Perlmutter, D. H., Glover, G. I., Rivetna, M., et al. (1990). Proc. Natl. Acad. Sci. USA 87:3753-3757). Therefore, in the microenvironment of prostatic PSA secreting cells the PSA is believed to be processed and secreted in its mature enzymatically active form not complexed to any inhibitory molecule. PSA also forms stable complexes with alpha 2-macroglobulin, but as this results in encapsulation of PSA and complete loss of the PSA epitopes, the in vivo significance of this complex formation is unclear. A free, noncomplexed form of PSA constitutes a minor fraction of the serum PSA (Christensson, A., Björk, T., Nilsson, O., et al. (1993). J. Urol. 150:100-105; Lilja, H., Christensson, A., Dahlén, U. (1991). Clin. Chem. 37:1618-1625). The size of this form of serum PSA is similar to that of PSA in seminal fluid (Lilja, H., Christensson, A., Dahlén, U. (1991). Clin. Chem. 37:1618-1625) but it is yet unknown as to whether the free form of serum PSA may be a zymogen; an internally cleaved, inactive form of mature PSA; or PSA manifesting enzyme activity. However, it seems unlikely that the free form of serum PSA manifests enzyme activity, since there is considerable (100 to 1000 fold) molar excess of both unreacted alpha 1-antichymotrypsin and alpha 2-macroglobulin in serum as compared with the detected serum levels of the free 33 kDa form of PSA (Christensson, A., Björk, T., Nilsson, O., et al. (1993). J. Urol. 150:100-105; Lilja, H., Christensson, A., Dahlén, U. (1991). Clin. Chem. 37:1618-1625).

Serum measurements of PSA are useful for monitoring the treatment of adenocarcinoma of the prostate (Duffy, M. S. (1989). Ann. Clin. Biochem. 26:379-387; Brawer, M. K. and Lange, P. H. (1989). Urol. Suppl. 5:11-16; Hara, M. and Kimura, H. (1989). J. Lab. Clin. Med. 113:541-548), although above normal serum concentrations of PSA have also been reported in benign prostatic hyperplasia and subsequent to surgical trauma of the prostate (Lilja, H., Christensson, A., Dahlén, U. (1991). Clin. Chem. 37:1618-1625). Prostate metastases are also known to secrete immunologically reactive PSA since serum PSA is detectable at high levels in prostatectomized patients showing widespread metatstatic prostate cancer (Ford, T. F., Butcher, D. N., Masters, R. W., et al. (1985). Brit. J. Urology 57:50-55). Therefore, a cytotoxic compound that could be activated by the proteolytic activity of PSA should be prostate cell specific as well as specific for PSA secreting prostate metastases.

Conjugates which comprise an oligopeptide which can be selectively cleaved by enzymatically active PSA attached, either directly or via a linker to a cytotoxic agent and which are useful in the treatment of prostate cancer and benign prostatic hyperplasia have been previously described (U.S. Pat. Nos. 5,599,686 and 5,866,679).

Several lines of direct evidence now suggest that angiogenesis is essential for the growth and persistence of solid tumors and their metastases (Folkman, 1989; Hori et al., 1991; Kim et al., 1993; Millauer et al., 1994).

Once tumor ‘take’ has occurred, every increase in tumor cell population must be preceded by an increase in new capillaries converging on the tumor. Tumor ‘take’ is currently understood to indicate a prevascular phase of tumor growth in which a population of tumor cells occupying a few cubic millimeters volume and not exceeding a few million cells, can survive on existing host microvessels. Expansion of tumor volume beyond this phase requires the induction of new capillary blood vessels.

Angiogenesis begins with the erosion of the basement membrane by enzymes released by endothelial cells and leukocytes. The endothelial cells, which line the lumen of blood vessels, then protrude through the basement membrane. Angiogenic stimulants induce the endothelial cells to migrate through the eroded basement membrane. The migrating cells form a “sprout” off the parent blood vessel, where the endothelial cells undergo mitosis and proliferate. The endothelial sprouts merge with each other to form capillary loops, creating the new blood vessel.

To stimulate angiogenesis, tumors upregulate their production of a variety of angiogenic factors, including the fibroblast growth factors (FGF and BFGF) (Kandel et al., 1991) and vascular endothelial cell growth factor/vascular permeability factor (VEGF/VPF). Vascular endothelial growth factor (VEGF) binds the high affinity membrane-spanning tyrosine kinase receptors KDR and Flt-1. Cell culture and gene knockout experiments indicate that each receptor contributes to different aspects of angiogenesis. KDR mediates the mitogenic function of VEGF whereas Flt-1 appears to modulate non-mitogenic functions such as those associated with cellular adhesion. Inhibiting KDR thus modulates the level of mitogenic VEGF activity.

Expression of VEGF is also significantly increased in hypoxic regions of animal and human tumors adjacent to areas of necrosis. VEGF is also upregulated by the expression of the oncogenes ras, raf, src and mutant p53 (all of which are relevant to targeting cancer). Monoclonal anti-VEGF antibodies inhibit the growth of human tumors in nude mice. Although these same tumor cells continue to express VEGF in culture, the antibodies do not diminish their mitotic rate. Thus tumor-derived VEGF does not function as an autocrine mitogenic factor. Therefore, VEGF contributes to tumor growth in vivo by promoting angiogenesis through its paracrine vascular endothelial cell chemotactic and mitogenic activities. These monoclonal antibodies also inhibit the growth of typically less well vascularized human colon cancers in athymic mice and decrease the number of tumors arising from inoculated cells. Viral expression of a VEGF-binding construct of Flk-1, Flt-1, the mouse KDR receptor homologue, truncated to eliminate the cytoplasmic tyrosine kinase domains but retaining a membrane anchor, virtually abolishes the growth of a transplantable glioblastoma in mice presumably by the dominant negative mechanism of heterodimer formation with membrane spanning endothelial cell VEGF receptors. Embryonic stem cells, which normally grow as solid tumors in nude mice, do not produce detectable tumors if both VEGF alleles are knocked out. Taken together, these data indicate the role of VEGF in the growth of solid tumors. Inhibition of KDR or Flt-1 is implicated in pathological neoangiogenesis, and these receptors are useful in the treatment of diseases in which neoangiogenesis is part of the overall pathology, e.g., inflammation, diabetic retinal vascularization, as well as various forms of cancer. The compounds of the instant invention represent novel structures for the inhibition of KDR kinase.

Numerous classes of compounds have been described as inhibitors of angiogenesis.

It is the object of the instant invention to provide a method for treating cancer, and more particularly cancer associated with cells that produce prostate specific antigen (PSA), which offers advantages over previously disclosed methods of treatment.

### SUMMARY OF THE INVENTION

A method of treating cancer, and more particularly cancer associated with cells that produce prostate specific antigen (PSA), is disclosed which is comprised of administering to a patient in need of such treatment amounts of at least one inhibitor of angiogenesis and at least one conjugate, which comprises an oligopeptide that is selectively cleaved by PSA and a cytotoxic agent.

### DETAILED DESCRIPTION OF THE INVENTION

The present invention relates to a method of treating cancer, and more particularly cancer associated with cells that produce prostate specific antigen (PSA), which is comprised of administering to a patient in need of such treatment amounts of at least one inhibitor of angiogenesis and at least one conjugate (hereinafter referred to as a PSA conjugate), which comprises an oligopeptide that is selectively cleaved by PSA and a cytotoxic agent. Such a combination of an inhibitor of angiogenesis and a PSA conjugate may also be useful in treating prostatic diseases in general, including prostatic cancer, benign prostatic hyperplasia and prostatic intraepithelial neoplasia.

In practicing the instant method of treatment, it is understood that the inhibitor(s) of angiogenesis and the PSA conjugate(s) may be administered either simultaneously in a single pharmaceutical composition or individually in separate pharmaceutical compositions. If the inhibitor(s) of angiogenesis and the PSA conjugate(s) are administered in separate compositions, such compositions may be administered simultaneously or consecutively.

The term “consecutively” when used in the context of administration of two or more separate pharmaceutical compositions means that administrations of the separate pharmaceutical compositions are at separate times. The term “consecutively” also includes administration of two or more separate pharmaceutical compositions wherein administration of one or more pharmaceutical compositions is a continuous administration over a prolonged period of time and wherein administration of another of the compositions occur at a discrete time during the prolonged period.

The terms angiogenesis inhibitor and inhibitor of angiogenesis refer to compounds which inhibit or eliminate the formation of and proliferation of new blood vessels in the vicinity of and within the tumor. Such inhibitors may inhibit angiogenesis by one of a number of mechanisms. For example, the angiogenesis inhibitor may block the initial breakdown of the vascular matrix by inhibiting matrix metalloproteinases, may inhibit the growth of endothelial cells, or may block the activators of angiogenesis: factors such as fibroblast growth factors, vascular endothelial growth factor and vascular permeability factors.

The angiogenesis inhibitor may alternatively inhibit endothelial-specific integrin/survival signaling.

The instant method of treatment also comprises a PSA conjugate. The PSA conjugate comprises an oligopeptide, which is specifically recognized by the free prostate specific antigen (PSA) and are capable of being proteolytically cleaved by the enzymatic activity of the free prostate specific antigen, covalently bonded directly, or through a chemical linker, to a cytotoxic agent. Ideally, the cytotoxic activity of the cytotoxic agent is greatly reduced or absent when the oligopeptide containing the PSA proteolytic cleavage site is bonded directly, or through a chemical linker, to the cytotoxic agent and is intact. Also ideally, the cytotoxic activity of the cytotoxic agent increases significantly or returns to the activity of the unmodified cytotoxic agent upon proteolytic cleavage of the attached oligopeptide at the cleavage site. While it is not necessary for practicing this aspect of the invention, a preferred embodiment of this aspect of the invention is a conjugate wherein the oligopeptide, and the chemical linker if present, are detached from the cytotoxic agent by the proteolytic activity of the free PSA and any other native proteolytic enzymes present in the tissue proximity, thereby releasing unmodified cytotoxic agent into the physiological environment at the place of proteolytic cleavage. Pharmaceutically acceptable salts of the conjugates are also included.

Oligopeptides that are selectively cleaved by enzymatically active PSA can be identified by a number of assays, in particularly the assays described in the Biological Assays of the Examples.

In one embodiment of the instant invention, the oligopeptide component of the PSA conjugate incorporates a cyclic amino acid having a hydrophilic substituent as part of the oligopeptides, said cyclic amino acid which contributes to the aqueous solubility of the conjugate. Examples of such hydrophilic cyclic amino acids include but are not limited to hydroxylated, polyhydroxylated and alkoxylated proline and pipecolic acid moieties.

In a prefered embodiment of the invention the oligopeptide component of the PSA conjugate is characterized by having a protecting group on the terminus amino acid moiety that is not attached to the cytotoxic agent. Such protection of the terminal amino acid reduces or eliminates the enzymatic degradation of such peptidyl therapeutic agents by the action of exogenous aminopeptidases and carboxypeptidases which are present in the blood plasma of warm blooded animals. Examples of protecting groups that may be attached to the amino moiety of an N-terminus oligopeptide include, but are not limited to acetyl, benzoyl, pivaloyl, succinyl, glutaryl, hydoxyalkanoyl, polyhydroxyalkanoyl, polyethylene glycol (PEG) containing alkanoyl and the like. Examples of protecting groups that may be attached to the carboxylic acid of a C-terminus oligopeptide include, but are not limited to, formation of an organic or inorganic ester of the carboxylic acid, such as an alkyl, aralkyl, aryl, polyether ester, phosphoryl and sulfuryl, or conversion of the carboxylic acid moiety to a substituted or unsubstituted amide moiety. The N-terminus or C-terminus of the oligopeptide may also be substituted with a unnatural amino acid, such as β-alanine, or a D-amino acid, such as a D-valyl or D-alanyl group.

It is understood that the oligopeptide which is conjugated to the cytotoxic agent, whether through a direct covalent bond or through a chemical linker, does not need to be the oligopeptide that has the greatest recognition by free PSA and is most readily proteolytically cleaved by free PSA. Thus, the oligopeptide that is selected for incorporation in such conjugate will be chosen both for its selective, proteolytic cleavage by free PSA and for the cytotoxic activity of the cytotoxic agent-proteolytic residue conjugate (or, in what is felt to be an ideal situation, the unmodified cytotoxic agent) which results from such a cleavage.

Because the PSA conjugates useful in the instant compositions can be used for modifying a given biological response, the cytotoxic agent component of the PSA conjugate is not to be construed as limited to classical chemical therapeutic agents. For example, the cytotoxic agent may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“IL-1 ”), interleukin-2 (“IL-2”), interleukin-6 (“IL-6”), granulocyte macrophage colony stimulating factor (“GM-CSF”), granulocyte colony stimulating factor (“G-CSF”), or other growth factors.

The preferred cytotoxic agents include, in general, alkylating agents, antiproliferative agents, tubulin binding agents and the like. Preferred classes of cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of those classes include, for example, doxorubicin, carminomycin, daunorubicin, aminopterin, methotrexate, methopterin, dichloro-methotrexate, mitomycin C, porfiromycin, 5-fluorouracil, 6-mercaptopurine, cytosine arabinoside, podophyllotoxin, or podophyllotoxin derivatives such as etoposide or etoposide phosphate, melphalan, vinblastine, vincristine, leurosidine, vindesine, leurosine and the like. Other useful cytotoxic agents include estramustine, cisplatin and cyclophosphamide. One skilled in the art may make chemical modifications to the desired cytotoxic agent in order to make reactions of that compound more convenient for purposes of preparing PSA conjugates of the invention.

Preferably the cytotoxic agent component of the PSA conjugate is selected from a member of a class of cytotoxic agents selected from the vinca alkaloid drugs and the anthracyclines.

A pharmaceutical composition which is useful for the treatments of the instant invention may comprise one or more inhibitors of angiogenesis, one or more PSA conjugates, or a combination thereof, preferably, in combination with pharmaceutically acceptable carriers, excipients or diluents, according to standard pharmaceutical practice. The composition may be administered to mammals, preferably humans. The composition can be administered orally or parenterally, including the intravenous, intramuscular, intraperitoneal, subcutaneous, rectal and topical routes of administration.

The pharmaceutical compositions containing the active ingredients may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, cellulose acetate buryrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions useful in the instant methods of treatment may also be in the form of an oil-in-water emulsions. The oily phase may be a vegetable oil, for example olive oil or arachis oil, or a mineral oil, for example liquid paraffin or mixtures of these. Suitable emulsifying agents may be naturally- occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavouring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The instant compositions may also be administered in the form of suppositories for rectal administration of the drug. These compositions can be prepared by mixing the instant composition with a suitable non-irritating excipient which is solid at ordinary temperatures but liquid at the rectal temperature and will therefore melt in the rectum to release the composition. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the combination of inhibitor(s) of angiogenesis and PSA conjugate(s) are employed. (For purposes of this application, topical application shall include mouth washes and gargles.)

The compositions useful in the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

As used herein, the term “composition” is intended to encompass a product comprising the specified ingredients in the specific amounts, as well as any product which results, directly or indirectly, from combination of the specific ingredients in the specified amounts.

The composition of an angiogenesis inhibitor(s), a PSA conjugate(s), or a combination thereof useful in the instant methods of treatment may also be co-administered with other well known therapeutic agents that are selected for their particular usefulness against the condition that is being treated.

The instant method of treatment may also be combined with surgical treatment (such as surgical removal of tumor and/or prostatic tissue) where appropriate.

If formulated as a fixed dose, the compositions useful in the instant invention employ the angiogenesis inhibitor(s) and the PSA conjugate(s) within within the dosage ranges described below.

When compositions according to this invention are administered into a human subject, the daily dosage will normally be determined by the prescribing physician with the dosage generally varying according to the age, weight, and response of the individual patient, as well as the severity of the patient's symptoms.

In one exemplary application, a suitable amount of an inhibitor of angiogenesis and a suitable amount of a PSA conjugate are administered to a mammal undergoing treatment for prostate cancer. Administration occurs in an amount of inhibitor of angiogenesis of between about 2 mg/m² of body surface area to about 2 g/m² of body surface area per day, preferably between about 12 mg/m² of body surface area to about 1200 mg/m² of body surface area per day. A particular daily therapeutic dosage that comprises the instant composition includes from about 10 mg to about 3000 mg of an inhibitor of angiogenesis. Preferably, the daily dosage comprises from about 20 mg to about 2000 mg of an inhibitor of angiogenesis. A higher dosage of the inhibitor of angiogenesis may be administered if the inhibitor is administered in a single dose once a week. Administration of the PSA conjugate occurs in an amount between about 10 mg/m² of body surface area to about 5 g/m² of body surface area per day, preferably between about 50 mg/m² of body surface area to about 3 g/m² of body surface area per day.

Angiogenesis inhibitors that are inhibitors of matrix metalloproteinases and are useful in the methods of the instant invention include, but are not limited to, marimastat (described in U.S. Pat. No. 5,700,838), prinomastat (also known as AG3340 and described in U.S. Pat. No. 5,753653), COL-3 (described in U.S. Pat. No. 5,837,696), neovastat (Aeterna) and BMS-275291 (Bristol-Myers-Squibb). Compounds which have inhibitory activity for a matrix metalloproteinase can be readily identified by using assays well-known in the art. For example, see the assays described or cited in PCT Pat. Publ. WO 98/34915 in particular on pp. 24-26.

Angiogenesis inhibitors that inhibit the growth of endothelial cells and are useful in the methods of the instant invention include, but are not limited to, the proteins angiostatin (see U.S. Pat. No. 5,792,845) and endostatin (see U.S. Pat. No. 5,854,205), TNP-470 (described in U.S. Pat. No. 5,196,406), squalamine (described in U.S. Pat. No. 5,840,936), Combrestatin A-4 Prodrug (described in U.S. Pat. No. 5,561,122) and thalidomide.

Angiogenesis inhibitors that inhibit endothelial-specific integrin/survival signaling include, but are not limited to, EMD 121974 (Merck KgaA) and Vitaxin. Such angiogenesis inhibitors also include compounds which selectively antagonize, inhibit or counteract binding of a physiological ligand to the αvβ3 integrin, which selectively antagonize, inhibit or counteract binding of a physiological ligand to the αvβ5 integrin, which antagonize, inhibit or counteract binding of a physiological ligand to both the αvβ3 integrin and the αvβ5 integrin, or which antagonize, inhibit or counteract the activity of the particular integrin(s) expressed on capillary endothelial cells. Antagonists of the α1β1, α2β1, α5β1, α6β1 and α6β4 integrins and antagonists of any combination of αvβ3 integrin, αvβ5 integrin, α1β1, α2β1, α5β1, α6β1 and α6β4 integrins may also be useful to inhibit endothelial-specific integrin/survival signaling.

Angiogenesis inhibitors that block the activators of angiogenesis factors such as fibroblast growth factors, vascular endothelial growth factor and vascular permeability factors include, but are not limited to, interferon-alpha, anti-VEGF antibody (Genentech), SU5416 (Sugen), SU6668 (Sugen), anti-KDR antibody (Imclone-IMC-1C11), Angiozyme and PTK787/ZK22584 (Novartis). Angiogenesis inhibitors that block the activators of angiogenesis factors include inhibitors of KDR; however, inhibitors of KDR may also contribute therapeutically by mechanisms of action separate from inhibition of angiogenesis. Use of inhibitors of KDR in the methods of the instant invention also includes the use of such inhibitors for their non-antiangiogenesis therapeutic properties. Inhibitors of KDR useful in the instant invention include the following compounds:

(a) a compound represented by formula (I) and described in PCT Publ. No. WO 98/54093:

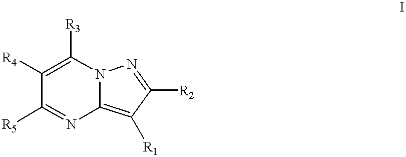

or a pharmaceutically acceptable salt, hydrate or prodrug thereof, wherein

R₁ is H, C-₁₋₁₀ alkyl, C₃₋₆ cycloalkyl, aryl, halo, OH, C₃₋₁₀ heterocyclyl, or C₅₋₁₀ heteroaryl; said alkyl, aryl, heteroaryl and heterocyclyl being optionally substituted with from one to three members selected from R^{a};

R₂ and R₃ are independently H, C₁₋₆ alkyl, aryl, C₃₋₆ cycloalkyl, OH, NO₂, —NH₂, or halogen;

R₄ is H, C₁₋₁₀ alkyl, C₃₋₆ cycloalkyl, C₁₋₆ alkoxy C₂₋₁₀ alkenyl, C₂₋₁₀ alkynyl, aryl, C₃₋₁₀ heterocyclyl, C₁₋₆ alkoxyNR₇R₈, NO₂, OH, —NH₂ or C₅₋₁₀ heteroaryl, said alkyl, alkenyl, alkynyl, aryl, heteroaryl and heterocyclyl being optionally substituted with from one to three members selected from R^{a};

R₅ is H, or C₁₋₆ alkyl, OR, halo, NH₂ or NO₂;

R^{a} is H, C₁₋₁₀ alkyl, halogen, NO₂, OR, —NR, NR₇R₈, R₇R₈, aryl, C₅₋₁₀ heteroaryl or C₃₋₁₀ heterocyclyl,

R is H, or C₁₋₆ alkyl; and

R₇ and R₈ are independently H, C₁₋₁₀ alkyl, C₃₋₆ cycloalkyl, COR, COOR, COO—, aryl, C₃₋₁₀ heterocyclyl, or C₅₋₁₀ heteroaryl or NR₇R₈ can be taken together to form a heterocyclic 5-10 membered saturated or unsaturated ring containing, in addition to the nitrogen atom, one to two additional heteroatoms selected from the group consisting of N, O and S;

(b) a compound represented by formula (II):

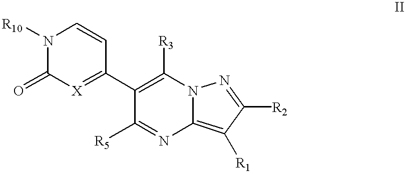

or a pharmaceutically acceptable salt, hydrate or prodrug thereof, wherein:

X is CH or N;

R₁ and R₃ are independently H, C₁₋₁₀ alkyl, C₃₋₆ cycloalkyl, aryl, halo, OH, C₃₋₁₀ heterocyclyl, or C₅₋₁₀ heteroaryl; said alkyl, aryl, heteroaryl and heterocyclyl being optionally substituted with from one to three members selected from R^{a};

R₂ is H, C₁₋₆ alkyl, aryl, C₃₋₆ cycloalkyl, OH, NO₂, —NH₂, or halogen;

R₁₀ is H, or C₁₋₆ alkyl, C₁₋₆ alkylR₉, NHC₁₋₆ alkylR₉, NR₇R₈, O—C₁₋₆ alkylR₉ aryl, C₃₋₁₀ heterocyclyl, said alkyl, aryl and heterocyclyl being optionally substituted with from one to three members selected from R^{a};

R₅ is H, C₁₋₆ alkyl, OH, O—C₁₋₆ alkyl, halo, NH₂ or NO₂;

R^{a} is H, C₁₋₁₀ alkyl, halogen, NO₂, OR, NR₇R₈, CN, aryl, C₅₋₁₀ heteroaryl or C₃₋₁₀ heterocyclyl,

R is H, or C₁₋₆ alkyl;

R₉is aryl, C₃₋₁₀ heterocyclyl, or C₅₋₁₀ heteroaryl said aryl, heteroaryl and heterocyclyl being optionally substituted with from one to three members selected from R^{a}; and

R₇ and R₈ are independently H, C₁₋₁₀ alkyl, C₃₋₆ cycloalkyl, COR, COOR, COO—, aryl, C₃₋₁₀ heterocyclyl, or C₅₋₁₀ heteroaryl or NR₇R₈ can be taken together to form a heterocyclic 5-10 membered saturated or unsaturated ring containing, in addition to the nitrogen atom, one to two additional heteroatoms selected from the group consisting of N, O and S;

(c) a compound represented by formula (III):

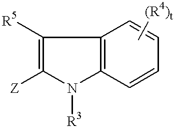

or a pharmaceutically acceptable salt, hydrate or prodrug thereof, wherein

Z is

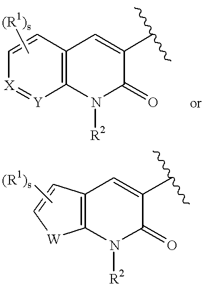

W is S or O;

a is 0 or 1;

b is 0 or 1;

s is 1 or 2;

t is 1, 2, or 3;

X═Y is C═N, N═C, or C═C;

R¹, R⁴ and R⁵ are independently selected from:

1) H,

2) (C═O)ₐO_{b}C₁-C₁₀ alkyl, optionally substituted with one to three substituents selected from R⁶,

3) (C═O)ₐO_{b}aryl, optionally substituted with one to three substituents selected from R⁶,

4) C₂-C₁₀ alkenyl, optionally substituted with one to three substituents selected from R⁶,

5) C₂-C₁₀ alkynyl, optionally substituted with one to three substituents selected from R⁶,

6) CO₂H,

7) halo,

8) OH,

9) O_{b}C₁-C₆ perfluoroalkyl, and

10) (C═O)ₐNR⁷R⁸;

R² and R³ are independently selected from the group consisting of:

1) H,

2) (C═O)OₐC₁-C₆ alkyl,

3) (C═O)Oₐaryl,

4) C₁-C₆ alkyl, and

5) aryl;

R⁶ is:

1) H,

2) (C═O)ₐO_{b}C₁-C₆ alkyl,

3) (C═O)ₐO_{b}aryl,

4) C₂-C₁₀ alkenyl,

5) C₂-C₁₀ alkynyl,

6) heterocyclyl,

7) CO₂H,

8) halo,

9) CN,

10) OH,

11) O_{b}C₁-C₆ perfluoroalkyl, or

12) NR⁷R⁸;

R^{6a} is:

1) H,

2) (C═O)ₐO_{b}C₁-C₆ alkyl,

3) (C═O)ₐO_{b}aryl,

4) C₂-C₁₀ alkenyl,

5) C₂-C₁₀ alkynyl,

6) heterocyclyl,

7) CO₂H,

8) halo,

9) CN,

10) OH,

11) O_{b}C₁-C₆ perfluoroalkyl, or

12) N(C₁-C₆ alkyl)₂;

R⁷ and R⁸ are independently selected from:

1) H,

2) (C═O)O_{b}C₁-C₁₀ alkyl, optionally substituted with one to three substituents selected from R^{6a},

3) (C═O)O_{b}aryl, optionally substituted with one to three substituents selected from R^{6a},

4) C₁-C₁₀ alkyl, optionally substituted with one to three substituents selected from R^{6a},

5) aryl, optionally substituted with one to three substituents selected from R^{6a},

6) C₂-C₁₀ alkenyl, optionally substituted with one to three substituents selected from R^{6a},

7) C₂-C₁₀ alkynyl, optionally substituted with one to three substituents selected from R^{6a}, and

8) heterocyclyl, or

R⁷ and R⁸ can be taken together with the nitrogen to which they are attached to form a 5-7 membered heterocycle containing, in addition to the nitrogen, one or two additional heteroatoms selected from N, O and S, said heterocycle optionally substituted with one to three substituents selected from R^{6a}.

(d) a compound represented by formula (IV):

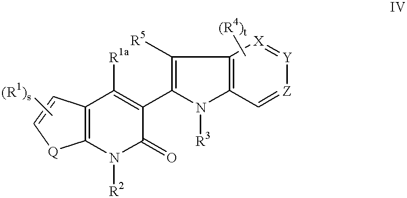

or a pharmaceutically acceptable salt or stereoisomer thereof, wherein

Q is S, O, or —E═D;

X, Y and Z are C or N, so long as only one of X, Y and Z is N;

a is 0 or 1;

b is 0 or 1;

s is 1 or 2;

t is 1, 2, or 3;

m is 0, 1, or 2;

E═D is C═N, N═C, or C═C;

R¹, R^{1a}, R⁴ and R⁵ are independently selected from:

1) H,

2) (C═O)ₐO_{b}C₁-C₁₀ alkyl, optionally substituted with one to three substituents selected from R⁶,

3) (C═O)ₐO_{b}aryl, optionally substituted with one to three substituents selected from R⁶,

4) (C═O)ₐO_{b}C₂-C₁₀ alkenyl, optionally substituted with one to three substituents selected from R⁶,

5) (C═O)ₐO_{b}C₂-C₁₀ alkynyl, optionally substituted with one to three substituents selected from R⁶,

6) SOₘC₁-C₁₀ alkyl, optionally substituted with one to three substituents selected from R⁶,

7) SOₘaryl, optionally substituted with one to three substituents selected from R⁶,

8) CO₂H,

9) halo,

10) CN,

11) OH,

12) O_{b}C₁-C₆ perfluoroalkyl, and

13) (C═O)ₐNR⁷R⁸;

R² and R³ are independently selected from the group consisting of:

1) H,

2) (C═O)OₐC₁-C₁₀ alkyl,

3) (C═O)Oₐaryl,

4) C₁-C₁₀ alkyl,

5) SOₘC₁-C₁₀ alkyl,

6) SOₘaryl,

7) (C═O)ₐO_{b}C₂-C₁₀ alkenyl,

8) (C═O)ₐO_{b}C₂-C₁₀ alkynyl, and

9) aryl,

said alkyl, aryl, alkenyl and alkynyl is optionally substituted with one to three substituents selected from R⁶;

R⁶ is:

1) H,

2) (C═O)ₐO_{b}C₁-C₆ alkyl,

3) (C═O)ₐO_{b}aryl,

4) C₂-C₁₀ alkenyl,

5) C₂-C₁₀ alkynyl,

6) heterocyclyl,

7) CO₂H,

8) halo,

9) CN,

10) OH,

11) oxo,

12) O_{b}C₁-C₆ perfluoroalkyl, or

13) NR⁷R⁸;

R^{6a} is:

1) H,

2) (C═O)ₐO_{b}C₁-C₆ alkyl,

3) (C═O)ₐO_{b}aryl,

4) C₂-C₁₀ alkenyl,

5) C₂-C₁₀ alkynyl,

6) heterocyclyl,

7) CO₂H,

8) halo,

9) CN,

10) OH,

11) oxo,

12) O_{b}C₁-C₆ perfluoroalkyl, or

13) N(C₁-C₆ alkyl)₂;

R⁷ and R⁸ are independently selected from:

1) H,

+P3

2) (C═O)O_{b}C₁-C₁₀ alkyl, optionally substituted with one to three substituents selected from R^{6a},

3) (C═O)O_{b}aryl, optionally substituted with one to three substituents selected from R^{6a},

4) C₁-C₁₀ alkyl, optionally substituted with one to three substituents selected from R^{6a},

5) aryl, optionally substituted with one to three substituents selected from R⁶a,

6) C₂-C₁₀ alkenyl, optionally substituted with one to three substituents selected from R^{6a},

7) C₂-C₁₀ alkynyl, optionally substituted with one to three substituents selected from R^{6a}, and

8) heterocyclyl, or

R⁷ and R⁸ can be taken together with the nitrogen to which they are attached to form a 5-7 membered heterocycle containing, in addition to the nitrogen, one or two additional heteroatoms selected from N, O and S, said heterocycle optionally substituted with one to three substituents selected from R^{6a}.

Examples of compounds which inhibit angiogenesis and are inhibitors or KDR include the following:

4-(3-phenyl-pyrazolo[1,5-a]pyrimidin-6-yl)-1-(3-piperidin-1-yl-propyl)-1H-pyridin-2-one,

1-(2-morpholin-4-yl-ethyl)-4-(3-phenyl-pyrazolo[1,5-a]pyrimidin-6-yl)-1H-pyridin-2-one,

1-(3-dimethylamino-propyl)-4-(3-phenyl-pyrazolo[1,5-a]pyrimidin-6-yl)-1H-pyridin-2-one,

1-(1-methyl-piperidin-3-ylmethyl)-4-(3-phenyl-pyrazolo[1,5-a]pyrimidin-6-yl)-1H-pyridin-2-one,

1-[3-(4-methylpiperazin-1-yl)-propyl)]-4-(3-phenyl-pyrazolo[1,5-a]pyrimidin-6-yl)-1H-pyridin-2-one,

1-(2-dimethylamino-propyl)-4-(3-phenyl-pyrazolo[1,5-a]pyrimidin-6-yl)-1H-pyridin-2-one,

1-(1-dimethylamino-2-methyl-propyl)-4-(3-phenyl-pyrazolo[1,5-a]pyrimidin-6-yl)-1H-pyridin-2-one,

1-[2-(4-cyano-piperidin-1-yl-ethyl]-4-(3-phenyl-pyrazolo[1,5-a]pyrimidin-6-yl)-1H-pyridin-2-one,

1-(3-piperidin-1-yl-propyl)-4-(3-thiophen-3-yl-pyrazolo[1,5-a]pyrimidin-6-yl)-1H-pyridin-2-one,

1-(3-piperidin-1-yl-ethyl)-4-(3-thiophen-3-yl-pyrazolo[1,5-a]pyrimidin-6-yl)-1H-pyridin-2-one,

1-(2-morpholin-4-yl-ethyl)-4-(3-thiophen-3-yl-pyrazolo[1,5-a]pyrimidin-6-yl)-1H-pyridin-2-one,

1-(3-dimethylamino-propyl)-4-(3-thiophen-3-yl-pyrazolo[1,5-a]pyrimidin-6-yl)-1H-pyridin-2-one,

1-(1-methyl-piperidin-3-ylmethyl)-4-(3-thiophen-3-yl-pyrazolo[1,5-a]pyrimidin-6-yl)-1H-pyridin-2-one,

1-[3-(4-methylpiperazin-1-yl)-propyl)]-4-(3-thiophen-3-yl-pyrazolo[1,5-a]pyrimidin-6-yl)-1H-pyridin-2-one,

1-(2-dimethylamino-propyl)-4-(3-thiophen-3-yl-pyrazolo[1,5-a]pyrimidin-6-yl)-1H-pyridin-2-one,

1-(1-dimethylamino-2-methyl-propyl)-4-(3-thiophen-3-yl -pyrazolo[1,5-a]pyrimidin-6-yl)-1H-pyridin-2-one,

1-(3-dimethylamino-propyl)-4-(3-thiophen-3-yl-pyrazolo[1,5-a]pyrimidin-6-yl)-1H-pyridin-2-one,

1-[2-(4-cyano-piperidin-1-yl-ethyl]-4-(3-thiophen-3-yl-pyrazolo [1,5-a]pyrimidin-6-yl)-1H-pyridin-2-one,

4-(3-phenyl-pyrazolo[1,5-a]pyrimidin-6-yl)-1-(3-piperidin-1-yl-propyl)-1H-pyrimidin-2-one,

1-(2-morpholin-4-yl-ethyl)-4-(3-phenyl-pyrazolo[1,5-a]pyrimidin-6-yl)-1H-pyrimidin-2-one,

1-(3-dimethylamino-propyl)-4-(3-phenyl-pyrazolo[1,5-a]pyrimidin-6-yl)-1H-pyrimidin-2-one,

1-(1-methyl-piperidin-3-ylmethyl)-4-(3-phenyl-pyrazolo[1,5-a]pyrimidin-6-yl)-1H-pyrimidin-2-one,

1 1-[3-(4-methylpiperazin-1-yl)-propyl)]-4-(3-phenyl-pyrazolo[1,5-a]pyrimidin-6-yl)-1H-pyrimidin-2-one,

1-(2-dimethylamino-propyl)-4-(3-phenyl-pyrazolo[1,5-a]pyrimidin-6-yl)-1H-pyrimidin-2-one,

1-(1-dimethylamino-2-methyl-propyl)-4-(3-phenyl-pyrazolo[1,5-a]pyrimidin-6-yl)-1H-pyrimidin-2-one,

1-[2-(4-cyano-piperidin-1-yl-ethyl]-4-(3-phenyl-pyrazolo[1,5-a]pyrimidin-6-yl)-pyrimidin-2-one

3-[5-(2-piperidin-1-yl-ethoxy)-1H-indol-2-yl]-1H-quinolin-2-one,

3-[5-(2-pyrrolidin-1-yl-ethoxy)-1H-indol-2-yl]-1H-quinolin-2-one,

3-[5-(2-morpholin-4-yl-ethoxy)-1H-indol-2-yl]-1H-quinolin-2-one,

3-[5-(3-dimethylamino-2-methyl-propoxy)-1H-indol-2-yl]-1H-quinolin-2-one,

3-[5-(3-piperidin-1-yl-propoxy)-1H-indol-2-yl]-1H-quinolin-2-one,

3-(5-{2-[benzyl-(2-methoxy-ethyl)-amino]-ethoxy}-1H-indol-2-yl)-1H-quinolin-2-one,

3-[5-(2-diethylamino-ethoxy)-1H-indol-2-yl]-1H-quinolin-2-one,

3-{5-[3-(benzyl-methyl-amino)-propoxy]-1H-indol-2-yl}-1H-quinolin-2-one,

1-{2-[2-(2-oxo-1,2-dihydro-quinolin-3-yl)-1H-indol-5-yloxy]-ethyl}-piperidine-4-carbonitrile,

3-{5-[3-(4-methyl-piperazin-1-yl)-propoxy]-1H-indol-2-yl}-1H-quinolin-2-one,

3-[5-(3-morpholin-4-yl-propoxy)-1H-indol-2-yl]-1H-quinolin-2-one,

3-(5-{2-[bis-(2-methoxy-ethyl)-amino]-ethoxy}-1H-indol-2-yl)-1H-quinolin-2-one,

3-(5-{2-[ethyl-(2-methoxy-ethyl)-amino]-ethoxy}-1H-indol-2-yl)-1H-quinolin-2-one,

3-(5-{2-[(2-methoxy-ethyl)-methyl-amino]-ethoxy}-1H-indol-2-yl)-1H-quinolin-2-one,

3-(1H-indol-2-yl)-1H-quinolin-2-one

3-(5-methoxy-1H-pyrrolo[3,2-b]pyridin-2-yl)-1H-quinolin-2-one;

3-(1H-pyrrolo[2,3-c]pyridin-2-yl)-1H-quinolin-2-one;

3-(1H-pyrrolo[3,2-c]pyridin-2-yl)-1H-quinolin-2-one;

3-(1H-pyrrolo[3,2-b]pyridin-2-yl)-1H-quinolin-2-one;

3-(5-methoxy-1H-pyrrolo[2,3-c]pyridin-2-yl)-1H-quinolin-2-one;

3-(5-oxo-4,5-dihydro-1H-pyrrolo[3,2-b]pyridin-2-yl)-1H-quinolin-2-one;

3-(5-oxo-5,6-dihydro-1H-pyrrolo[2,3-c]pyridin-2-yl)-1H-quinolin-2-one;

3-(4-oxo-4,5-dihydro-1H-pyrrolo[3,2-c]pyridin-2-yl)-1H-quinolin-2-one,

3-(4-fluorophenyl)-6-(4-pyridyl) pyrazolo(1,5-A)pyrimidine,

3-(3-chlorophenyl)-6-(4-pyridyl) pyrazolo(1,5-A)pyrimidine,

3-(3,4-methylenedioxypheny)-6-(4-pyridyl) pyrazolo(1,5-A)pyrimidine,

3-(phenyl)-6-(4-pyrimidyl)pyrazolo(1,5-A)pyrimidine,

3-(4-fluorophenyl)-6-(4-pyrimidyl)pyrazolo(1,5-A)pyrimidine,

3-(3-chlorophenyl)-6-(4-pyrimidyl)pyrazolo(1,5-A)pyrimidine,

3-(3-thienyl)-6-(4-pyrimidyl)pyrazolo(1,5-A)pyrimidine,

3-(3-acetamidophenyl)-6-(4-methylphenyl)pyrazolo(1,5-A)pyrimidine,

3-(3-thienyl)-6-(4-methylphenyl)pyrazolo(1,5-A)pyrimidine,

3-(phenyl)-6-(4-methoxyphenyl)pyrazolo(1,5-A)pyrimidine,

3-(3-acetamidophenyl)-6-(4-methoxyphenyl)pyrazolo(1,5-A)pyrimidine,

3-(3-thienyl)-6-(4-methoxyphenyl)pyrazolo(1,5-A)pyrimidine,

3-(phenyl)-6-(4-methoxyphenyl)pyrazolo(1,5-A)pyrimidine,

3-(4-pyridyl)-6-(4-methoxyphenyl)pyrazolo(1,5-A)pyrimidine,

3-(phenyl)-6-(4-chlorophenyl)pyrazolo(1,5-A)pyrimidine.

3-(4-pyridyl)-6-(4-chlorophenyl)pyrazolo(1,5-A)pyrimidine,

3-(phenyl)-6-(4-methylphenyl)pyrazolo(1,5-A)pyrimidine,

3-(4-pyridyl)-6-(4-methylphenyl)pyrazolo(1,5-A)pyrimidine,

3-(phenyl)-6-(2-pyridyl)pyrazolo(1,5-A)pyrimidine,

3-(4-pyridyl)-6-(2-pyridyl)pyrazolo(1,5-A)pyrimidine,

3-(phenyl)-6-(4-pyrimidyl)pyrazolo(1,5-A)pyrimidine,

3-(4-pyridyl)-6-(4-pyrimidyl)pyrazolo(1,5-A)pyrimidine,

3-(phenyl)-6-(2-pyrazinyl)pyrazolo(1,5-A)pyrimidine,

3-(4-pyridyl)-6-(2-pyrazinyl)pyrazolo(1,5-A)pyrimidine,

3-(3-pyridyl)-6-(4-methoxyphenyl)pyrazolo(1,5-A)pyrimidine,

3-(phenyl)-6-(4-pyridyl)pyrazolo(1,5-A)pyrimidine,

3-(3-pyridyl)-6-(4-pyridyl)pyrazolo(1,5-A)pyrimidine,

3-(4 pyridyl)-6-(4-methoxyphenyl)pyrazolo(1,5-A)pyrimidine,

3-(3-thienyl)-6-(4-methoxyphenyl)pyrazolo(1,5-A)pyrimidine,

3-(3-thienyl)-6-(4-hydroxyphenyl)pyrazolo(1,5-A)pyrimidine,

3-(3-thienyl)-6-(4-(2-(4-morpholinyl)ethoxy)phenyl)pyrazolo(1,5-A)pyrimidine,

3-(3-thienyl)-6-(cyclohexyl)pyrazolo(1,5-A)pyrimidine,

3-(bromo)-6-(4-methoxyphenyl)pyrazolo(1,5-A)pyrimidine,

3-(bromo)-6-(4-pyrimidyl)pyrazolo(1,5-A)pyrimidine,

3-(phenyl)-6-(2-(3-carboxy)pyridyl)pyrazolo(1,5-A)pyrimidine,

3-(3-thienyl)-6-(4-pyridyl)pyrazolo(1,5-A)pyrimidine.

or a pharmaceutically acceptable salt or optical isomer thereof.

Compounds which are inhibitors of angiogenesis and are inhibitors of KDR and are therefore useful in the present invention, and methods of synthesis thereof, can be found in the following patents, pending applications and publications, which are herein incorporated by reference:

WO 98/54093 (Dec. 3, 1998); U.S. Ser. No. 09/086,152 filed on May 28, 1998; U.S. Ser. No. 09/424,132 filed on Nov. 14, 1999;

WO 99/16755 (Apr. 8, 1999); U.S. Ser. No. 09/143,881 filed on Aug. 31, 1998; WO 00/12089 (Mar. 9, 2000); U.S. Ser. No. 09/266,331, filed on Mar. 11, 1999;

WO 00/02871 (Jan. 20, 2000); U.S. Ser. No. 09/343,652 filed on Jun. 29, 1999;

U.S. Ser. No. 09/480,717 filed on Jan. 7, 2000;

U.S. Ser. No. 09/519,780 filed on Mar. 7, 2000;

U.S. Ser. No. 60/153,348 filed on Sep. 10, 1999;

U.S. Ser. No. 60/160,362 filed on Oct. 19, 1999;

U.S. Ser. No. 60/160,356 filed on Oct. 19, 1999;

U.S. Ser. No. 60/185,023 filed on Feb. 25, 2000;

U.S. Ser. No. 60/185,024 filed on Feb. 25, 2000;

PSA conjugates that are useful in the methods of the instant invention and are identified by the properties described hereinabove include:

a) a compound represented by the formula IX:

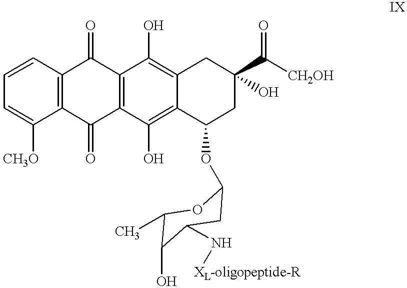

wherein:

oligopeptide is an oligopeptide which is selectively recognized by the free prostate specific antigen (PSA) and is capable of being proteolytically cleaved by the enzymatic activity of the free prostate specific antigen;

X_{L} is absent or is an amino acid selected from:

a) phenylalanine,

b) leucine,

c) valine,

d) isoleucine,

e) (2-naphthyl)alanine,

f) cyclohexylalanine,

g) diphenylalanine,

h) norvaline, and

j) norleucine;

R is hydrogen or —(C═O)R¹; and

R¹ is C₁-C₆-alkyl or aryl,

or the pharmaceutically acceptable salt thereof;

b) a compound represented by the formula X:

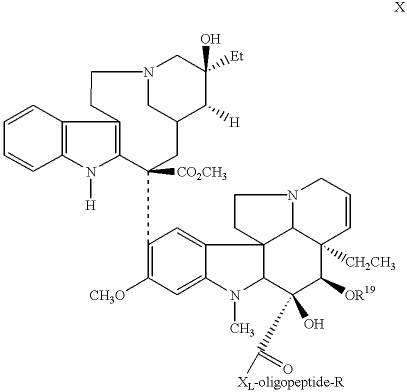

wherein:

oligopeptide is an oligopeptide which is selectively recognized by the free prostate specific antigen (PSA) and is capable of being proteolytically cleaved by the enzymatic activity of the free prostate specific antigen;

X_{L} is absent or is an amino acid selected from:

a) phenylalanine,

b) leucine,

c) valine,

d) isoleucine,

e) (2-naphthyl)alanine,

f) cyclohexylalanine,

g) diphenylalanine,

h) norvaline, and

j) norleucine; or

X_{L} is —NH—(CH₂)ₙ—NH—

R is hydrogen or —(C═O)R¹;

R¹ is C₁-C₆-alkyl or aryl;

R¹⁹ is hydrogen or acetyl; and

n is 1, 2, 3, 4 or 5,

or the pharmaceutically acceptable salt thereof;

c) a compound represented by the formula XI:

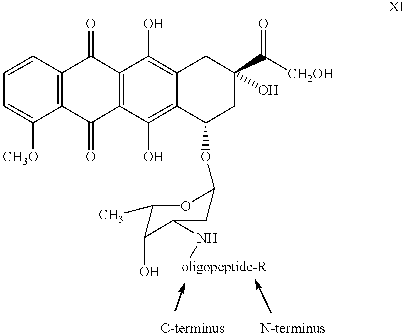

wherein:

oligopeptide is an oligopeptide which is selectively recognized by the free prostate specific antigen (PSA) and is capable of being proteolytically cleaved by the enzymatic activity of the free prostate specific antigen, wherein the oligopeptide comprises a cyclic amino acid of the formula:

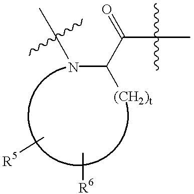

and wherein the C-terminus carbonyl is covalently bound to the amine of doxorubicin;

R is selected from

a) hydrogen,

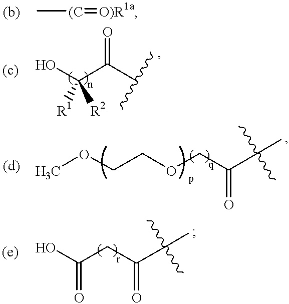

R¹ and R² are independently selected from: hydrogen, OH, C₁-C₆ alkyl, C₁-C₆ alkoxy, C₁-C₆ aralkyl and aryl;

R^{1a} is C₁-C₆-alkyl, hydroxylated aryl, polyhydroxylated aryl or aryl;

R⁵ is selected from HO— and C₁-C₆ alkoxy;

R⁶ is selected from hydrogen, halogen, C₁-C₆ alkyl, HO— and C₁-C₆ alkoxy; and

n is 1, 2, 3 or 4;

p is zero or an integer between 1 and 100;

q is 0 or 1, provided that if p is zero, q is 1;

r is an integer between 1 and 10; and

t is 3 or 4;

or a pharmaceutically acceptable salt thereof;

d) a compound represented by the formula XII:

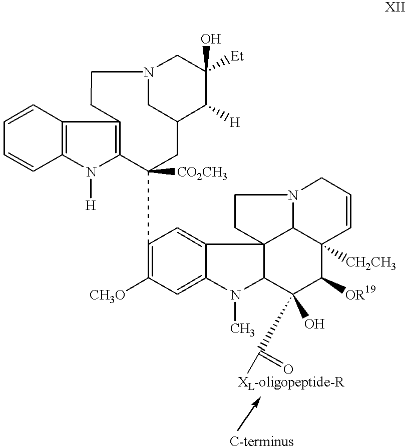

wherein:

oligopeptide is an oligopeptide which is selectively recognized by the free prostate specific antigen (PSA) and is capable of being proteolytic ally cleaved by the enzymatic activity of the free prostate specific antigen, and the oligopeptide comprises a cyclic amino acid of the formula:

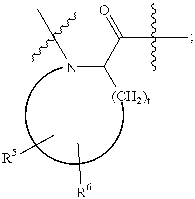

XL is —NH—(CH2)u—NH—

R is selected from

a) hydrogen,

b) —(C═O)R^{1a},

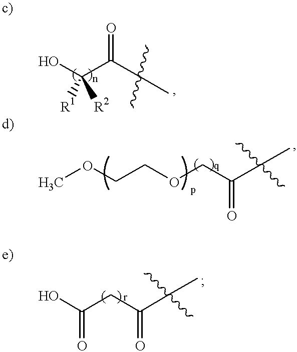

R¹ and R² are independently selected from: hydrogen, OH, C₁-C₆ alkyl, C₁-C₆ alkoxy, C₁-C₆ aralkyl and aryl;

R^{1a} is C₁-C₆-alkyl, hydroxylated aryl, polyhydroxylated aryl or aryl,

R¹⁹ is hydrogen, (C₁-C₃ alkyl)-CO, or chlorosubstituted (C₁-C₃ alkyl)-CO;

n is 1, 2, 3 or 4;

p is zero or an integer between 1 and 100;

q is 0 or 1, provided that if p is zero, q is 1;

r is 1, 2 or 3;

t is 3 or 4;

u is 1, 2, 3, 4 or 5,

or the pharmaceutically acceptable salt thereof;

e) a compound represented by the formula XIII:

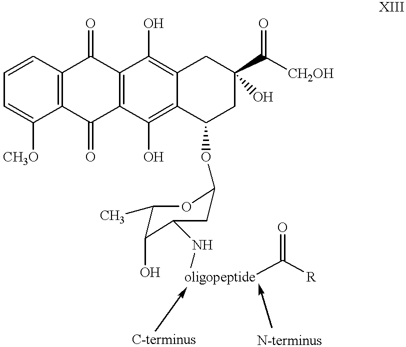

wherein:

oligopeptide is an oligopeptide which is selectively recognized by the free prostate specific antigen (PSA) and is capable of being proteolytically cleaved by the enzymatic activity of the free prostate specific antigen, and wherein the C-terminus carbonyl is covalently bound to the amine of doxorubicin and the N-terminus amine is covalently bound to the carbonyl of the blocking group;

R is selected from

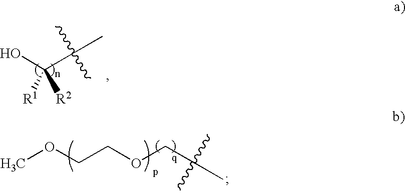

R¹ and R² are independently selected from: hydrogen, OH, C₁-C₆ alkyl, C₁-C₆ alkoxy, C₁-C₆ aralkyl and aryl;

n is 1, 2, 3 or 4;

p is zero or an integer between 1 and 100;

q is 0 or 1, provided that if p is zero, q is 1;

or the pharmaceutically acceptable salt thereof;

f) a compound represented by the formula XIV:

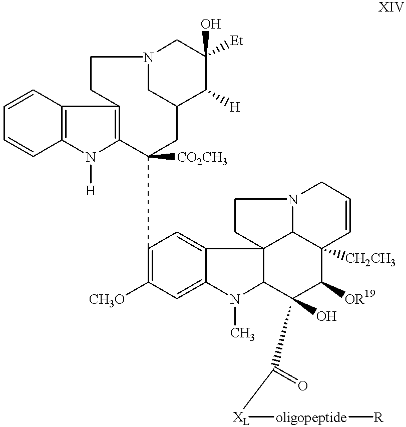

wherein:

oligopeptide is an oligopeptide which is selectively recognized by the free prostate specific antigen (PSA) and is capable of being proteolytically cleaved by the enzymatic activity of the free prostate specific antigen;

X_{L} is —NH—(CH₂)ᵣ—NH—

R is selected from

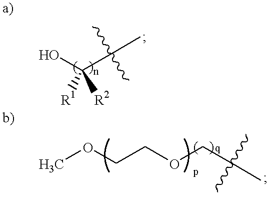

R¹ and R² are independently selected from: hydrogen, OH, C₁-C₆ alkyl, C₁-C₆ alkoxy, C₁-C₆ aralkyl and aryl;

R¹⁹ is hydrogen, (C₁-C₃ alkyl)-CO, or chlorosubstituted (C₁-C₃ alkyl)-CO;

n is 1, 2, 3 or 4;

p is zero or an integer between 1 and 100;

q is 0 or 1, provided that if p is zero, q is 1;

r is 1, 2, 3, 4 or 5,

or the pharmaceutically acceptable salt thereof;

g) a compound represented by the formula XV:

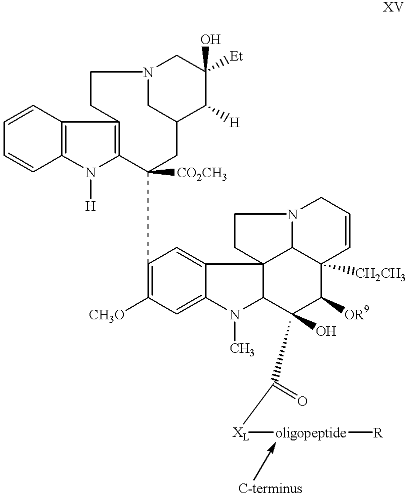

wherein:

oligopeptide is an oligopeptide which is selectively recognized by the free prostate specific antigen (PSA) and is capable of being proteolytically cleaved by the enzymatic activity of the free prostate specific antigen,

X_{L} is —NH—(CH₂)ᵤ—W—(CH₂)ᵤ—NH—

R is selected from

a) hydrogen,

b) —(C═O)R^{1a},

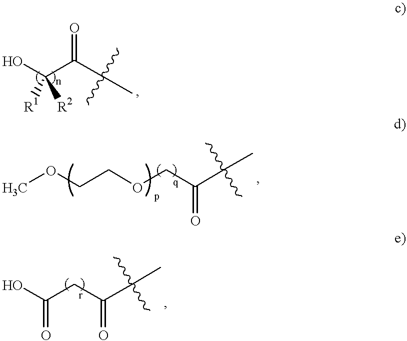

f) ethoxysquarate, and

g) cotininyl;

R¹ and R² are independently selected from: hydrogen, OH, C₁-C₆ alkyl, C₁-C₆ alkoxy, C₁-C₆ aralkyl and aryl;

R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl;

R⁹ is hydrogen, (C₁-C₃ alkyl)-CO, or chlorosubstituted (C₁-C₃ alkyl)-CO;

W is selected from cyclopentyl, cyclohexyl, cycloheptyl or bicyclo[2,2,2]octanyl;

n is 1, 2, 3 or 4;

p is zero or an integer between 1 and 100;

q is 0 or 1, provided that if p is zero, q is 1;

r is 1, 2 or 3;

t is 3 or 4;

u is 0, 1, 2 or 3,

or the pharmaceutically acceptable salt thereof; and

h) a compound represented by the formula XVI:

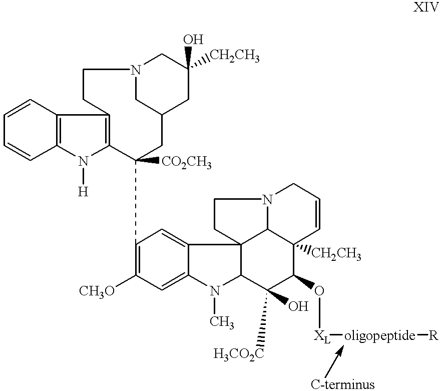

wherein:

oligopeptide is an oligopeptide which is selectively recognized by the free prostate specific antigen (PSA) and is capable of being proteolytically cleaved by the enzymatic activity of the free prostate specific antigen,

X_{L} is selected from: a bond, —C(O)—(CH₂)ᵤ—W—(CH₂)ᵤ—O— and —C(O)—(CH₂)ᵤ—W—(CH₂)ᵤ—NH—;

R is selected from

a) hydrogen,

b) —(C═O)R^{1a},

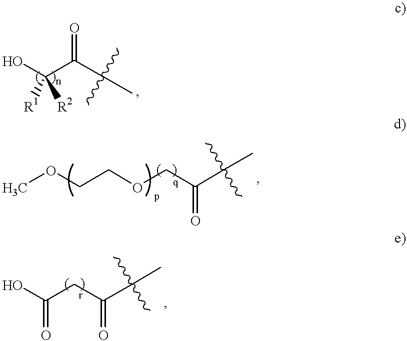

f) ethoxysquarate, and

g) cotininyl;

R¹ and R² are independently selected from: hydrogen, OH, C₁-C₆ alkyl, C₁-C₆ alkoxy, C₁-C₆ aralkyl and aryl;

R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl;

R⁹ is hydrogen, (C₁-C₃ alkyl)-CO, or chlorosubstituted (C₁-C₃ alkyl)-CO;

W is selected from a branched or straight chain C₁-C₆-alkyl, cyclopentyl, cyclohexyl, cycloheptyl or bicyclo[2.2.2]octanyl;

n is 1, 2, 3 or 4;

p is zero or an integer between 1 and 100;

q is 0 or 1, provided that if p is zero, q is 1;

r is 1, 2 or 3;

t is 3 or 4;

u is 0, 1, 2 or 3;

or the pharmaceutically acceptable salt or optical isomer thereof.

Examples of compounds which are PSA conjugates include the following:

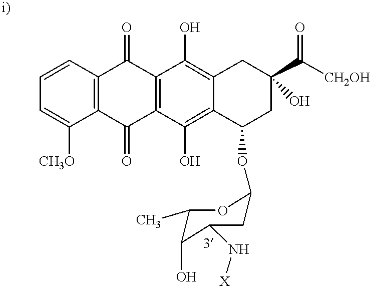

wherein X is:

AsnLysIleSerTyrGlnSer—(SEQ.ID.NO.: 1),

AsnLysIleSerTyrGlnSerSer—(SEQ.ID.NO.: 2),

AsnLysIleSerTyrGlnSerSerSer—(SEQ.ID.NO.:3),

AsnLysIleSerTyrGlnSerSerSerThr—(SEQ.ID.NO.:4),

AsnLysIleSerTyrGlnSerSerSerThrGlu—(SEQ.ID.NO.: 5),

AlaAsnLysIleSerTyrGlnSerSerSerThrGlu—(SEQ.ID.NO.: 6),

Ac—AlaAsnLysIleSerTyrGlnSerSerSerThr—(SEQ.ID.NO.: 7),

Ac—AlaAsnLysIleSerTyrGInSerSerSerThrLeu—(SEQ.ID.NO.: 8),

Ac—AlaAsnLysAlaSerTyrGInSerAlaSerThrLeu—(SEQ.ID.NO.: 9),

Ac—AlaAsnLysAlaSerTyrGlnSerAlaSerLeu—(SEQ.ID.NO.: 10),

Ac—AlaAsnLysAlaSerTyrGlnSerSerSerLeu—(SEQ.ID.NO.: 11),

Ac—AlaAsnLysAlaSerTyrGlnSerSerLeu—(SEQ.ID.NO.: 12),

Ac—SerTyrGlnSerSerSerLeu—(SEQ.ID.NO.: 13),

Ac—hArgTyrGlnSerSerSerLeu—(SEQ.ID.NO.: 14).

Ac—LysTyrGlnSerSerSerLeu—(SEQ.ID.NO.: 15),

Ac—LysTyrGinSerSerNle—(SEQ.ID.NO.: 16),

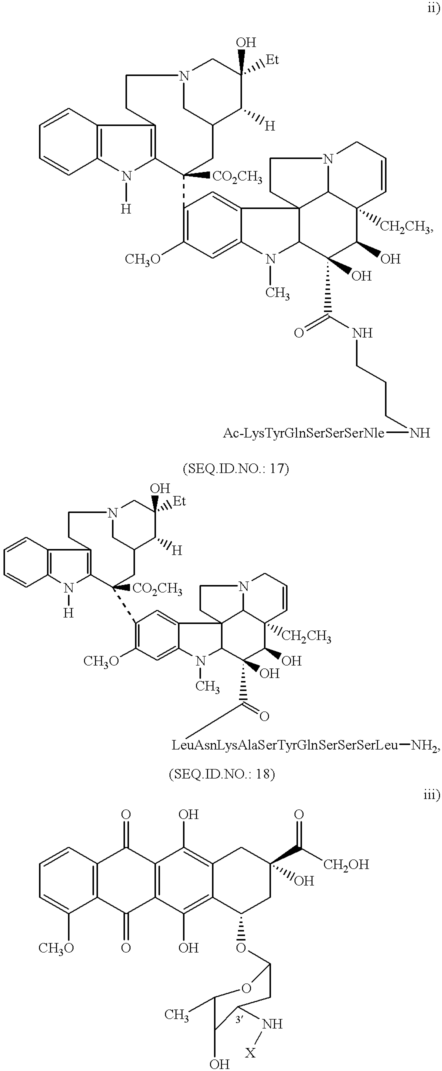

wherein X is:

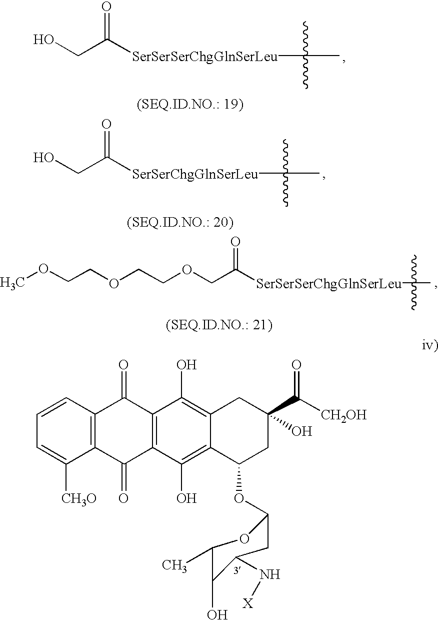

wherein X is:

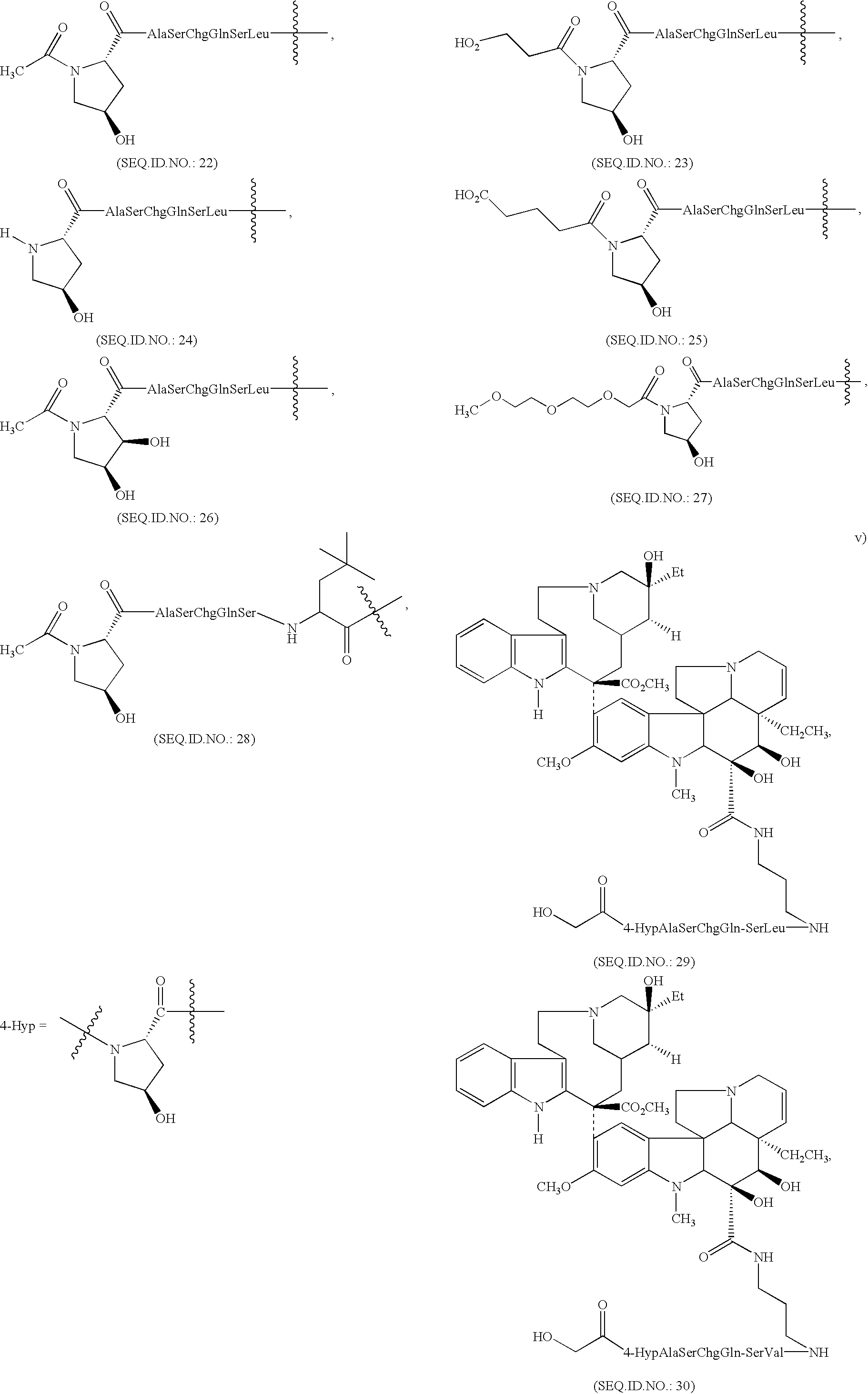

wherein X is

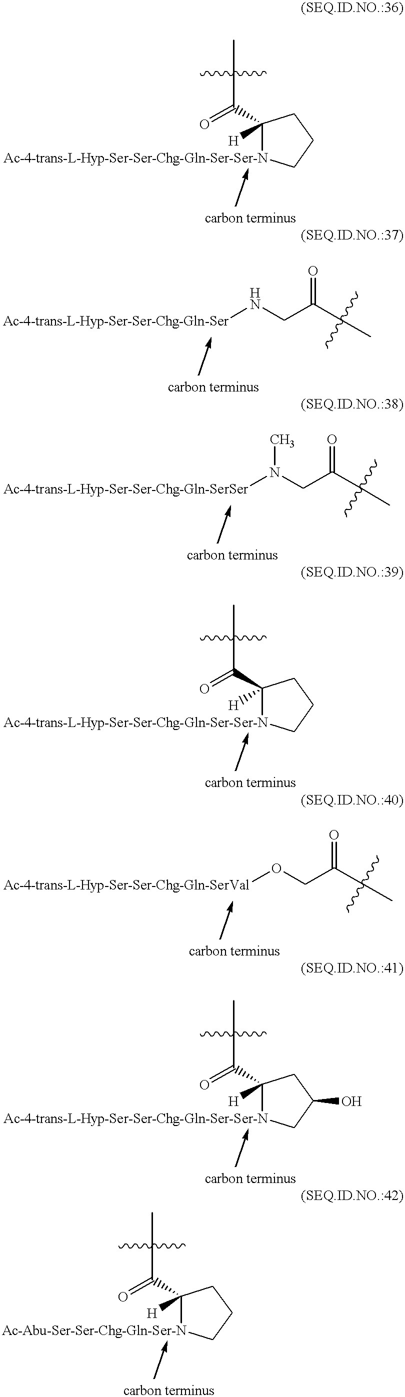

or the pharmaceutically acceptable salt or optical isomer thereof.

Preferably the method of the instant invention comprises the PSA conjugate

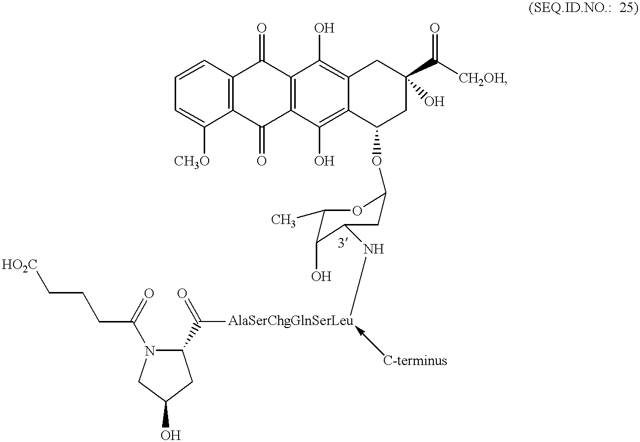

or the pharmaceutically acceptable salt thereof.

Compounds which are PSA conjugates and are therefore useful in the present invention, and methods of synthesis thereof, can be found in the following patents, pending applications and publications, which are herein incorporated by reference:

U.S. Pat. No. 5,599,686 granted on Feb. 4, 1997;

WO 96/00503 (Jan. 11, 1996); U.S. Ser. No. 08/404,833 filed on Mar. 15, 1995; U.S. Ser. No. 08/468,161 filed on Jun. 6, 1995;

U.S. Pat. No. 5,866,679 granted on Feb. 2, 1999;

WO 98/10651 (Mar. 19, 1998); U.S. Ser. No. 08/926,412 filed on Sep. 9, 1997;

U.S. Pat. No. 5,948,750 granted on Sep. 7, 1999, WO 98/18493 (May 7, 1998); U.S. Ser. No. 08/950,805 filed on Oct. 14, 1997;

U.S. Ser. No. 09/112,656 filed on Jul. 9, 1998; U.S. Ser. No. 60/052,195 filed on Jul. 10, 1997; and

U.S. Ser. No. 09/193,365 filed on Nov. 17, 1998; U.S. Ser. No. 60/067,110 filed on Dec. 2, 1997.

U.S. Ser. No. 09/262,538 filed on Mar. 4, 1999; U.S. Ser. No. 60/067,110 filed on March, 1998.

Compounds which are described as prodrugs wherein the active therapeutic agent is release by the action of enzymatically active PSA and therefore may be useful in the present invention, and methods of synthesis thereof, can be found in the following patents, pending applications and publications, which are herein incorporated by reference:

WO 98/52966 (Nov. 26, 1998).

All patents, publications and pending patent applications identified above are hereby incorporated by reference.

With respect to the compounds of formulas I-a through VI and VIIIA the following definitions apply:

The term “alkyl” refers to a monovalent alkane (hydrocarbon) derived radical containing from 1 to 15 carbon atoms unless otherwise defined. It may be straight, branched or cyclic. Preferred straight or branched alkyl groups include methyl, ethyl, propyl, isopropyl, butyl and t-butyl. Preferred cycloalkyl groups include cyclopentyl and cyclohexyl.

When substituted alkyl is present, this refers to a straight, branched or cyclic alkyl group as defined above, substituted with 1-3 groups as defined with respect to each variable.

Heteroalkyl refers to an alkyl group having from 2-15 carbon atoms, and interrupted by from 1-4 heteroatoms selected from O, S and N.

The term “alkenyl” refers to a hydrocarbon radical straight, branched or cyclic containing from 2 to 15 carbon atoms and at least one carbon to carbon double bond. Preferably one carbon to carbon double bond is present, and up to four non-aromatic (non-resonating) carbon-carbon double bonds may be present. Examples of alkenyl groups include vinyl, allyl, isopropenyl, pentenyl, hexenyl, heptenyl, cyclopropenyl, cyclobutenyl, cyclopentenyl, cyclohexenyl, 1-propenyl, 2-butenyl, 2-methyl-2-butenyl, isoprenyl, farnesyl, geranyl, geranylgeranyl and the like. Preferred alkenyl groups include ethenyl, propenyl, butenyl and cyclohexenyl. As described above with respect to alkyl, the straight, branched or cyclic portion of the alkenyl group may contain double bonds and may be substituted when a substituted alkenyl group is provided.

The term “alkynyl” refers to a hydrocarbon radical straight, branched or cyclic, containing from 2 to 15 carbon atoms and at least one carbon to carbon triple bond. Up to three carbon-carbon triple bonds may be present. Preferred alkynyl groups include ethynyl, propynyl and butynyl. As described above with respect to alkyl, the straight, branched or cyclic portion of the alkynyl group may contain triple bonds and may be substituted when a substituted alkynyl group is provided.

Aryl refers to aromatic rings e.g., phenyl, substituted phenyl and like groups as well as rings which are fused, e.g., naphthyl and the like. Aryl thus contains at least one ring having at least 6 atoms, with up to two such rings being present, containing up to 10 atoms therein, with alternating (resonating) double bonds between adjacent carbon atomsExamples of aryl groups include phenyl, naphthyl, anthracenyl, biphenyl, tetrahydronaphthyl, indanyl, phenanthrenyl and the like. The preferred aryl groups are phenyl and naphthyl. Aryl groups may likewise be substituted as defined below. Preferred substituted aryls include phenyl and naphthyl substituted with one or two groups.

The term “heteroaryl” refers to a monocyclic aromatic hydrocarbon group having 5 or 6 ring atoms, or a bicyclic aromatic group having 8 to 10 atoms, containing at least one heteroatom, O, S or N, in which a carbon or nitrogen atom is the point of attachment, and in which one additional carbon atom is optionally replaced by a heteroatom selected from O or S, and in which from 1 to 3 additional carbon atoms are optionally replaced by nitrogen heteroatoms. The heteroaryl group is optionally substituted with up to three groups.

Heteroaryl thus includes aromatic and partially aromatic groups which contain one or more heteroatoms. Examples of this type are thiophene, purine, imidazopyridine, pyridine, oxazole, thiazole, oxazine, pyrazole, tetrazole, imidazole, pyridine, pyrimidine, pyrazine and triazine. Examples of partially aromatic groups are tetrahydro-imidazo[4,5-c]pyridine, phthalidyl and saccharinyl, as defined below.

The term heterocycle or heterocyclic, as used herein, represents a stable 5- to 7-membered monocyclic or stable 8- to 11-membered bicyclic or stable 11-15 membered tricyclic heterocycle ring which is either saturated or unsaturated, and which consists of carbon atoms and from one to four heteroatoms selected from the group consisting of N, O, and S, and including any bicyclic group in which any of the above-defined hetero-cyclic rings is fused to a benzene ring. The heterocyclic ring may be attached at any heteroatom or carbon atom which results in the creation of a stable structure. Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydro-benzothienyl, dihydrobenzothiopyranyl, dihydrobenzothio-pyranyl sulfone, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, 2-oxopiperazinyl, 2-oxopiperidinyl, 2-oxopyrrolidinyl, piperidyl, piperazinyl, pyridyl, pyridyl N-oxide, pyridonyl, pyrazinyl, pyrazolidinyl, pyrazolyl, pyrimidinyl, pyrrolidinyl, pyrrolyl, quinazolinyl, quinolinyl, quinolinyl N-oxide, quinoxalinyl, tetrahydrofuryl, tetrahydroisoquinolinyl, tetrahydro-quinolinyl, thiamorpholinyl, thiamorpholinyl sulfoxide, thiazolyl, thiazolinyl, thienofuryl, thienothienyl, and thienyl. Preferably, heterocycle is selected from imidazolyl, 2-oxopyrrolidinyl, piperidyl, pyridyl and pyrrolidinyl.

The terms “substituted aryl”, “substituted heterocycle” and “substituted cycloalkyl” are intended to include the cyclic group which is substituted with 1 or 2 substitutents selected from the group which includes but is not limited to F, Cl, Br, CF₃, NH₂, N(C₁-C₆ alkyl)₂, NO₂, CN, (C₁-C₆ alkyl)O—, —OH, (C₁-C₆ alkyl)S(O)ₘ—, (C₁-C₆ alkyl)C(O)NH—, H₂N—C(NH)—, (C₁-C₆ alkyl)C(O)—, (C₁-C₆ alkyl)OC(O)—, N₃, (C₁-C₆ alkyl)OC(O)NH— and C₁-C₂₀ alkyl.

The compounds used in the present method may have asymmetric centers and occur as racemates, racemic mixtures, and as individual diastereomers, with all possible isomers, including optical isomers, being included in the present invention. Unless otherwise specified, named amino acids are understood to have the natural “L” stereoconfiguration.

With respect to the compounds of formulas VII through XIV the following definitions apply:

As used herein, “oligopeptide” is preferably a peptide comprising from about 5 amino acids to about 100 amino acids. More preferably, “oligopeptide” is a peptide comprising from about 5 amino acids to about 15 amino acids.

The terms “selective” and “selectively” as used in connection with recognition by PSA and the proteolytic PSA cleavage mean a greater rate of cleavage of an oligopeptide component of the instant invention by free PSA relative to cleavage of an oligopeptide which comprises a random sequence of amino acids. Therefore, the oligopeptide component of the instant invention is a preferred substrate of free PSA. The terms “selective” and “selectively” also indicate that the oligopeptide is proteolytically cleaved by free PSA between two specific amino acids in the oligopeptide.

As used herein, “alkyl” and the alkyl portion of aralkyl and similar terms, is intended to include both branched and straight-chain saturated aliphatic hydrocarbon groups having the specified number of carbon atoms; “alkoxy” represents an alkyl group of indicated number of carbon atoms attached through an oxygen bridge.

As used herein, “cycloalkyl” is intended to include non-aromatic cyclic hydrocarbon groups having the specified number of carbon atoms. Examples of cycloalkyl groups include cyclopropyl, cyclobutyl, cyclopentyl, cyclohexyl and the like.

“Halogen” or “halo” as used herein means fluoro, chloro, bromo and iodo.

As used herein, “aryl,” and the aryl portion of aralkyl and aroyl, is intended to mean any stable monocyclic or bicyclic carbon ring of up to 7 members in each ring, wherein at least one ring is aromatic. Examples of such aryl elements include phenyl, naphthyl, tetrahydro-naphthyl, indanyl, biphenyl, phenanthryl, anthryl or acenaphthyl.

As used herein, the term “hydroxylated” represents substitution on a substitutable carbon of the ring system being so described by a hydroxyl moiety. As used herein, the term “poly-hydroxylated” represents substitution on two or more substitutable carbon of the ring system being so described by 2, 3 or 4 hydroxyl moieties.

As used herein, the term “chlorosubstituted C₁-C₃-alkyl-CO—” represents a acyl moiety having the designated number of carbon atoms attached to a carbonyl moiety wherein one of the carbon atoms is substituted with a chlorine. Example of such chlorosubstituted elements include but are not limited to chloroacetyl, 2-chloropropionyl, 3-chloropropionyl and 2-chlorobutyroyl.

As used herein, the term “PEG” represents certain polyethylene glycol containing substituents having the designated number of ethyleneoxy subunits. Thus the term PEG(2) represents

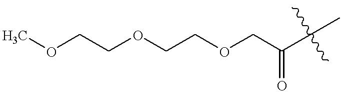

and the term PEG(6) represents

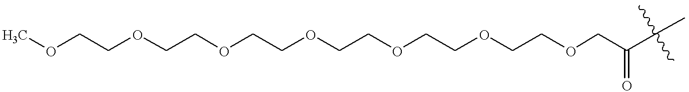

As used herein, the term “(d)(2,3-dihydroxypropionyl)” represents the following structure:

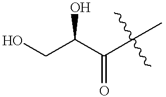

As used herein, the term “(2R, 3S) 2,3,4-trihydroxybutanoyl” represents the following structure:

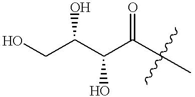

As used herein, the term “quinyl” represents the following structure:

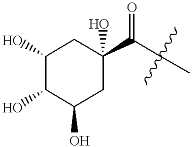

or the diastereomer thereof.

As used herein, the term “cotiminyl” represents the following structure:

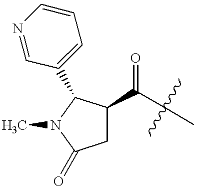

or the diastereomer thereof.

As used herein, the term “gallyl” represents the following structure:

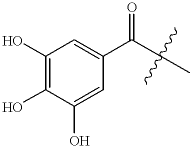

As used herein, the term “4-ethoxysquarate” represents the following structure:

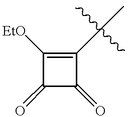

The structure

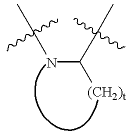

represents a cyclic amine moiety having 5 or 6 members in the ring, such a cyclic amine which may be optionally fused to a phenyl or cyclohexyl ring. Examples of such a cyclic amine moiety include, but are not limited to, the following specific structures:

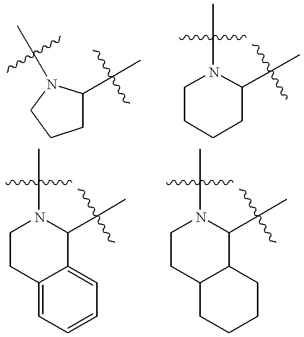

The pharmaceutically acceptable salts of the PSA conjugate compounds of this invention include the conventional non-toxic salts of the compounds of this invention as formed, e.g., from non-toxic inorganic or organic acids. For example, such conventional non-toxic salts include those derived from inorganic acids such as hydrochloric, hydrobromic, sulfuric, sulfamic, phosphoric, nitric and the like: and the salts prepared from organic acids such as acetic, propionic, succinic, glycolic, stearic, lactic, malic, tartaric, citric, ascorbic, pamoic, maleic, hydroxymaleic, phenyl-acetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

The term “pharmaceutically acceptable salts” also refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, such as arginine, betaine, caffeine, choline, N,N⁻dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like, and basic ion exchange resins.

The pharmaceutically acceptable salts of the present invention can be synthesized by conventional chemical methods. Generally, the salts are prepared by reacting the free base or acid with stoichiometric amounts or with an excess of the desired salt-forming inorganic or organic acid or base, in a suitable solvent or solvent combination.

It is intended that the definition of any substituent or variable (e.g., R¹⁰, Z, n, etc.) at a particular location in a molecule be independent of its definitions elsewhere in that molecule. Thus, —N(R¹⁰)₂ represents —NHH, —NHCH₃, —NHC₂H₅, etc. It is understood that substituents and substitution patterns on the compounds of the instant invention can be selected by one of ordinary skill in the art to provide compounds that are chemically stable and that can be readily synthesized by techniques known in the art as well as those methods set forth below. available

Abbreviations used in the description of the chemistry and in the Examples that follow are:

| | |
|---|---|
| Ac₂O | Acetic anhydride; |
| Boc | t-Butoxycarbonyl; |
| DBU | 1,8-diazabicyclo[5.4.0]undec-7-ene; |
| DMAP | 4-Dimethylaminopyridine; |
| DME | 1,2-Dimethoxyethane; |
| DMF | Dimethylformamide; |
| EDC | 1-(3-dimethylaminopropyl)-3-ethyl-carbodiimide- |
| | hydrochloride |
| HOBT | 1-Hydroxybenzotriazole hydrate; |
| Et₃N | Triethylamine; |
| EtOAc | Ethyl acetate; |
| FAB | Fast atom bombardment; |
| HOOBT | 3-Hydroxy-1,2,2-benzotriazin-4(3H)-one; |
| HPLC | High-performance liquid chromatography; |
| MCPBA | m-Chloroperoxybenzoic acid; |
| MsCl | Methanesulfonyl chloride; |
| NaHMDS | Sodium bis(trimethylsilyl)amide; |
| Py | Pyridine; |
| TFA | Trifluoroacetic acid; |
| THF | Tetrahydrofuran. |

The compounds are useful in various pharmaceutically acceptable salt forms. The term “pharmaceutically acceptable salt” refers to those salt forms which would be apparent to the pharmaceutical chemist. i.e., those which are substantially non-toxic and which provide the desired pharmacokinetic properties, palatability, absorption, distribution, metabolism or excretion. Other factors, more practical in nature, which are also important in the selection, are cost of the raw materials, ease of crystallization, yield, stability, hygroscopicity and flowability of the resulting bulk drug. Conveniently, pharmaceutical compositions may be prepared from the active ingredients in combination with pharmaceutically acceptable carriers.

The inhibitors of KDR of the formulae I and II can be synthesized in accordance to Schemes 1-3 in addition to other standard manipulations such as ester hydrolysis, cleavage of protecting groups, etc., as may be known in the literature or exemplified in the experimental procedures.

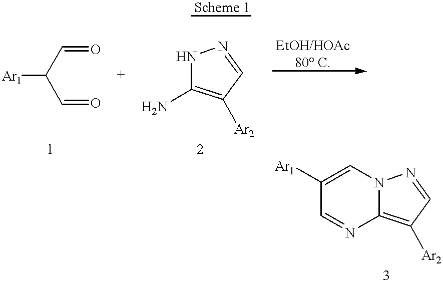

Generally, a method for the preparation of 3,6-diaryl pyrazolo(1,5-A)pyrimidines comprises mixing a commercially available malondialdehyde compound (1), with commercially available aminopyrazole (2) in an alcohol, such as ethanol, methanol, isopropanol, butanol and the like, said alcohol containing catalytic quantities of an acid, such as acetic acid, to yield (3), wherein Ar₁ and Ar₂, respectively, are R₄ and R₁, as illustrated above.

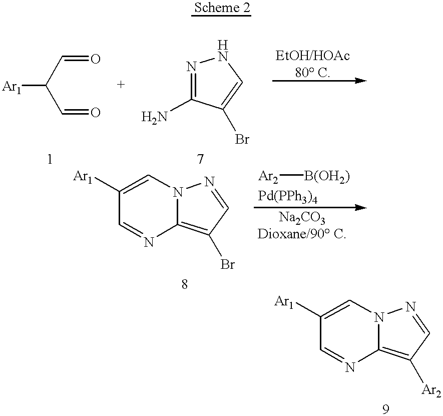

Scheme 2 depicts a means for making 3,6-diaryl pyrazolo(1,5-A)pyrimidines when the desired aminopyrazole is not commercially available. In a like manner to that described in scheme 1 compound (8) is obtained. Treatment of (8) with a boronic acid derivative in the presence of a palladium catalyst provides after workup the desired material (9). Ar₁ and Ar₂ are as described above.

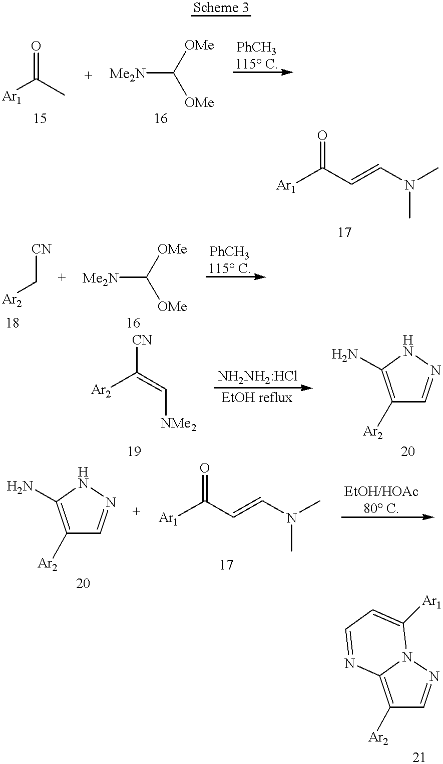

Scheme 3 ilustrates another method for the preparation of 3,7 diarylpyrazolo(1,5-A)pyrimidines. The comercially available ketone (15) and nitrile (18) are treated seperately with dimethylformamidedimethyl acetal (16) in refluxing toluene to give products (17) and (19) respectively. Compound (19) is then treated with hydrazinehydrochloride in refluxing ethanol to give the aminopyrazole (20). Compounds (17) and (20) and then treated with catalytic amounts of acetic acid in ethanol as described previously giving the desired of 3,7 diarylpyrazolo(1,5-A)pyrimidines (21). Ar₁ and Ar₂ are as described above.

The inhibitors of KDR of the formula III can be synthesized in accordance to Schemes 4-7 in addition to other standard manipulations such as ester hydrolysis, cleavage of protecting groups, etc., as may be known in the literature or exemplified in the experimental procedures.

As shown in Scheme 4, the quinoline reagent A can be synthesized by the general procedures taught in Marsais, F; Godard, A.; Queguiner, G. J. Heterocyclic Chem. 1989, 26, 1589-1594). Derivatives with varying substitution can be made by modifying this procedure and use of standard synthetic protocols known in the art. Also shown in Scheme 4 is the preparation of the indole intermediate D.

Scheme 5 illustrates one possible protocol for the coupling of the indole and quinolone intermediates to produce the desired compounds. Scheme 6 illustrates one possible synthetic route to the synthesis of a representative compound of the present invention, 3-(5-methoxy-1H-pyrrolo[2,3-c]pyridin-2-yl)-1H-quinolin-2-one.

Scheme 7 shows the synthesis of the iodo-naphthyridines and iodo-pyrido-pyridines. The resulting iodo compounds can then be coupled with appropriate indole boronic acid as taught in the other schemes to arrive at the desired product. The starting chloro-compounds can be prepared according to the method taught by D. J. Pokomy and W. W. Paudler in J. Org. Chem. 1972, 37, 3101.

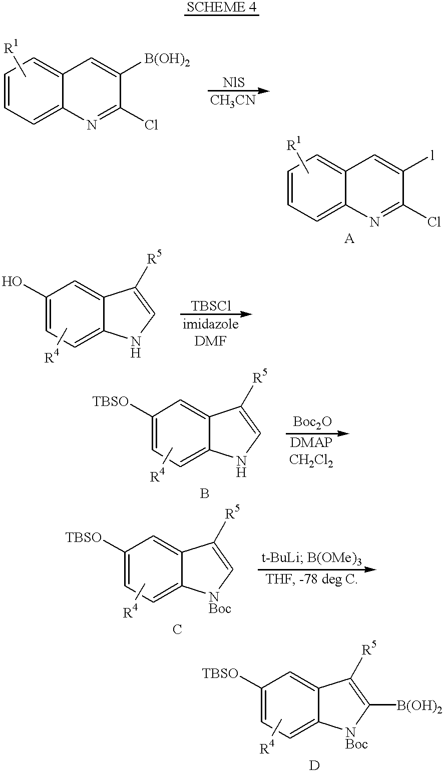

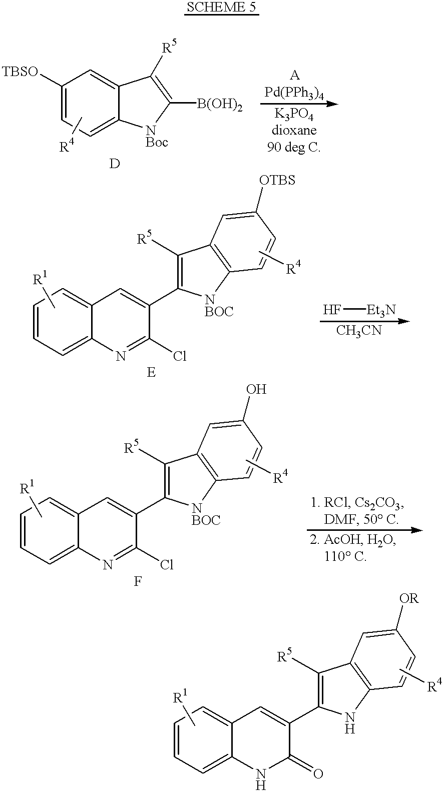

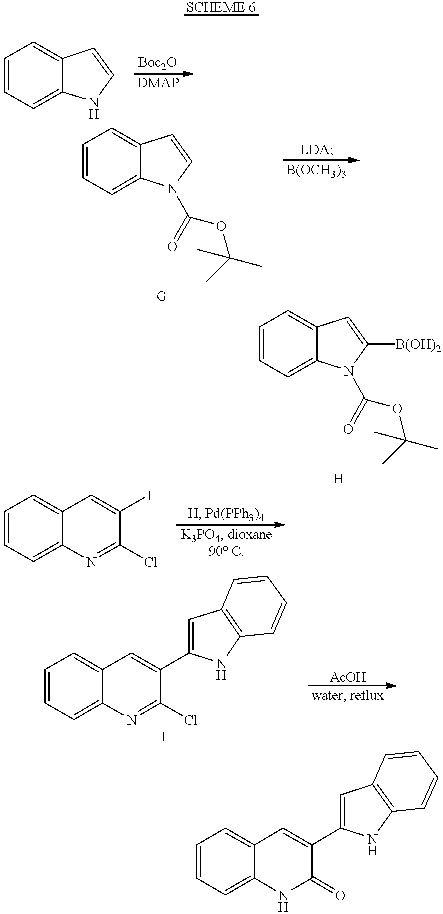

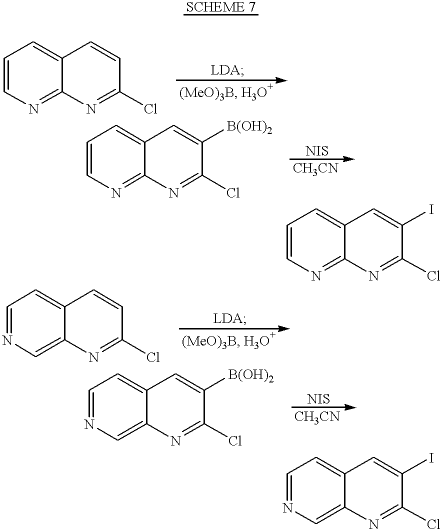

The inhibitors of KDR of the formula IV can be synthesized in accordance to Schemes 8-11 in addition to other standard manipulations such as ester hydrolysis, cleavage of protecting groups, etc., as may be known in the literature or exemplified in the experimental procedures.

As shown in Scheme 8, the quinoline reagent 1-2 can be synthesized by the general procedures taught in Marsais, F; Godard, A.; Queguiner, G. J. Heterocyclic Chem. 1989, 26, 1589-1594). Derivatives with varying substitution can be made by modifying this procedure and use of standard synthetic protocols known in the art. Intermediate 1-2 is then coupled with the appropriate N-protected pyrollo-compound, structure 1-4, to produce a chlorinated intermediate of structure 1-5. Heating of 1-5 in aqueous acetic acid produces the desired de-chlorinated product, 1-6. Scheme 9 shows an example using this route to arrive at a [3,2]-pyridno-pyrole, 2-3.

As shown in Scheme 10, the α-alkyloxy pyridino-pyroles 3-1 can be converted to the corresponding pyrimidinone analogs 3-2 by heating with aqueous HBr. Alternatively, the pyrimidinone analogs can be synthesized via the N-oxide intermediates 4-2 as shown in Scheme 11.

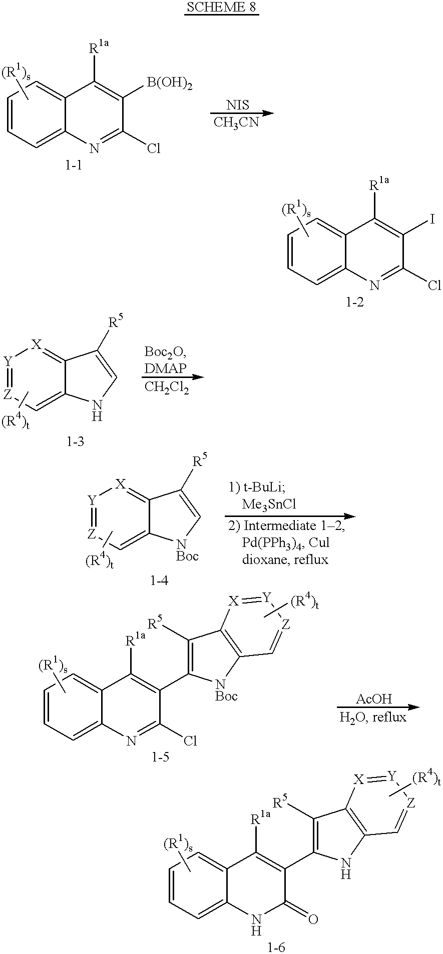

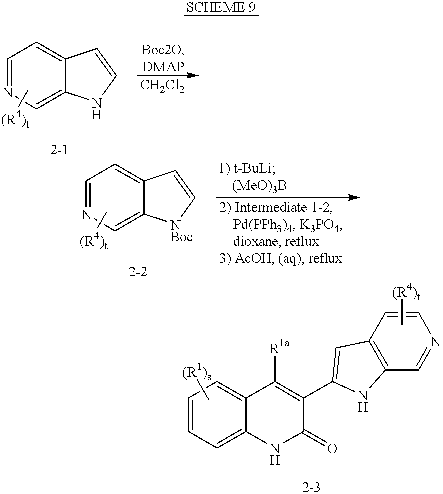

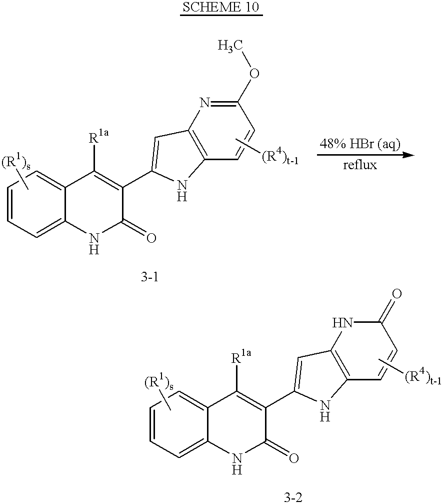

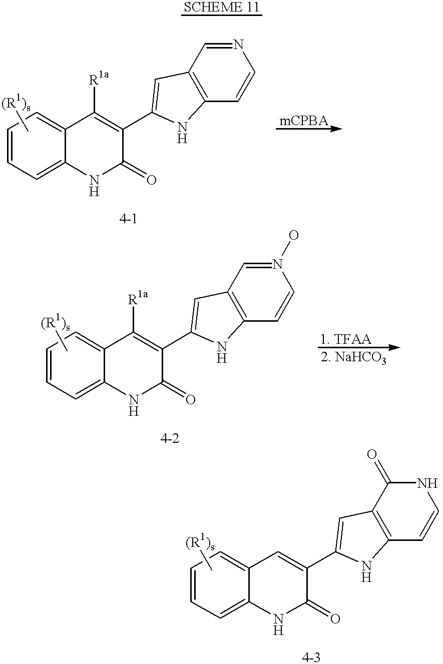

The PSA conjugates of formulae IX, XI and XIII can be synthesized in accordance with Schemes 12-16, in addition to other standard manipulations such as ester hydrolysis, cleavage of protecting groups, etc., as may be known in the literature or exemplified in the experimental procedures.

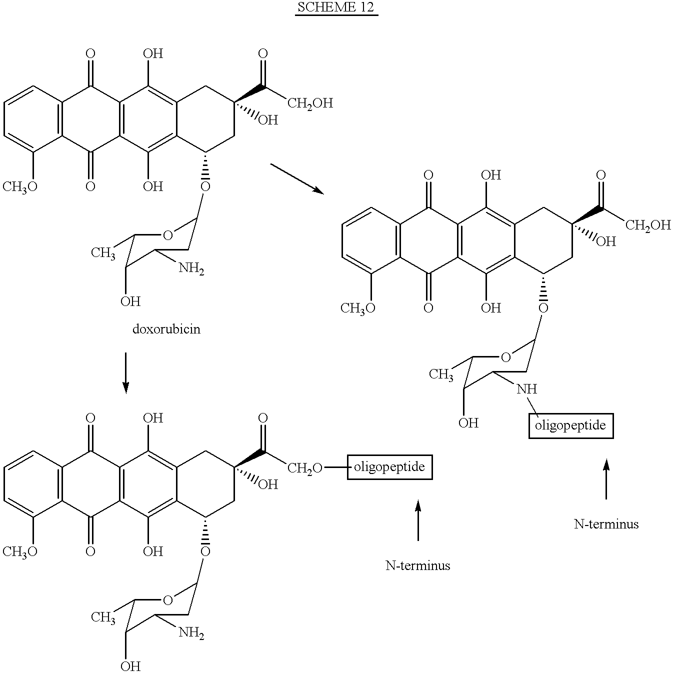

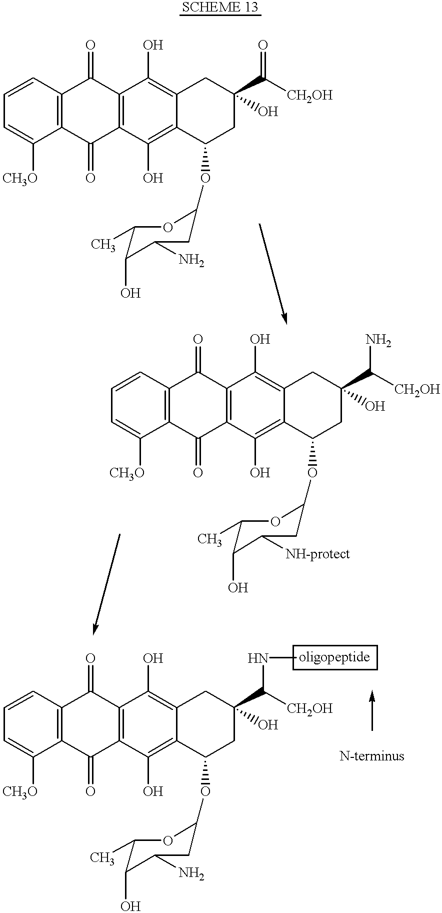

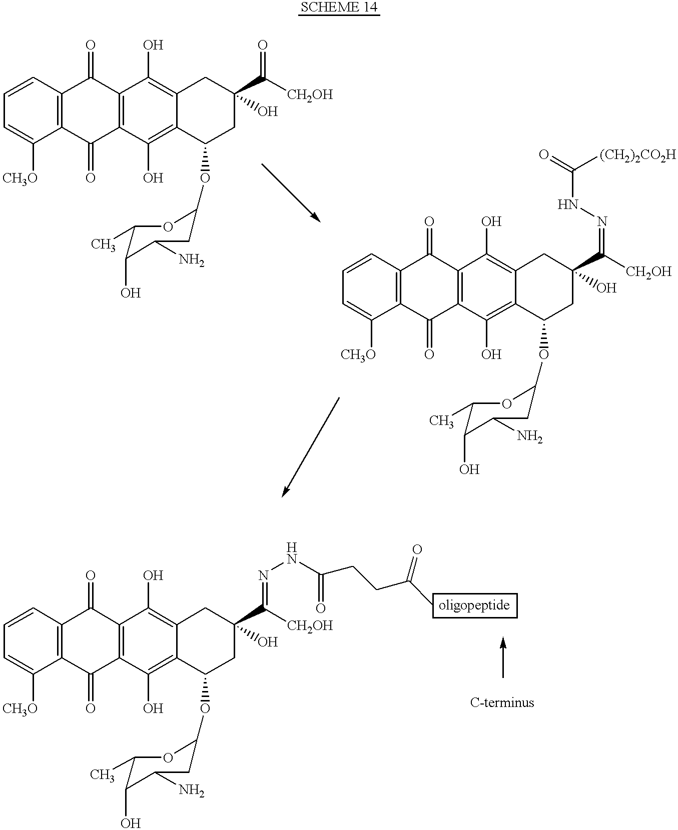

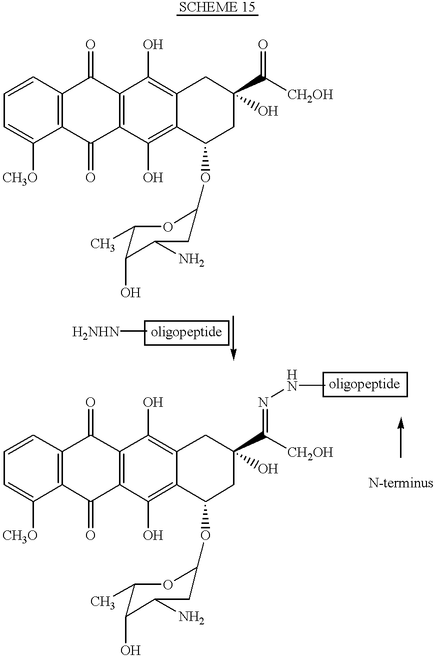

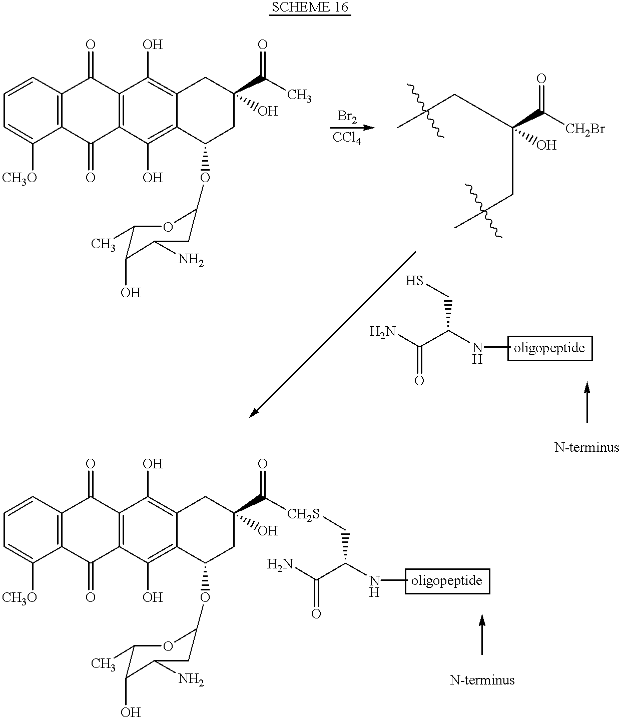

Scheme 17 illustrates preparation of conjugates utilized in the instant method of treatment wherein the oligopeptides are combined with the vinca alkaloid cytotoxic agent vinblastine, such as the compounds of the formula X. Attachment of the N-terminus of the oligopeptide to vinblastine is illustrated (S. P. Kandukuri et al. J. Med. Chem. 28:1079-1088 (1985)).

Scheme 18 illustrates preparation of conjugates of the oligopeptides of the instant invention and the vinca alkaloid cytotoxic agent vinblastine wherein the attachment of vinblastine is at the C-terminus of the oligopeptide. The use of the 1,3-diaminopropane linker is illustrative only; other spacer units between the carbonyl of vinblastine and the C-terminus of the oligopeptide are also envisioned. Furthermore, Scheme 18 illustrates a synthesis of conjugates wherein the C-4-position hydroxy moiety is reacetylated following the addition of the linker unit. Applicants have discovered that the desacetyl vinblastine conjugate is also efficacious and may be prepared by eliminating the steps shown in Scheme 18 of protecting the primary amine of the linker and reacting the intermediate with acetic anhydride, followed by deprotection of the amine. Conjugation of the oligopeptide at other positions and functional groups of vinblastine may be readily accomplished by one of ordinary skill in the art and is also expected to provide compounds useful in the treatment of prostate cancer.

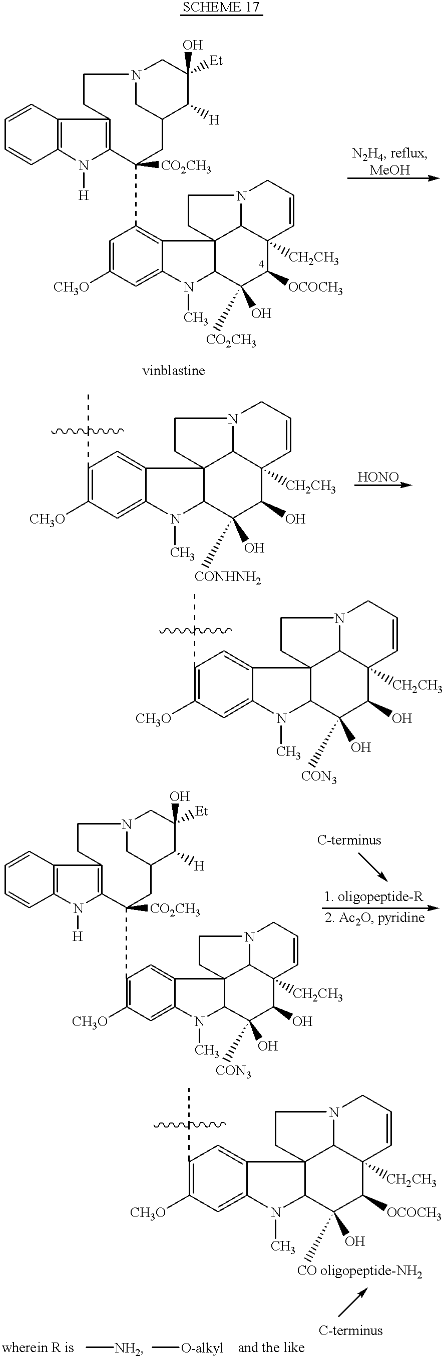

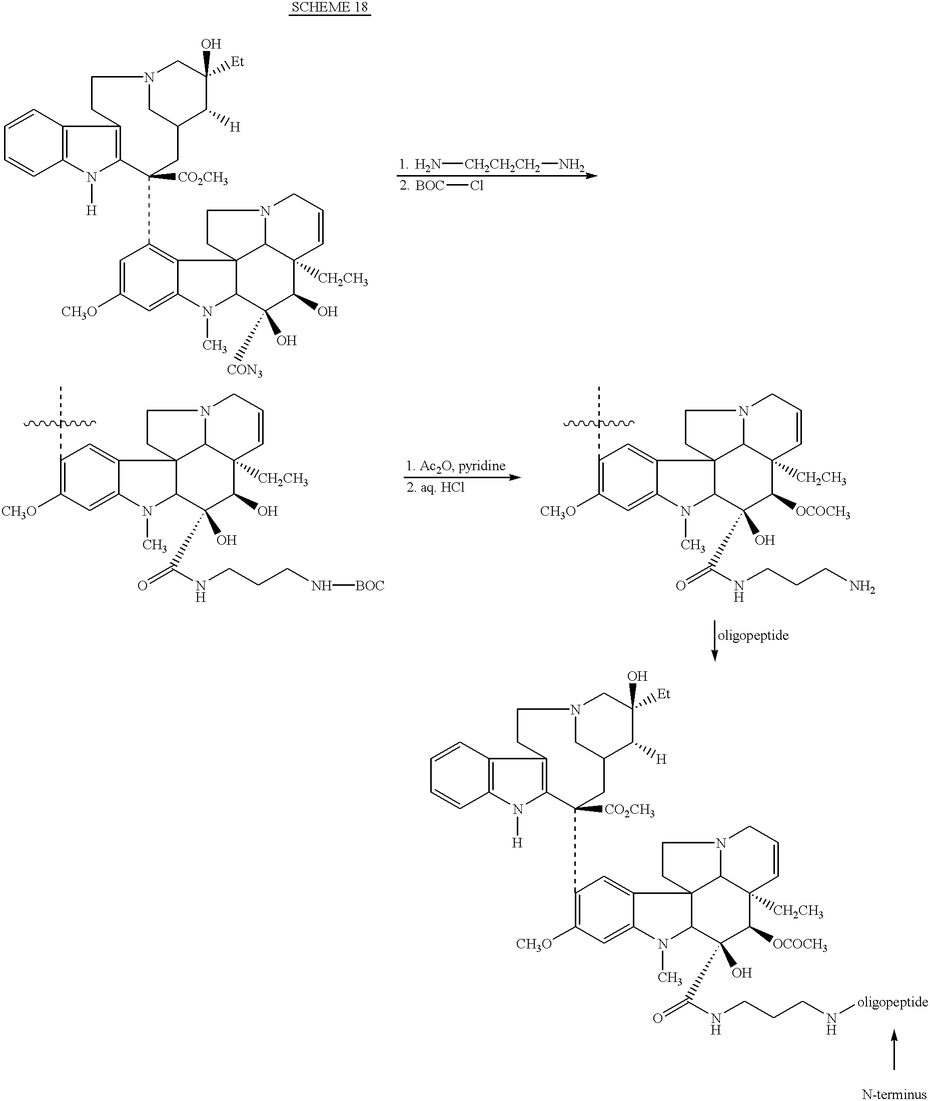

The PSA conjugates of formula XI and XIII can be synthesized in accordance with Schemes 19-23, in addition to other standard manipulations such as ester hydrolysis, cleavage of protecting groups, etc., as may be known in the literature or exemplified in the experimental procedures.

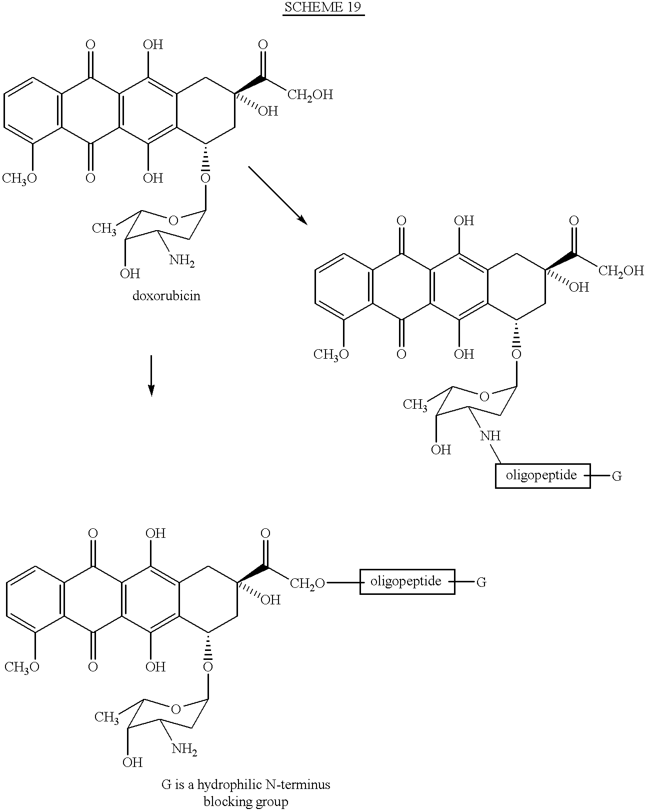

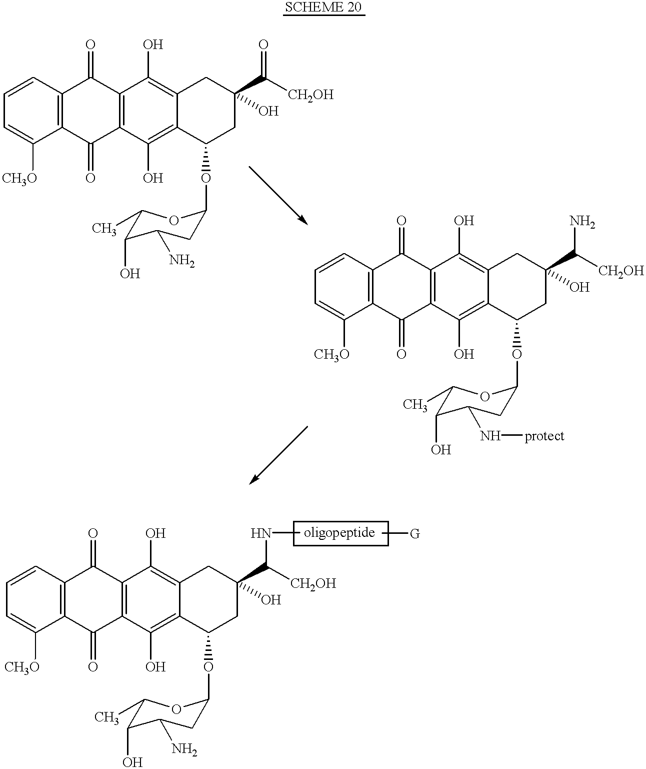

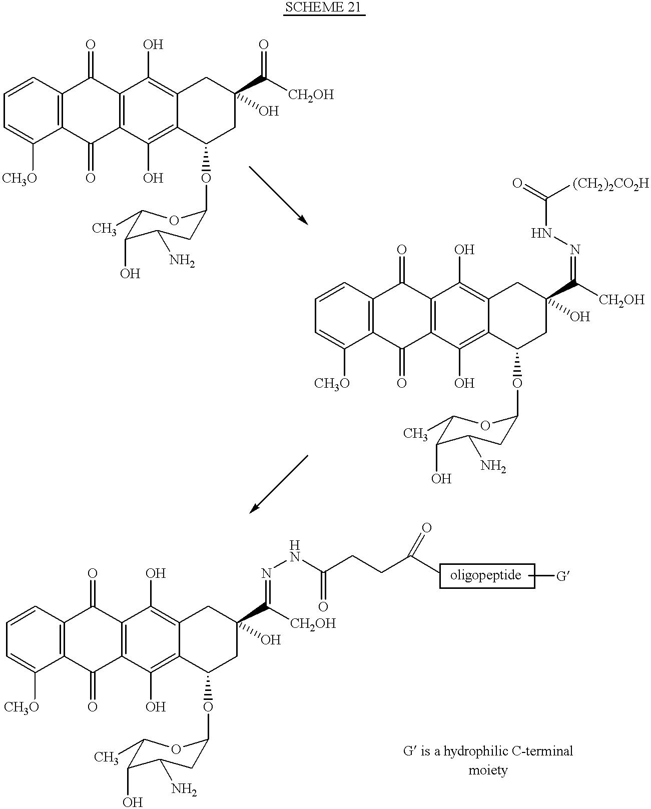

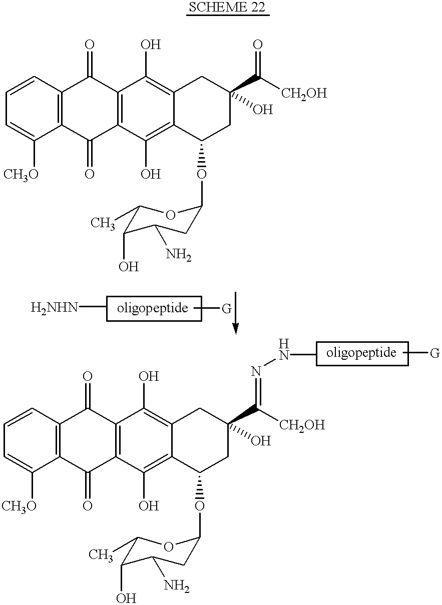

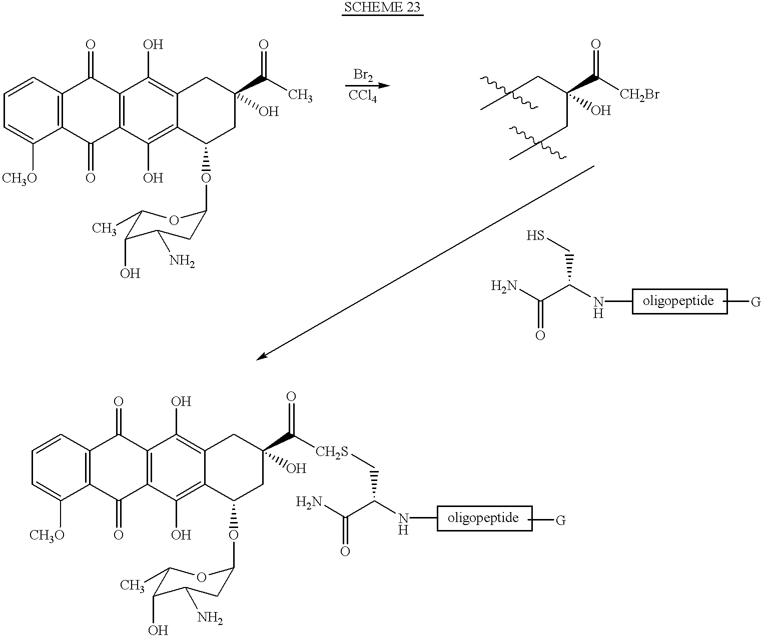

Scheme 24 illustrates preparation of PSA conjugates of the formula XIV wherein the attachment of vinblastine is at the C-terminus of the oligopeptide. Furthermore, Scheme 24 illustrates a synthesis of conjugates wherein the C-4-position hydroxy moiety is reacetylated following the addition of the linker unit. Applicants have discovered that the desacetyl vinblastine conjugate is also efficacious and may be prepared by eliminating the steps shown in Scheme 24 of protecting the primary amine of the linker and reacting the intermediate with acetic anhydride, followed by deprotection of the amine. Conjugation of the oligopeptide at other positions and functional groups of vinblastine may be readily accomplished by one of ordinary skill in the art and is also expected to provide compounds useful in the treatment of prostate cancer.

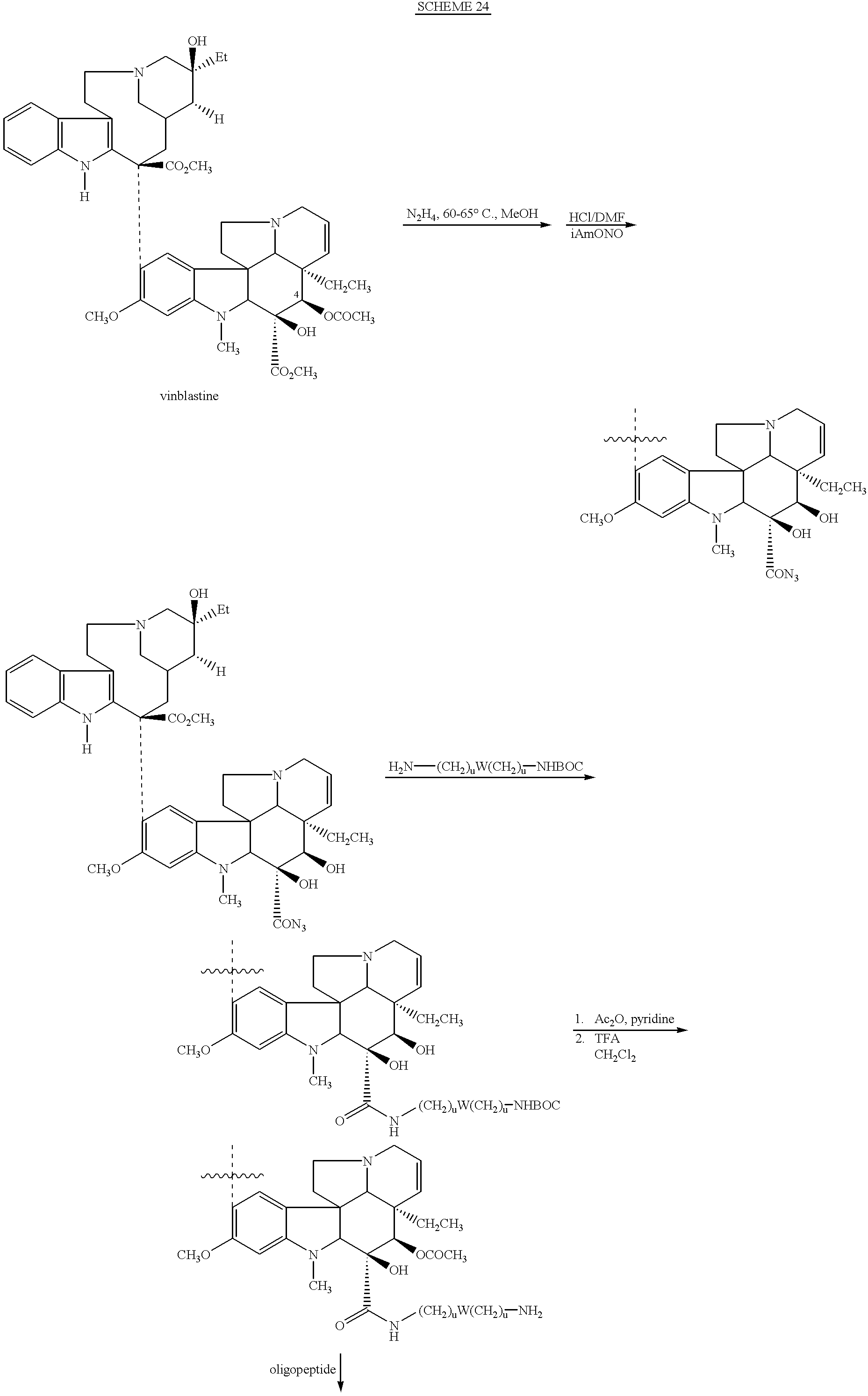

The PSA conjugates of formula XV can be synthesized in accordance with Schemes 25-26, in addition to other standard manipulations such as ester hydrolysis, cleavage of protecting groups, etc., as may be known in the literature or exemplified in the experimental procedures.

Reaction Scheme 25 illustrates preparation of conjugates of the oligopeptides of the instant invention and the vinca alkaloid cytotoxic agent vinblastine wherein the attachment of the oxygen of the 4-desacetylvinblastine is at the C-terminus of the oligopeptide. While other sequences of reactions may be useful in forming such conjugates, it has been found that initial attachment of a single amino acid to the 4-oxygen and subsequent attachment of the remaining oligopeptide sequence to that amino acid is a preferred method. It has also been found that 3,4-dihydro-3-hydroxy-4-oxo-1,2,3-benzotriazine (ODHBT) may be utilized in place of HOAt in the final coupling step.

Reaction Scheme 26 illustrates preparation of conjugates of the oligopeptides of the instant invention wherein a hydroxy alkanolyl acid is used as a linker between the vinca drug and the oligopeptide.

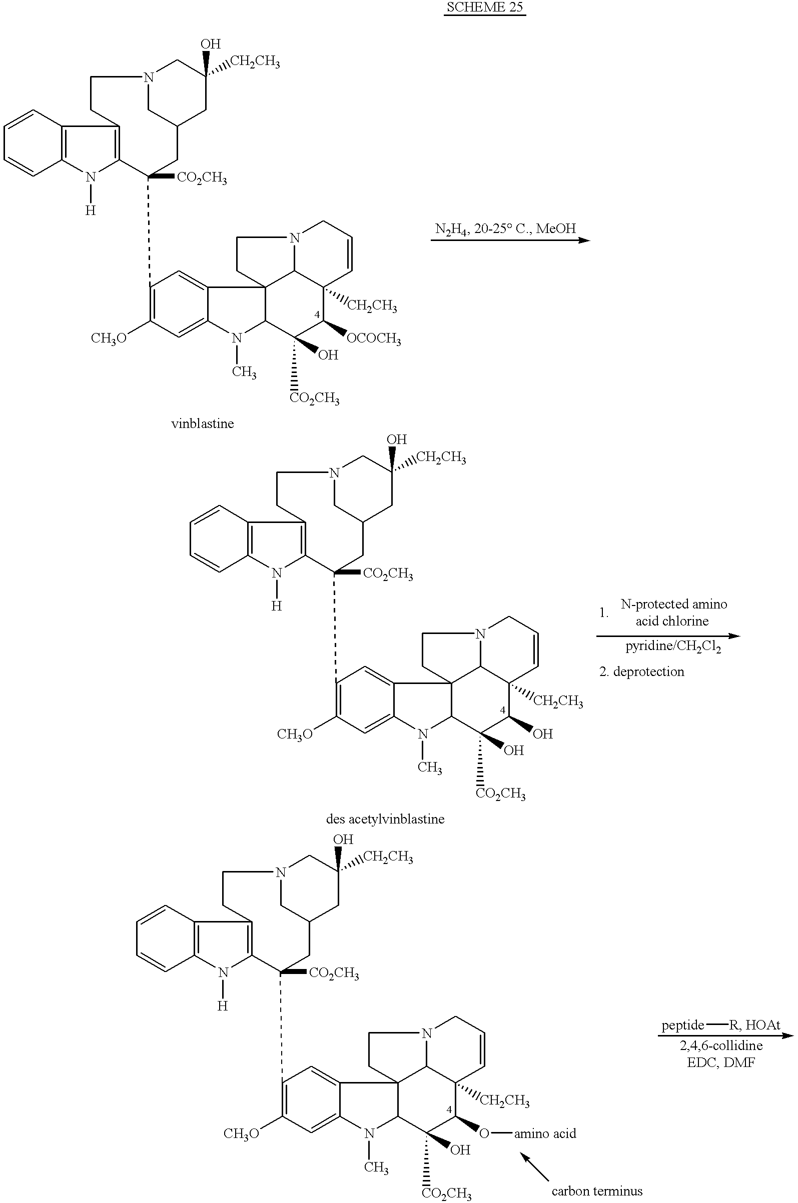

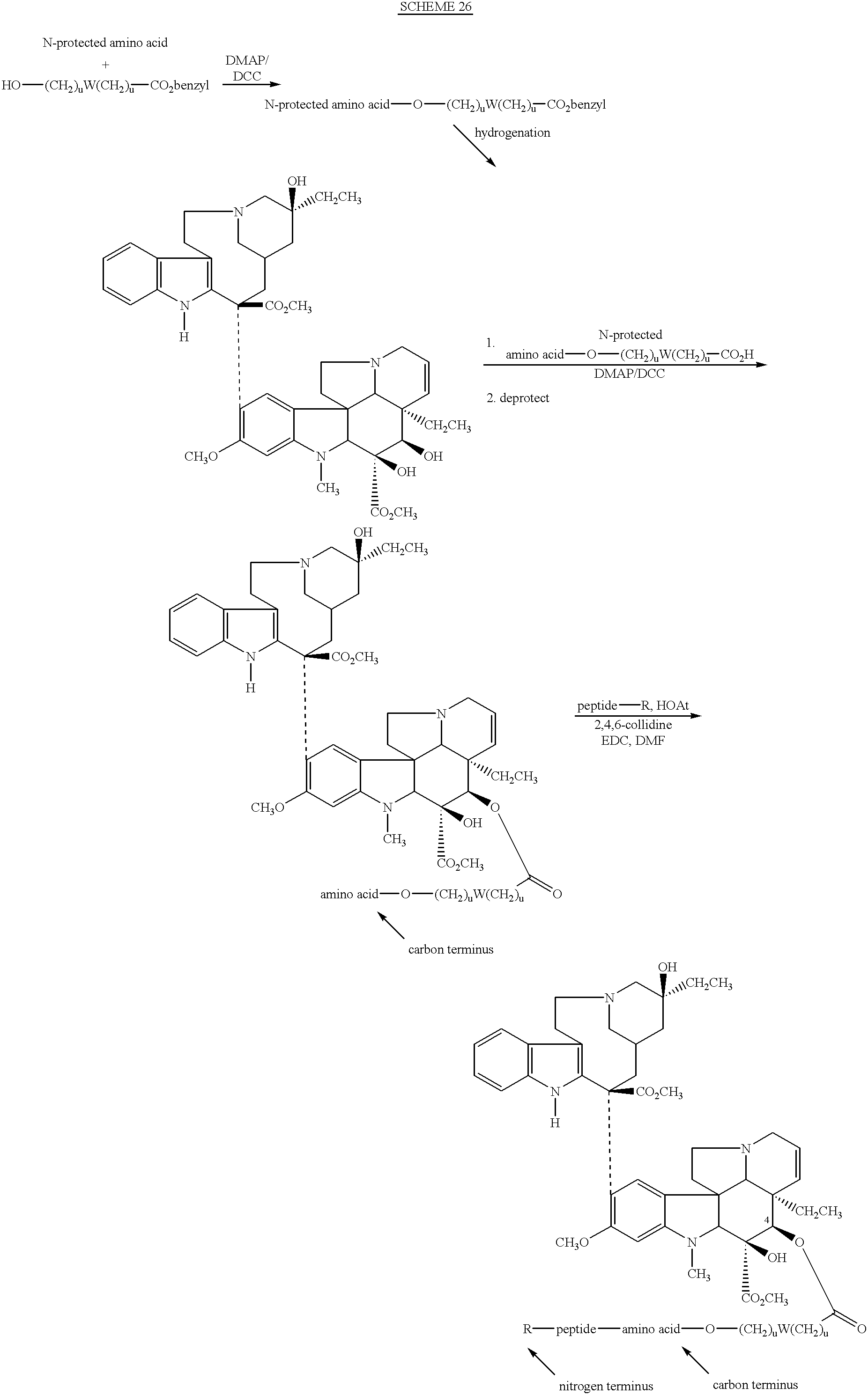

### EXAMPLES

Examples provided are intended to assist in a further understanding of the invention. Particular materials employed, species and conditions are intended to be further illustrative of the invention and not limitative of the reasonable scope thereof.

The standard workup referred to in the examples refers to solvent extraction and washing the organic solution with 10% citric acid, 10% sodium bicarbonate and brine as appropriate. Solutions were dried over sodium sulfate and evaporated in vacuo on a rotary evaporator.

### Example 1

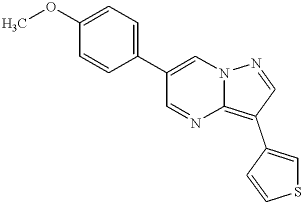

### 3-(3-thienyl)-6-(4-methoxyphenyl)pyrazolo(1,5-A)pyrimidine(5)

Step 1

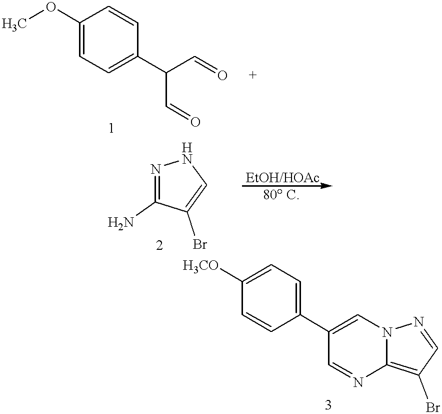

A solution of 1 (713 mg, 4.0 mmol) and commercially availaible 2 (648 mg, 4.0 mmol), discussed above in ethanol (20 mL) was heated at 75° C. for 4 h. The resulting white suspension was as decribed in example 1 for 4 hours, then cooled to 20° C., filtered, and washed with methanol (3×5 mL) to provide Intermediate 3 as a white powder (mp=168-170° C.):¹H NMR (CDCl₃)δ8.79 (d, 1 H, J=2.2 Hz), 8.74 (d, 1 H, J=2.2 Hz), 8.12 (s, 1 H), 7.51 (d, 2 H, J=8.8 Hz), 7.05 (d, 2 H, J=8.8 Hz), 3.88 (s, 3 H).

Step 2

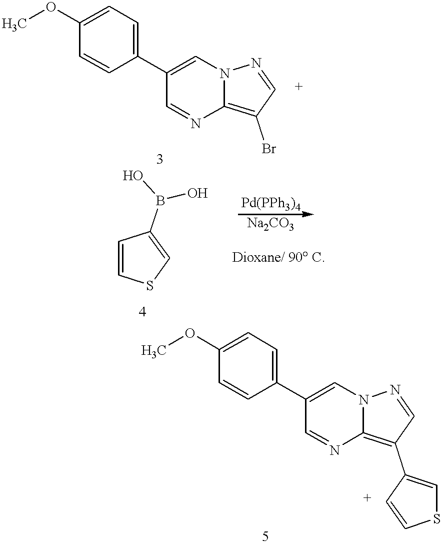

A suspension of intermediate (3), prepared as described in Step 1 (250 mg, 0.82 mmol), thiophene-3-boronic acid (4) (158 mg, 1.24 mmol), and aqueous sodium carbonate (2 M, 1 mL) in dioxane (5 mL) was de-gassed by evacuating and backflushing with argon (3×). Tetrakis(triphenyl-phosphine) palladium (20 mg, 0.017 mmol) was added and the reaction mixture was de-gassed again. The argon filled flask was then submerged in an oil bath pre-heated to 90° C. and was heated at that temperature for 16 h. After cooling to 20° C., the yellow precipitate which formed was collected by filtration and was washed with methanol (3×5 mL) to provide the title compound (5) as a yellow powder (mp=191-193° C.): ¹H NMR (CDCl₃) δ8.79 (d, 1 H, J=2.4 Hz), 8.76 (d, 1 H, J=2.2 Hz), 8.37 (s, 1 H), 7.90 (dd, 1 H, J=2.9, 1.3 Hz), 7.70 (dd, 1 H, J=4.9, 1.2 Hz), 7.54 (d, 2 H, J=8.8 Hz), 7.43 (d, 1 H, J=4.9, 2.9 Hz), 7.06 (d, 2 H, J=8.8 Hz), 3.88 (s, 3H).

### Example 2

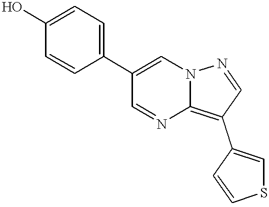

### 3-(3-thienyl)-6-(4-hydroxyphenyl)pyrazolo(1,5-A)pyrimidine (6)

Method A

Ethanethiol (30 mg, 36 μL) was added dropwise over 1 min to a suspension of sodium hydride (23 mg, 0.98 mmol) in dry DMF (2 mL) under argon. After 15 min, 3-(3-thienyl)-6-(4-methoxyphenyl)pyrazolo(1,5-A)pyrimidine (5), prepared as described in Example 1 (50 mg, 0.16 mmol) was added and the reaction mixture was heated at 150° C. for 1.5 h. The resulting brown solution was cooled, poured into water (25 mL) and washed with ethyl acetate (2×25 mL). The combined organics were dried (Na₂SO₄), concentrated, and purified by flash chromatography (40% EtOAc/Hexanes) to provide the title compound as a yellow solid[R_{f}=0.12 (40% EtOAc/Hexanes)]: ¹H NMR (CD₃OD) δ8.96 (d, 1 H, J=2.4 Hz), 8.85 (d, 1 H, J=2.2 Hz), 8.44 (s, 1 H), 7.94 (dd, 1 H, J=2.9, 1.2 Hz), 7.74 (dd, 1 H, J=4.9, 1.2 Hz), 7.56 (d, 2 H, J=8.8 Hz), 7.46 (dd, 1H, J=4.9, 2.9 Hz), 6.94 (d, 2H, J=8.6 Hz).

Method B

A mixture of (5) (10.3 g, 33.5 mmol, 1 equiv), prepared as described in Example 1, and lithium iodide (28.2 g, 211 mmol, 6.30 equiv) was heated in 2,4,6-collidine at 180 deg C. for 28 h. The reaction mixture was cooled, then partitioned between aqueous 3 N HCl solution and ethyl acetate (4×500 mL). The combined organic layers were dried over sodium sulfate and concentrated. The residual solid was suspended in methanol (300 ml), then filtered and air dried to give a 3:1 mixture of the title compound and 5, respectively, as a yellow solid.

### Example 3

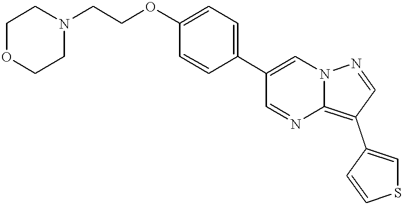

### 3-(3-thienyl)-6-(4-(2-(4-morpholinyl)ethoxy)phenyl)pyrazolo(1,5-A)pyrimidine (7)

A solution of 3-(3-thienyl)-6-(4-hydroxyphenyl)-pyrazolo(1,5-A)pyrimidine (6), prepared as described in Example 2 (11 mg, 0.038 mmol), cesium carbonate (37 mg, 0.11 mmol), N-(2-chloroethyl)morpholine hydrochloride (7 mg, 0.11 mmol), and sodium iodide (0.013 mmol) in DMF (3 mL) was heated at 60° C. under argon for 16 h. The reaction mixture was then poured into water (25 mL) and washed with ethyl acetate (2×25 mL). The combined organics were dried (Na₂SO₄), concentrated, and purified by flash chromatography [50% Hexanes/CHCl₃(NH₃)] to give the title compound as a yellow solid [mp=149-151° C., R_{f}=0.39 (100% CHCl₃(NH₃))]: ¹H NMR (CDCl₃) δ8.77 (d, 1 H, J=2.2 Hz), 8.75 (d, 1H, J=2.2 Hz), 8.36 (s, 1 H), 7.90 (dd, 1 H, J=2.9, 1.3 Hz), 7.69 (dd, 1 H, J=4.9, 1.3 Hz), 7.52 (d, 2 H, J=8.8 Hz), 7.43 (d, 1 H, J=4.9, 2.9 Hz), 7.06 (d, 2 H, J=8.8 Hz), 4.18 (t, 2 H, J=5.7 Hz), 3.76 (t, 4 H, J=4.6 Hz), 2.85 (t, 2 H, J=5.7 Hz), 2.61 (t, 4 H, J=4.6 Hz); Anal Calcd. for C₂₂H₂₂N₄O₂S: C, 65.00; H, 5.46; N, 13.78. Found C, 64.98; H, 5.55; N, 14.02.

### Example 4

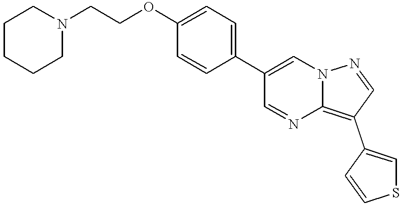

### 6-[4-(2-piperidin-1-yl-ethoxy)-phenyl]-3-thiophen-3-yl-pyrazolo[1,5-a]pyrimidine (8)

Sodium hydride (95%, 720 mg, 28.5 mmol, 2.10 equiv) was carefully added to a rapidly stirred solution of a 3:1 mixture of 6 and 5 (4.0 g, 13.6 mmol, 1 equiv), prepared according to Example 2 (Method B), in N,N-dimethylformamide (50 mL) at 23 deg C. After 5 min, N-(2-chloroethyl)piperidine hydrochloride (2.76 g, 15.0 mmol, 1.10 equiv) was added and the resulting mixture was immersed in a pre-heated (60 deg C.) oil bath. The reaction mixture was held at 60 deg C. for 30 min, then partitioned between water (300 mL) and ethyl acetate (2×200 mL). The combined organic layers were dried over sodium sulfate and concentrated. The residue was purified by flash column chromatography (dichloromethane initially, grading to 10% methanol in dichloromethane) to give the title compound as a yellow solid (mp=141-143° C.). ¹H NMR (CDCl₃) δ8.79 (d, 1 H, J=2.2 Hz), 8.76 (d, 1 H, J=2.2 Hz), 8.36 (s, 1 H), 7.90 (dd, 1 H, J=2.9, 1.3 Hz), 7.70 (dd, 1 H, J=4.9, 1.3 Hz), 7.52 (d, 2 H, J=8.8 Hz), 7.43 (d, 1 H, J=4.9, 2.9 Hz), 7.06 (d, 2 H, J=8.8 Hz), 4.18 (t, 2 H, J=6.0 Hz), 2.82 (t, 2 H, J=6.0 Hz), 2.54 (br m, 4H), 1.63 (br m, 4H), 1.47 (br m, 2H); HRMS (electrospray FT/ICR) calcd for C23 H25N4OS [M+H]⁺405.1743, found 405.1740; anal calcd for C₂₃H₂₄N₄OS: C, 68.29; H, 5.98; N, 13.85, found C, 69.10; H, 5.94; N, 13.98.

### Example 5

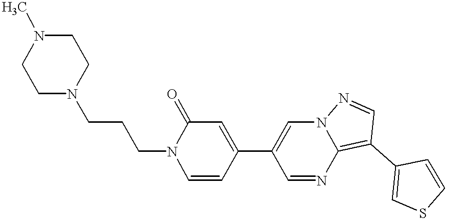

### 1-[3-(piperidin-1-yl)-propyl)]-4-(3-thiophen-3-yl-pyrazolo[1,5-a]pyrimidin-6-yl)-1H-pyridin-2-one (9)

Step 1: 6-Bromo-3-thiophen-3-yl-pyrazolo[1,5-a]pyrimidine(5-3)

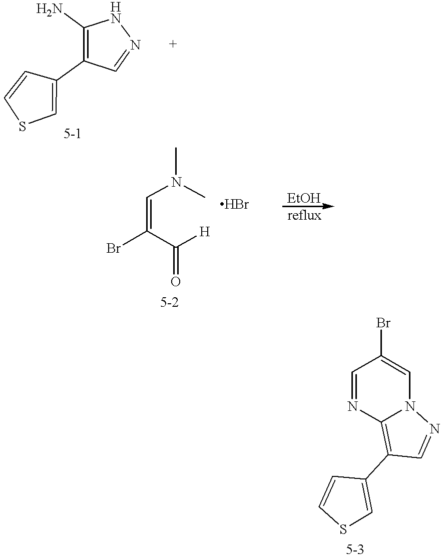

A solution of 5-1(J. Heterocycl. Chem. (1995), 32(1), 291-8) (4.3 g, 26 mmol.) and 5-2 (Helv. Chim. Acta (1969), 52(8), 2641-57) (7.75 g, 29.9 mmol) in ethanol(100 ml) was refluxed for 2 hr. The reaction mixture was cooled to room temp. and the product (5-3) was collected by filtration. ¹H NMR(400 MHz, CDCl₃) δ8.83(dd, 1H, J=5, 2 Hz), 8.52 (dd, 1H, J=5, 2 Hz), 8.33 (s, 1H), 7.86 (dd, 1H, J=3, 1 Hz), 7.66 (dd, 1H, J=6, 4 Hz), 7.42 (dd, 1H, J=5, 3 Hz).

Step 2: 4-Bromo-2-methoxypyridine(5-5)

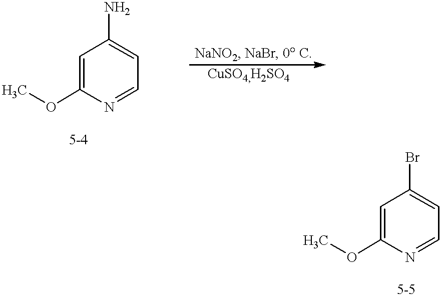

A saturated solution of NaNO₂(817 mg, 11.5 mmol) cooled to 0° C. was added dropwise to a stirred suspension of 5-4(J. Heterocycl. Chem. (1985), 22(1), 145-7) (1.2 g, 10 mmol) NaBr(391 mg, 38 mmol) and CuSO₄(750 mg, 29 mmol) in 9 M H₂SO₄(3 ml) cooled to −5° C. in ice/salt water bath. The reaction was stirred 20 min at −5° C. and allowed to warm to rt before it was poured unto ice and made basic with 50% NaOH. The resulting mixture was extracted into ethyl acetate. The extracts were combined, dried over MgSO₄ and concentrated to give a tan oil which was chromatographed on silica gel. Elution with 50% Hexanes/CH₂Cl₂ to 100% CH₂Cl₂ provided 5-5 as a colorless gum. ¹H NMR(400 MHz, CDCl) δ7.98(d, 1H, J=6 Hz), 7.02 (dd, 1H, J=6, 2 Hz), 6.94 (d, 1H, J=2 Hz), 3.92 (s, 3H).

Step 3: 2-Methoxy-4-(4,4,5,5-tetramethyl-[1,3,2]dioxaborolan-2-yl)-pyridine (5-7)

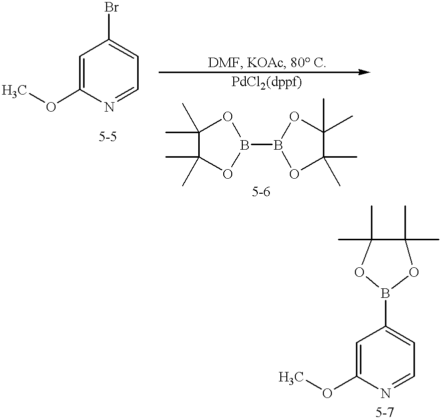

Bis(pinacolato)diboron 5-6 (983 mg, 3.8 mmol), 5-5(662 mg, 3.5 mmol) and potassium acetate(1036 mg, 10.5 mmol) were added to DMF(5 ml). The reaction was deoxygenated before PdCl₂(dppf)(144 mg, 0.176 mmol) was added. The reaction was heated at 80° C. for 4 hr. The DMF was removed at 40° C. and the residue was partitioned between ethyl acetate and sat. NaHCO₃. The organic layer was washed with brine, dried over MgSO₄ and concentated to give 5-7 as a brown oil. ¹H NMR(400 MHz, CDCl₃) δ8.17(d, 1H, J=5 Hz), 7.17 (d, 1H, J=5 Hz), 7.12 (bs, 1H), 3.92 (s, 3H), 1.26 (s, 12H)

Step 4: 6-(2-Methoxypyridin-4-yl)-3-thiophen-3-yl-pyrazolo[1-5-a]pyrimidine(5-8)

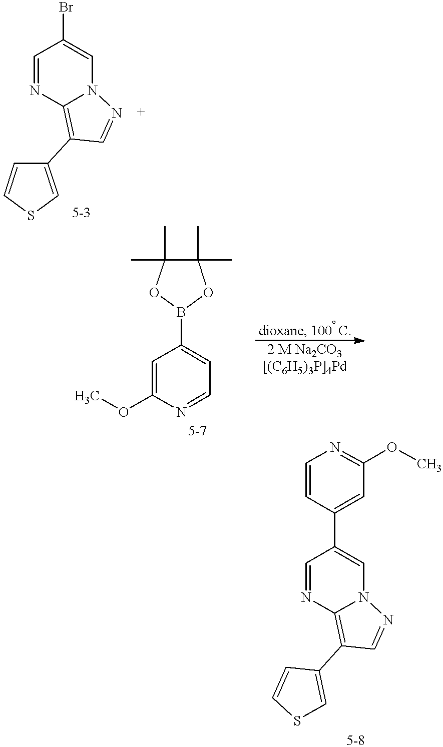

A mixture of 5-3(389 mg, 1.4 mmol), 5-7(653 mg, 2.78 mmol) 2 M Na₂CO₃(1.5 ml) in dioxane(5 ml) was deoxygenated before the tetrakis(triphenylphosphine)palladium(O) (80 mg, 0.069 mmol) was added. The reaction was heated at 100° C. under argon for 16 hr. The cooled reaction mixture was partitioned between ethyl acetate and water. The organic layer was washed with brine, dried over MgSO₄ and concentrated to give a yellow solid which was chromatographed on silica gel. Elution with CH₂Cl₂ to 10% EtOAc/CH₂Cl₂ gave 5-8 as a yellow solid. ¹H NMR(400 MHz, CDCl₃) δ8.89(d, 1H, J=2 Hz), 8.80 (d, 1H, J=2 Hz), 8.43 (s, 1H), 8.31 (d, 1H, J=5 Hz), 7.91 (m, 1H), 7.71 (d, 1H, J=2 Hz), 7.44 (dd, 1H, J=5, 2 Hz), 7.12 (d, 1H, J=4 HZ), 6.99 (s, 1H), 4.02 (s, 3H)

Step 5: 4-(3-Thiophen-3-yl-pyrazolo[1,5-a]pyrimidin-6-yl)-1H-pyridin-2-one(5-9)

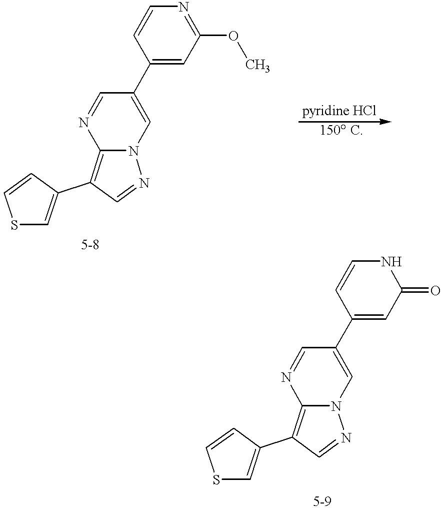

Pyridine hydrochloride(1.15 g, 10 mmol) and 5-8(0.154 g, 0.5 mmol) were mixed and heated at 150° C. for 15 min. The reaction was cooled and diluted with water to give 5-9 as a yellow solid. 1H NMR(400 MHz, DMSO) δ11.75(s, 1H), 9.59 (d, 1H, J=2 Hz), 9.03 (d, 1H, J=2 Hz), 8.78 (d, 1H, J=3 Hz), 8.03 (d Hz), 7.84 (dd, 1H, J=4, 1 Hz), 7.67 (dd, 1H, J=5, 3 Hz), 7.51 (d, 1H, J=6 HZ), 6.91 (d, 1H, J=2 Hz), 6.73 (dd, 1H, J=7, 2 Hz).

Step 6: 1-[3-(4-Methylpiperazin-1-yl)propyl]-4-(3-thiophen-3-ylpyrazolo[1,5-a]pyrimidin-6-yl)-1H-pyrin-2-one(9)

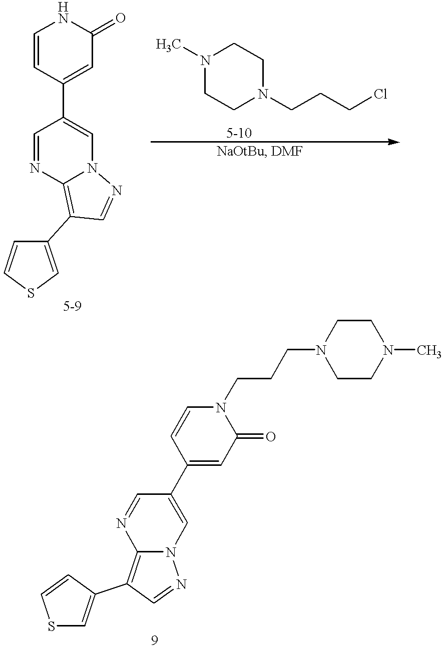

5-9(2.3 g,7.8 mmol), 51-10(2.07 g, 11.7 mmol) and sodium tert-butoxide(0.83 g, 8.6 mmol) were added to DMF(600 ml) and the reaction warmed at 70° C. for 18 hr. The DMF was removed at 40° C. and the residue was partitioned between ethyl acetate and water. The organic layer was washed with brine, dried over MgSO₄ and concentrated to give a yellow solid which was chromatographed on silica gel. Elution with 5% NH₃-EtOH/CH₂Cl₂ to 10% NH₃-EtOH/CH₂Cl₂ gave the title compound (9) as a yellow solid. ¹H NMR(400 MHz, CD₃OD) δ9.30 (d, 1H, J=2 Hz), 8.91 (d, 1H, J=2 Hz), 8.57 (s, 1H), 7.97 (dd, 1H, J=3, 1 Hz), 7.80 (d, 1H, J=7 Hz), 7.77 (dd, 1H, J=5, 1 Hz), 7.48 (dd, 1H, J=5, 3 Hz), 6.95 (d, 1H, J=2 Hz), 6.82 (dd, 1H, J=7, 2 Hz), 4.09 (t, 2H, J=7 Hz), 2.62-2.40 (m, 10 H), 2.03 (s, 3H), 1.99 (m, 2H).

### Example 6

### 3-[5-(2-piperidin-1-yl-ethoxy)-1H-indol-2-yl]-1H-quinolin-2-one (10)

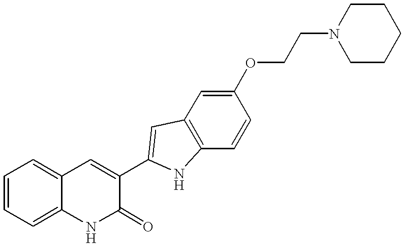

Step A: Preparation of 2-chloro-3-iodo-quinoline (Intermediate A)

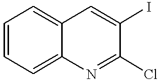

A suspension of 3-(2-chloro)-quinolineboronic acid (5.05 g, 24.3 mmol, 1 equiv, prepared by the method of Marsais, F; Godard, A.; Queguiner, G. J. Heterocyclic Chem. 1989, 26, 1589-1594) and N-iodosuccinimide (5.48 g, 24.4 mmol, 1.00 equiv) in acetonitrile (300 mL) was stirred at 23° C. in the dark for 20 h. The reaction mixture was concentrated to dryness and the resulting yellow solid was partitioned between saturated aqueous sodium bicarbonate solution and dichloromethane. The organic layer was washed with water, then dried over magnesium sulfate and concentrated to give 2-chloro-3-iodo-quinoline (intermediate A) as a pale yellow solid. ¹H NMR (400 MHz, CDCl₃) δ8.67 (s, 1H), 7.99 (br d, 1H, J=8.4 Hz), 7.75 (br t, 1H, J=7.7 Hz), 7.72 (br d, 1H, J=7.8 Hz), 7.57 (br t, 1H, J=7.6 Hz).

Step B: Preparation of 5-(tert-butyl-dimethyl-silanyloxy)-1H-indole (Intermediate B)

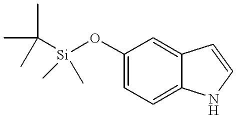

A solution of 5-hydroxyindole (5.50 g, 41.3 mmol, 1 equiv), tert-butyldimethylsilyl chloride (7.47 g, 49.6 mmol, 1.20 equiv), and imidazole (7.03 g, 103 mmol, 2.50 equiv) in N,N-dimethylformamide (20 mL) was stirred at 23° C. for 20 h. The reaction mixture was concentrated and the residue was partitioned between ethyl acetate and water. The organic layer was washed with water (3×), then dried over magnesium sulfate and concentrated. The residue was purified by flash column chromatography (40% dichloromethane in hexanes, then 60% dichloromethane in hexanes) to give 5-(tert-butyl-dimethyl-silanyloxy)-1H-indole (intermediate B) as a colorless oil which solidified upon standing. ¹H NMR (400 MHz, CDCl₃) δ8.00 (br s, 1H), 7.22 (d, 1H, J=8.7 Hz), 7.17 (t, 1H, J=2.8 Hz), 7.06 (d, 1H, J=2.3 Hz), 6.76 (dd, 1H, J=8.6, 2.3 Hz), 6.44 (m, 1H), 1.00 (s, 9H), 0.19 (s, 6H).

Step C: Synthesis of 5-(tert-butyl-dimethyl-silanyloxy)-indole-1-carboxylic acid tert-butyl ester (Intermediate C)

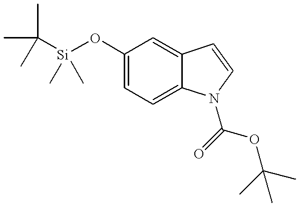

A solution of intermediate B (10.2 g, 41.3 mmol, 1 equiv), di-tert-butyl dicarbonate (14.4 g, 66.0 equiv, 1.60 equiv), and 4-dimethylaminopyridine (1.01 g, 8.25 mmol, 0.200 equiv) in dichloromethane (100 mL) was stirred at 23° C. for 20 h. The reaction mixture was concentrated, and the residue was purified by flash column chromatography (40% dichloromethane in hexanes) to afford 5-(tert-butyl-dimethyl-silanyloxy)-indole-1-carboxylic acid tert-butyl ester (intermediate C) as a colorless oil. ¹H NMR (400 MHz, CDCl₃) δ7.96 (br d, 1H, J=7.5 Hz), 7.54 (br d, 1H, J=3.1 Hz), 6.98 (d, 1H, J=2.4 Hz), 6.83 (dd, 1H, J=9.0, 2.4 Hz), 6.45 (d, 1H, J=3.7 Hz), 1.66 (s, 9H), 1.00 (s, 9H), 0.20 (s, 6H).

Step D: Synthesis of Intermediate D

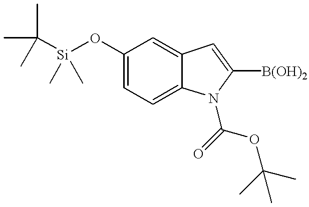

A solution of tert-butyllithium in pentane (1.7 M, 20.7 mL, 35.2 mmol, 1.20 equiv) was added to a solution of intermediate C (10.2 g, 29.3 mmol, 1 equiv) in tetrahydrofuran (100 mL) at −78 deg C. The resulting light-brown solution was stirred at −78° C. for 30 min. Trimethylborate (6.67 mL, 58.7 mmol, 2.00 equiv) was then added. The resulting mixture was warmed to 0° C. and then diluted with saturated aqueous ammonium chloride solution (100 mL) and ethyl ether (200 mL). The aqueous layer was made acidic with aqueous 10% potassium hydrogensulfate solution. The organic layer was separated, washed with brine, dried over magnesium sulfate, and concentrated. The residual yellow solid was triturated with hexanes to give intermediate D as an off-white solid. ¹H NMR (400 MHz, CDCl₃) δ7.84 (d, 1H, J=8.9 Hz), 7.37 (s, 1H), 7.01 (d, 1H, J =2.4 Hz), 6.97 (br s, 2H), 6.88 (dd, 1H, J=9.0, 2.4 Hz), 1.73 (s, 9H), 1.00 (s, 9H), 0.20 (s, 6H).

Step E: Synthesis of Intermediate E

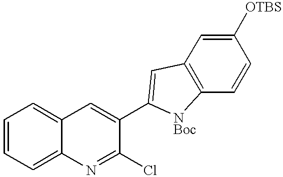

A deoxygenated mixture of intermediate D (4.10 g, 10.5 mmol, 1 equiv), intermediate A (3.64 g, 12.6 mmol, 1.20 equiv), potassium phosphate (6.67 g, 31.4 mmol, 3.00 equiv), and tetrakis(triphenylphosphine)palladium (0.605 g, 0.524 mmol, 0.050 equiv) in dioxane 100 mL) was heated at 90° C. for 20 h. The reaction mixture was cooled, then partitioned between a mixture of water and ethyl acetate. The organic layer was separated, washed with brine, dried over magnesium sulfate, and concentrated. The residue was purified by flash column chromtography (20% dichloromethane in hexanes, grading to 90% dichloromethane in hexanes) to give intermediate E as a tan-colored foam. ¹H NMR (400 MHz, CDCl₃) δ8.16 (s, 1H), 8.15 (d, 1H, J=9.0 Hz), 8.07 (d, 1H, J =8.2 Hz), 7.86 (d, 1H, J=7.8 Hz), 7.77 (br t, 1H, J=8.4 Hz), 7.60 (br t, 1H, J=8.1 Hz), 7.03 (d, 1H, J=2.4 Hz), 6.92 (dd, 1H, J=9.0, 2.4 Hz), 6.55 (s, 1H), 1.26 (s, 9H), 1.02 (s, 9H), 0.23 (s, 6H).

Step F: Synthesis of Intermediate F

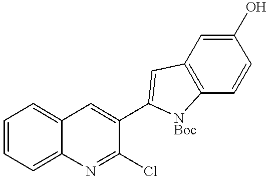

A solution of intermediate E (2.50 g, 4.91 mmol, 1 equiv) and triethylamine trihydrofluoride (3.60 mL, 22.1 mmol, 4.50 equiv) in acetonitrile (100 mL) was stirred at 23° C. for 20 h. The reaction mixture was concentrated, and the residue was partitioned between saturated aqueous sodium bicarbonate solution and ethyl acetate. The organic layer was washed with brine, dried over magnesium sulfate and concentrated to afford intermediate F as a tan colored foam. ¹H NMR (400 MHz, CDCl₃) δ8.18 (d, 1H, J=9.0 Hz), 8.17 (s, 1H), 8.07 (d, 1H, J=8.4 Hz), 7.86 (d, 1H, J=8.1 Hz), 7.77 (br t, 1H, J=8.4 Hz), 7.61 (br t, 1H, J=8.1 Hz), 7.03 (d, 1H, J=2.6 Hz), 6.93 (dd, 1H, J=8.8, 2.6 Hz), 6.55 (s, 1H), 1.26 (s, 9H).

Step G: Synthesis of Title Compound: 3-[5-(2-piperidin-1-yl-ethoxy)-1H-indol-2-yl]-1H-quinolin-2-one (10)

A mixture of intermediate F (395 mg, 1.00 mmol, 1 equiv), 1-(2-chloroethyl)-piperidine hydrochloride (276 mg, 1.50 mmol, 1.50 equiv), and cesium carbonate (978 mg, 3.00 mmol, 3.00 equiv) in N, N-dimethylformamide (5 mL) was heated at 50° C. for 2 h. The reaction mixture was concentrated, and the residue was partitioned between water and ethyl acetate. The organic layer was washed with water, then brine, dried over magnesium sulfate, and concentrated to give a pale-yellow foam. The foam was dissolved in a 1:1 mixture of water and acetic acid (60 mL), and the resulting solution was heated at 110° C. for 12 h. The reaction mixture was concentrated, and the residue was stirred in aqueous saturated sodium bicarbonate solution which yielded a tan solid. The tan solid was filtered, then suspended in warm ethanol (2×20 mL) and filtered to give compound the title product (10) as a yellow solid. The ethanolic filtrate was concentrated and the residue purified by flash column chromatography (5% ethanol saturated with ammonia in ethyl acetate to afford additional product. ¹H NMR (400 MHz, (CD₃)₂SO) δ12.14 (s, 1H), 11.41 (s, 1H), 8.50 (s, 1H), 7.73 (br d, 1H, J=7.9 Hz), 7.51 (br t, 1H, J=7.6 Hz), 7.41 (d, 1H, J=8.6 Hz), 7.37 (br d, 1H, J=8.2 Hz), 7.24 (br t, 1H, J=7.7 Hz), 7.21 (br s, 1H), 7.06 (br s, 1H), 6.76 (dd, 1H, J=8.6, 2.2 Hz), 4.06 (t, 2H, J=5.9 Hz), 2.67 (t, 3H, J=5.5 Hz), 2.45 (br m, 4H), 1.51 (br m, 4H), 1.39 (br m, 2H).

### Example 7

### 3-[5-(2-pyrrolidin-1-yl-ethoxy)-1H-indol-2-yl]-1H-quinolin-2-one

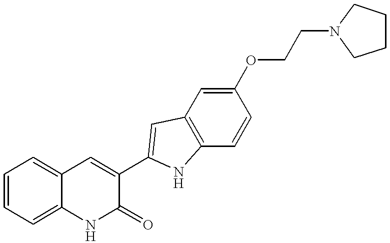

A mixture of intermediate F from Example 6 above (79 mg, 0.20 mmol, 1 equiv), 1-(2-chloroethyl)-pyrrolidine hydrochloride (51 mg, 0.30 mmol, 1.5 equiv), and cesium carbonate (196 mg, 0.60 mmol, 3.00 equiv) in N, N-dimethylformamide (1 mL) was heated at 50° C. for 3 h. The reaction mixture was concentrated, and the residue was partitioned between water (2 mL) and dichloromethane (2×2 mL). The organic layer was dried over magnesium sulfate and concentrated to give a pale-yellow oil. The oil was dissolved in a 1:1 mixture of acetic acid and water (2 mL), and the resulting solution was heated at 100° C. for 20 h. The reaction mixture was concentrated, and the residue was suspended in aqueous saturated sodium bicarbonate solution. The resulting solid was filtered, washed with water (2×2 mL) and vacuum dried. The solid was then triturated with ethanol (2×) and ethyl ether (2×), then vacuum dried. The solid was further purified by flash column chromatography (dichloromethane, grading to 7% ethanol saturated with ammonia in dichloromethane) to give the title compound as a yellow solid. ¹H NMR (400 MHz, (CD₃)₂SO) δ12.14 (s, 1H), 11.41 (s, 1H), 8.50 (s, 1H), 7.73 (br d, 1H, J=7.7 Hz), 7.51 (br t, 1H, J=7.2 Hz), 7.41 (d, 1H, J=8.6 Hz), 7.37 (br d, 1H, J=8.2 Hz), 7.24 (br t, 1H, J=7.7 Hz), 7.21 (d, 1H, J=1.3 Hz), 7.06 (d, 1H, J=2.2 Hz), 6.76 (dd, 1H, J=8.6, 2.2 Hz), 4.07 (t, 2H, J=5.9 Hz), 2.81 (t, 3H, J=5.9 Hz), 2.55 (br m, 4H), 1.70 (br m, 4H).

Examples 8-9 below were prepared by simple modifications of the protocols described abovein Examples 6 and 7:

### Example 8

### 3-(5-{2-[bis-(2-methoxy-ethyl)-amino]-ethoxy}-1h-indol-2-yl)-1h-quinolin-2-one

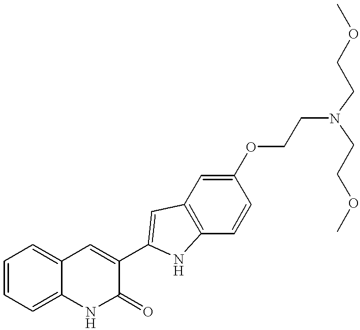

### Example 9

### 3-(5-{2-[ethyl-(2-methoxy-ethyl)-amino]-ethoxy}-1h-indol-2-yl)-1h-quinolin-2-one

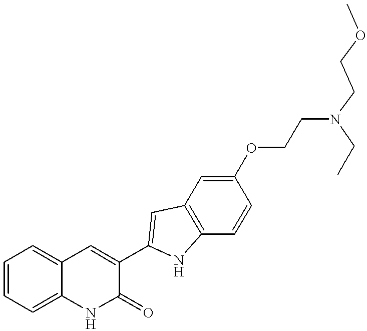

### Example 10

### 3-(5-methoxy-1H-pyrrolo[3,2-b]pyridin-2-yl)-1H-quinolin-2-one

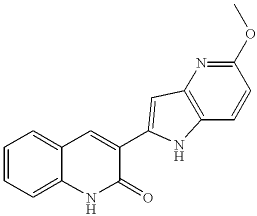

Step 1: Synthesis of 2-chloro-3-iodo-quinoline (Intermediate 10-A)

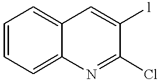

A suspension of 3-(2-chloro)-quinolineboronic acid (5.05 g, 24.3 mmol, 1 equiv, prepared by the method of Marsais, F; Godard, A.; Queguiner, G. J. Heterocyclic Chem. 1989, 26, 1589-1594) and N-iodosuccinimide (5.48 g, 24.4 mmol, 1.00 equiv) in acetonitrile (300 mL) was stirred at 23° C. in the dark for 20 h. The reaction mixture was concentrated to dryness, and the resulting yellow solid was partitioned between saturated aqueous sodium bicarbonate solution and dichioromethane. The organic layer was washed with water, then dried over magnesium sulfate and concentrated to give 2-chloro-3-iodo-quinoline (intermediate 10-A) as a pale yellow solid. ¹H NMR (400 MHz, CDCl₃) δ8.67 (s, 1H), 7.99 (br d, 1H, J=8.4 Hz), 7.75 (br t, 1H, J=7.7 Hz), 7.72 (br d, 1H, J=7.8 Hz), 7.57 (br t, 1H, J=7.6 Hz).

Step 2: Synthesis of Intermediate 10-B

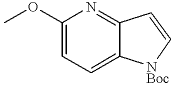

### Intermediate 10-B

A solution of 5-methoxy-1H-pyrrolo[3,2-b]pyridine (0.930 g, 6.28 mmol, 1 equiv, prepared by the method of Mazeas, D.; Guillaumet, G.; Viaud, M-C Heterocycles 1999, 50, 1065-1080), di-tert-butyl dicarbonate (1.64 g, 4.05 mmol, 1.20 equiv), and 4-dimethylaminopyridine (10 mg, 0.082 mmol, 0.013 equiv) in dichloromethane (30 mL) was stirred at 23° C. for 1 h. The reaction mixture was concentrated, and the residue was purified by flash column chromatography (100% hexanes initially, grading to 30% ethyl acetate in hexanes) to afford intermediate 10-B as a colorless oil. ¹H NMR (300 MHz, CDCl₃) δ8.24 (br d, 1H, J=9.0 Hz), 7.72 (br d, 1H, J=3.4 Hz), 6.69 (d, 1H, J=9.0 Hz), 6.63 (d, 1H, J=3.9 Hz), 3.99 (s, 3H), 1.67 (s, 9H).

Step 3: Synthesis of Intermediate 10-C

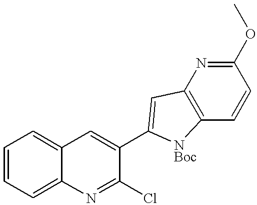

### Intermediate 10-C

Substep 1: A solution of tert-butyllithium in pentane (1.7 M, 3.95 mL, 6.72 mmol, 1.20 equiv) was added to a solution of intermediate 10-B (1.39 g, 5.60 mmol, 1 equiv) in THF (70 mL) at −78° C. The orange solution was stirred for 15 min, then a solution of trimethyltin chloride (2.23 g, 11.2 mmol, 2.00 equiv) in THF (4.0 mL) was added. The reaction mixture was warmed to 23° C., then partitioned between aqueous pH 7 phosphate buffer and a 1:1 mixture of ethyl acetate and hexane (100 mL). The organic layer was dried over sodium sulfate and concentrated.

Substep 2: A deoxygenated solution of this residue, intermediate 10-A (0.800 g, 2.76 mmol, 0.500 equiv), tetrakis(triphenylphosphine)palladium (0.160 g, 0.140 mmol, 0.025 equiv), and cuprous iodide (0.053 g, 0.28 mmol, 0.05 equiv) in dioxane (40 mL) was heated at 90 deg C. for 20 h. The reaction mixture was cooled, then partitioned between brine (150 mL) and ethyl acetate (150 mL). The organic layer was dried over sodium sulfate, then concentrated. The residue was purified by flash column chromatography (100% hexanes initially, grading to 30% ethyl acetate in hexanes) to afford intermediate 10-C as a light yellow foam. ¹H NMR (300 MHz, CDCl₃) δ8.44 (d, 1H, J=9.2 Hz), 8.18 (s, 1H), 8.08 (d, 1H, J=8.5 Hz), 7.88 (d, 1H, J=8.2 Hz), 7.79 (ddd, 1H, J=8.5, 7.0, 1.5 Hz), 7.63 (ddd, 1H, J=8.5, 7.0, 1.5 Hz), 6.78 (d, 1H, J=8.8 Hz), 6.72 (s, 1H), 4.02 (s, 3H), 1.27 (s, 9H).

Step 4: Synthesis of 3-(5-methoxy-1H-pyrrolo[3,2-b]pyridin-2-yl)-1H-quinolin-2-one

A solution of intermediate 10-C (900 mg, 2.20 mmol) was heated in a 1:1 mixture of acetic acid and water (50 mL) at reflux for 16 h. The reaction mixture was concentrated, and the residue was partitioned between aqueous saturated sodium bicarbonate solution (150 mL) and hot ethyl acetate (3×200 mL). The combined organic layers were dried over sodium sulfate and concentrated. The residue was suspended in ethyl ether (200 mL), filtered, then air-dried to give the titled compound as a yellow solid. ¹H NMR (300 MHz, (CD₃)₂SO) δ12.23 (s, 1H), 11.75 (s, 1H), 8.58 (s, 1H), 7.86 (br d, 1H, J=9.2 Hz), 7.75 (br d, 1H, J=7.6, Hz), 7.54 (br t, 1H, J=7.8 Hz), 7.39 (d, 1H, J=8.2 Hz), 7.26 (br t, 1H, J=7.6 Hz), 7.18 (br s, 1H), 6.57 (d, 1H, J=8.5 Hz), 3.88 (s, 3H). HRMS (electrospray FT/ICR) calcd for C₁₇H₁₄N₃O₂[M+H]⁺292.1081, found 292.1059.

### Example 11

### Preparation of[N-Ac-(4-trans-L-Hyp)]-Ala-Ser-Chg-Gln-Ser-Leu-Dox (SEQ.ID.NO.: 22)

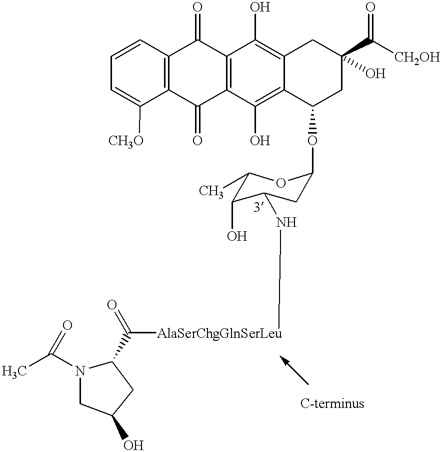

Step A: [N-Ac-(4-trans-L-Hyp(Bzl))]-Ala-Ser(Bzl)Chg-Gln-Ser(Bzl)Leu-PAM Resin (11-1).

Starting with 0.5 mmol (0.67g) Boc-Leu-PAM resin, the protected peptide was synthesized on a 430A ABI peptide synthesizer. The protocol used a 4 fold excess (2 mmol) of each of the following protected amino acids: Boc-Ser(Bzl), Boc-Gln, Boc-Chg, Boc-Ala, N-Boc-(4-trans-L-Hyp(Bzl)). Coupling was achieved using DCC and HOBT activation in methyl-2-pyrrolidinone. Acetic acid was used for the introduction of the N terminal acetyl group. Removal of the Boc group was performed using 50% TFA in methylene chloride and the TFA salt neutralized with diisopropylethylamine. At the completion of the synthesis the peptide resin was dried to yield Intermediate 11-1.

Step B: [N-Ac-(4-trans-L-Hyp)]-Ala-Ser-Chg-Gln-Ser-Leu-OH (11-2)

The protected peptide resin (11-1), 1.2 g, was treated with HF (20 ml) for 1 hr at 0° C. in the presence of anisole (2 ml). After evaporation of the HF, the residue was washed with ether, filtered and extracted with H₂O (200 ml). The filtrate was lyophilyzed to yield Intermediate 11-2.

Step C: [N-Ac-(4-trans-L-Hyp)-Ala-Ser-Chg-Gln-Ser-Leu-Dox

The above described intermediate (11-2), 1.157 g (1.45 mmol) was dissolved in DMSO (30 ml) and diluted with DMF (30 ml). To the solution was added doxorubicin hydrochloride, 516 mg (0.89 mmol) followed by 0.310 mL of diisopropylethylamine (1.78 mmol). The stirred solution was cooled (0° C.) and 0.276 mL of diphenylphosphoryl azide (1.28 mmol) added. After 30 minutes, an additional 0.276 mL (1.28 mmol) of DPPA was added and the pH adjusted to ˜7.5 (pH paper) with diisopropylethylamine (DIEA). The pH of the cooled reaction (0° C.) was maintained at ˜7.5 with DIEA for the next 3 hrs. and the reaction stirred at 0-4° C. overnight. After 18 hrs., the reaction (found to be complete by analytical HPLC, system A) was concentrated to an oil. Purification of the crude product was achieved by preparative HPLC, Buffer A=0.1% NH₄OAc-H₂O; B=CH₃CN. The crude product was dissolved in 400 mL of 100% A buffer, filtered and purified on a C-18 reverse phase HPLC radial compression column (Waters, Delta-Pak, 15 μM, 100 Å). A step gradient of 100% A to 60% A was used at a flow rate of 75 ml/min (UV=214 nm). Homogeneous product fractions (evaluated by HPLC, system A) were pooled and freeze-dried. The product was dissolved in H₂O (300 ml), filtered and freeze-dried to provide the purified title compound.

PHYSICAL PROPERTIES
The physical/chemical properties of the product of Step C
are shown below:

| | |
|---|---|
| Molecular Formula: | C₆₂H₈₅N₉O₂₃ |
| Molecular Weight: | 1323.6 |
| High Resolution ES Mass Spec: | 1341.7 (NH₄⁺) |
| HPLC: | System A |
| Column: | Vydac 15 cm #218TP5415, C18 |
| Eluant: | Gradient 95:5 (A:B) to 5:95 |
| | (A:B) over 45 min. A = 0.1% |
| | TFA/H₂O, B = 0.1% |
| | TFA/Acetonitrile |
| Flow: | 1.5 ml/min. |
| Wavelength: | 214 nm, 254 nm |
| Retention Time: | 18.2 mm. |

Amino Acid Compositional Analysis¹:

| Theory | Found |
|---|---|
| Ala (1) | 1.00 |
| Ser (2) | 1.88 |
| Chg (1) | 0.91 |
| Gln² (1) | 1.00 (as Glu) |
| Hyp (1) | 0.80 |
| Leu (1) | 1.01 |
| Peptide Content: | 0.657 μmol/mg |

Note:
¹20 hr., 100° C., 6N HCl
²Gln converted to Glu

### Example 12

### Preparation of[N-Glutaryl-(4-trans-L-Hyp)]-Ala-Ser-Chg-Gln-Ser-Leu-Dox (SEQ.ID.NO.:25) (Compound 11)

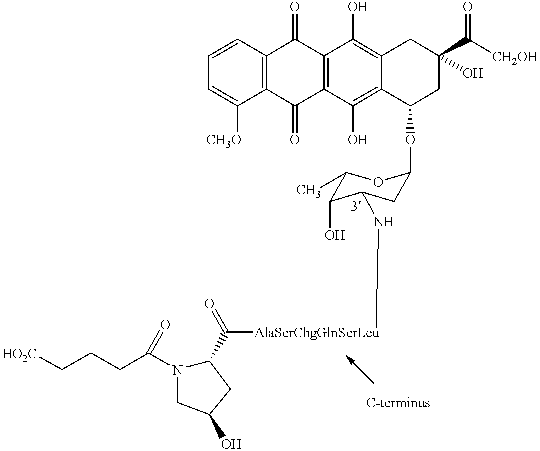

Step A: [N-Glutaryl(OFm)-(4-trans-L-Hyp)]-Ala-Ser-Chg-Gln-Ser-Leu-PAM Resin

Starting with 0.5mmol (0.67 g) Boc-Leu-PAM resin, the protected peptide was synthesized on a 430A ABI peptide synthesizer. The protocol used a 4 fold excess (2 mmol) of each of the following protected amino acids: Fmoc-Ser(tBu), Fmoc-Gln(Trt), Fmoc-Chg, Fmoc-Ala, Boc-(4-trans-L-Hyp). Coupling was achieved using DCC and HOBT activation in methyl-2-pyrrolidinone. The intermediate mono fluorenylmethyl ester of glutaric acid [Glutaryl(OFm)] was used for the introduction of the N-terminal glutaryl group. Removal of the Fmoc group was performed using 20% piperidine. The acid sensitive protecting groups, Boc, Trt and tBu, were removed with 50% TFA in methylene chloride. Neutralization of the TFA salt was with diisopropylethylamine. At the completion of the synthesis, the peptide resin was dried to yield the title compound.

Step B: [N-Glutaryl(OFm)-(4-trans-L-Hyp)]-Ala-Ser-Chg-Gln-Ser-Leu-OH

The protected peptide resin from Step A, 1.2 g, was treated with HF (20 ml) for 1 hr at 0° C. in the presence of anisole (2 ml). After evaporation of the HF, the residue was washed with ether, filtered and extracted with DMF. The DMF filtrate (75 ml) was concentrated to dryness and triturated with H₂O. The insoluble product was filtered and dried to provide the title compound.

Step C: [N-Glutaryl(OFm)-(4-trans-L-Hyp)] -Ala-Ser-Chg-Gln-Ser-Leu-Dox

The above prepared intermediate from Step B, (1.33 g, 1.27mmol) was dissolved in DMSO (6 ml) and DMF (69 ml). To the solution was added doxorubicin hydrochloride, 599 mg (1.03 mmol) followed by 376 μl of diisopropylethylamine (2.16 mmol). The stirred solution was cooled (0° C.) and 324 μl of diphenylphosphoryl azide (1.5 mmol) added. After 30 minutes, an additional 324 μl of DPPA was added and the pH adjusted to ˜7.5 (pH paper) with diisopropylethyl-amine (DIEA). The pH of the cooled reaction (0° C.) was maintained at ˜7.5 with DIEA for the next 3 hrs and the reaction stirred at 0-4° C. overnight. After 18 hrs., the reaction (found to be complete by analytical HPLC, system A) was concentrated to provide the title compound as an oil.

Step D: [N-Glutaryl-(4-trans-L-Hyp)]-Ala-Ser-Chg-Gln-Ser-Leu-Dox

The above product from Step C was dissolved in DMF (54 ml), cooled (0° C.) and 14 mL of piperidine added. The solution was concentrated to dryness and purified by preparative HPLC. (A=0.1% NH4OAc-H₂O; B=CH₃CN.) The crude product was dissolved in 100 mL of 80% A buffer, filtered and purified on a C-18 reverse phase HPLC radial compression column (Waters, Delta-Pak, 15μ, 100 Å). A step gradient of 80% A to 67% A was used at a flow rate of 75 ml/min (uv=214 nm). Homogeneous product fractions (evaluated by HPLC, system A) were pooled and freeze-dried. The product was further purified using the above HPLC column. Buffer A=15% acetic acid-H₂O; B=15% acetic acid-methanol. The product was dissolved in 100 mL of 20% B/80% A buffer and purified. A step gradient of 20% B to 80% B was used at a flow rate of 75 ml/min (uv=260 nm). Homogeneous product fractions (evaluated by HPLC, system A) were pooled, concentrated and freeze-dried from H₂O to yield the purified title compound.

| | |
|---|---|
| High Resolution ES Mass Spec: | 1418.78 (Na⁺) |
| HPLC: | System A |
| Column: | Vydac 15 cm #218TP5415, C18 |
| Eluant: | Gradient 95:5 (A:B) to 5:95 |
| | (A:B) over 45 min. A = 0.1% |
| | TFA/H₂O, B = 0.1% |
| | TFA/Acetonitrile |
| Flow: | 1.5 ml/min. |
| Wavelength: | 214 nm, 254 nm |
| Retention Time: | 18.3 min. |

Amino Acid Compositional Analysis¹:

| Theory | Found |
|---|---|
| Ala (1) | 0.99 |
| Ser (2) | 2.02 |
| Chg (1) | 1.00 |
| Gln² (1) | 1.01 (as Glu) |
| Hyp (1) | 0.99 |
| Leu (1) | 1.00 |
| Peptide Content: | 0.682 μmol/mg |

Note:
¹20 hr., 100° C., 6N HCl
²Gln converted to Glu

### EXAMPLE 12A

### Preparation of [N-Glutaryl-(4-trans-L-Hyp)]-Ala-Ser-Chg-Gln-Ser-Leu-Dox Sodium Salt (SEQ.ID.NO.:25)

### Preparation N-(N′-(Fm-Glutaryl)-trans-4-hydroxy-L-prolinyl-alanyl)serine

Step 1: N-Boc-trans-4-hydroxy-L-proline

A solution of trans-4-hydroxy-L-proline (3.0 kg, 22.88 M) in 1 M aqueous sodium hydroxide (25.2 L) and tert-butanol (12.0 L) was treated with a solution of di-tert-butyldicarbonate (5.09 kg) in tert-butanol (6.0 L) at 20° C. over 20 minutes. Upon complete addition, the resulting solution was stirred at 20° C. for 2 hours. The solution was extracted with hexane (2×15.0 L) and then acidified to pH 1 to 1.5 by cautious addition of a solution of potassium hydrogen sulphate (3.6 kg) in water (15.0 L). The mixture was extracted with ethyl acetate (3×15.0 L). The combined ethyl acetate extracts were washed with water (2×1.0 L) and dried by azeotropic distillation at atmospheric pressure (final KF of ethyl acetate solution<0.1%).

The ethyl acetate solution was then concentrated by atmospheric distillation to a volume of 15.0 L, diluted with hexane (8.0 L), seeded and stirred at 20° C. for 1 hour. Hexane (22.5 L) was added over 2 hours, the slurry was cooled to 0° C. for 1 hour and the solid collected by filtration. The product was washed with cold (0° C.) 2:1 hexane/ethyl acetate (15.0 L) and dried in vacuo at 45° C. to afford the title compound as a white crystalline solid.

Step 2: N-Boc-trans-4-hydroxy-L-proline Pentafluorophenyl ester

Boc-trans-4-hydroxy-L-proline (3.5 kg) (prepared as described in Step 1) and pentafluorophenol (3.06 kg) were dissolved in ethyl acetate (52 L). The solution was treated with a solution of dicyclohexylcarbodiimide (3.43 kg) in ethyl acetate (8 L) and the mixture was stirred at room temperature for 2 hours. The resulting slurry was cooled to 0° C., filtered and the solids washed with ethyl acetate (15 L). The filtrate was evaporated at atmospheric pressure to a volume of 10 L and diluted with hexane (100 L). The resulting mixture was stirred at room temperature overnight and then cooled to 0° C. for 1 hour. The solid was collected by filtration, washed with cold (° C.) 10:1 hexane/ethyl acetate (15 L) and dried at 45° C. in vacuo to afford the title compound as a white crystalline solid.

Step 3: N-(trans-4-hydroxy-L-prolinyl-alanyl)serine hydrochloride

N-alanylserine (1.5 kg, 8.515 M) and Boc-trans-4-hydroxy-L-proline (3.72 kg) (prepared as described in step 2) were heated at 50° C. in dimethylformamide (15 L) for 3 hours. The solution was cooled to 20° C., treated with concentrated hydrochloric acid (7.5 L) and stirred at room temperature for 24 hours. The resulting slurry was diluted with isopropanol (30 L), stirred at room temperature for 30 minutes and then cooled to 0° C. for 1 hour. The solid was collected by filtration and washed with isopropanol (20 L). The solid was dried in vacuo at 40° C. to afford the title compound as a white crystalline solid.

Step 4: Fluorenylmethyl Glutarate

9-Fluorenyl methanol (2.0 kg), glutaric anhydride (2.33 kg) and sodium bicarbonate (1.71 kg) were stirred together in N-methylpyrrolidinone (8.0 L) at room temperature for 72 hours. The slurry was filtered and the solids washed with isopropyl acetate (2×10.0 L). The filtrate was washed with 1.0 M hydrochloric acid (3×10.0 L). The organic layer was extracted with 1.0 M aqueous sodium hydroxide (3×8.0 L). The combined basic extracts were covered with isopropyl acetate (20.0 L) and acidified to pH 2 with 2.0 M hydrochloric acid (12.5 L). The phases were separated and the aqueous phase was extracted with isopropyl acetate (10.0 L).

The combined organic phases were washed with water (10.0 L) and dried by azeotropic distillation at <60° C. under reduced pressure (KF<0.05%). The solution was then concentrated under reduced pressure (<60° C.) to a volume of 7.0 L. The solution was diluted with hexane (6.0 L), seeded and stirred at room temperature for 30 minutes. The resulting slurry was diluted by addition of hexane (42.0 L) over 40 minutes. The slurry was cooled to 0° C. for 1 hour and the solid collected by filtration and washed with cold (0° C.) 8:1 hexane/iPAc (20.0 L). The solid was dried in vacuo at 45° C. to afford the title compound as a pale cream solid.

Step 5: Fluorenylmethyl Glutarate Pentafluorophenyl Ester

Fluorenylmethyl glutarate (2.5 kg) (prepared as described in Step 4) and pentafluorophenol (1.63 kg) were dissolved in ethyl acetate (25 L). The solution was treated with a solution of dicyclohexylcarbodiimide (1.83 kg) in ethyl acetate (7.5 L) and the mixture was stirred at 20° C. overnight. The resulting slurry was filtered and the solids were washed through with ethyl acetate (10 L). The filtrate was evaporated at atmospheric pressure to a volume of 7.5 L and diluted with hexane (75 L). The slurry was filtered at 60-65° C. then allowed to cool to room temperature and stirred overnight. The slurry was cooled to 0° C. for 1 hour, the solid collected by filtration and washed with 10:1 hexane/ethyl acetate (15 L). The solid was dried in vacuo at 45° C. to afford the title compound as a white crystalline solid.

Step 6: N-(N′-(Fm-Glutaryl)-trans-4-hydroxy-L-prolinyl-alanyl)serine

N-(trans-4-hydroxy-L-prolinyl-alanyl)serine hydrochloride (2.3 kg) (prepared as described in Step 3) was suspended in dimethylformamide (22 L) and the slurry was treated with N-ethylmorpholine (911 ml) followed by a solution of fluorenylmethyl glutarate pentafluorophenyl ester (3.5 kg) (prepared as described in Step 5) in dimethylformamide (14 L). The mixture was heated at 50° C. for 3 hours and the resulting solution evaporated to residue under reduced pressure. The residue was partitioned between water (80 L) and tert-butyl methyl ether (34 L). The phases were separated and the aqueous layer was extracted with tert-butyl methyl ether (34 L). The aqueous solution was seeded and stirred at room temperature overnight. The solid was collected by filtration (slow) and washed with water (25 L). The damp filter cake was dissolved in isopropanol (90 L) with warming and the solution concentrated to half volume by distillation at atmospheric pressure. Additional portions of isopropanol (3×45 L) were added and the batch was concentrated to ca half volume by atmospheric distillation after addition of each portion (Final KF of liquors<0.5%). The slurry was diluted with isopropanol (23 L), stirred at 20° C. overnight, cooled to 0° C. for 1 hour and the solid collected by filtration. The cake was washed with isopropanol (20 L) and the solid dried in vacuo at 45° C. to afford the crude product as a white solid.

Step 7: Recrystallisation of N-(N′(Fm-Glutaryl)-trans-4-hydroxy-L-prolinyl- alanyl)serine

N-(N′-(Fm-Glutaryl)-trans-4-hydroxy-L-prolinyl-alanyl)serine (3.4 kg) (prepared as described in Step 6) was dissolved in methanol (51 L) at reflux. The solution was filtered and concentrated by atmospheric distillation to a volume of 17 L (5 ml/g). The solution was diluted with ethyl acetate (102 L) allowed to cool to 20° C. and stirred overnight. The resulting slurry was cooled to 0° C. for 1 hour and the solid was collected by filtration. The cake was washed with cold (0° C.) 10:1 ethyl acetate/methanol (20 L) and dried in vacuo at 45° C. to afford the product as a white solid.

### Preparation N-(cyclohexylglycyl-glutaminyl-serinyl)leucine benzyl ester hydrochloride (SEQ.ID.NO.: 47)

Step 8: N-(serinyl)leucine benzyl ester hydrochloride

Leucine benzyl ester p-tosylate (1000 g) and HOBt (412 g) were slurried in isopropyl acetate (12 L). The mixture was cooled to 0° C. in an ice-bath and a slurry of sodium bicarbonate (469.7 g) in water (1 L), N-BOC-L-serine (573.6 g) in water (2 L) and EDC.HCl (560.2 g) in water (2L) were added. The mixture was allowed to warm to 20° C. over 30 minutes and aged at 20° C. for 2 hours (<1 A % Leu-OBn remaining). If the reaction was not complete after 2 hours, further NaHCO₃ and EDC.HCl were added. The phases were separated and the organic layer was washed sequentially with saturated sodium bicarbonate (2×3.75 L), 0.5 M sodium hydrogen sulphate (2×3.75 L) and water (2×2.5 L).

The wet, isopropyl acetate solution was concentrated under reduced pressure to 3 L and the water content checked. (KF=0.12%. It is important that this solution is dry prior to the addition of hydrogen chloride in isopropyl acetate). The solution was transferred to a 20 L round bottom flask under a nitrogen atmosphere and cooled to 0° C. To the solution was added 3.6 M HCl in isopropyl acetate (7 L, 10 mol equiv. HCl). The product began to crystallize after 5 minutes. The reaction was aged at 0° C. for 1 hr, and then allowed to warm to room temperature.

The slurry was cooled to 0-5° C., diluted with heptane (2.5 L) and aged at 0° C. for 30 minutes. The product was collected by filtration, washed with cold isopropyl acetate/heptane (4:1) (2.5 L) and dried in vacuo at 35° C., with a nitrogen sweep.

Step 9: N-(N′-(Boc)-glutaminyl-serinyl)leucine benzyl ester

N-(serinyl)leucine benzyl ester hydrochloride (350 g) (prepared as described in Step 8), HOBt (157.7 g) and N-Boc-L-glutamine (262.5 g) were slurried in DMF (2.5 L) and the mixture was cooled to 0° C. N-Ethylmorpholine (245.5 g) and EDC.HCl (214 g) were added and the mixture was aged at 0° C. for 2.5 hours. Water (14.7 L) was added over 20 minutes and the white slurry aged at 0° C. for 1 hour. The product collected by filtration and washed with water (3.2 L). The cake was dried in the fume-hood overnight. The isolated N-BOC-Gln-Ser-Leu-OBn, which contained DMF and HOBt, was combined with a second batch of identical size, and swished in water (12 L) at 20° C. for 1 hour. The product was collected by filtration, washed with water (2.5 L) and air-dried in a fume-hood over the weekend. The batch was dried in vacuo, at 42° C., with a nitrogen bleed.

Step 10: N-(glutaminyl-serinyl)leucine benzyl ester hydrochloride

N-(N′-(Boc)-glutaminyl-serinyl)leucine benzyl ester (715 g, 1.33 M) (prepared as described in Step 9) was suspended in iPAc (3.5 L) at room temperature. To the slurry was added a 3.8 M solution of HCl in iPAc (3.5 L, 13.3 M) whereupon all the solids dissolved. After a short time, the product crystallized. The mixture was stirred at room temperature for 3.75 hours when HPLC showed complete reaction. The slurry was diluted with iPAc (4.0 L), stirred for 1 hour at room temperature and the solid collected by filtration under nitrogen. The product is very hygroscopic in the presence of excess HCl and must be collected under dry nitrogen.

The cake was washed with iPAc (4.0 L), the solid dried on the filter under nitrogen for 2 hours and then dried in vacuo at 45° C.

Step 11: N-(N′-(Boc)-cyclohexylglycylglutaminyl-serinyl)leucine-benzyl ester(SEQ.ID.NO.: 47)

N-(glutaminyl-serinyl)leucine benzyl ester hydrochloride (2.6 kg) (prepared as described in Step 10), N-Boc-L-cyclohexylglycine (1.414 kg) and HOBt hydrate (168 g) were dissolved in DMF (13.0 L). N-ethylmorpholine (1.266 kg, 11.0 M) and EDC hydrochloride (1.265 kg) were added and the mixture stirred at 20° C. for 3 hours. The solution was diluted with ethyl acetate (13.0 L) and water (26.0 L) added. The product precipitated and the slurry was stirred at room temperature for 1 hour. The solid was collected by filtration, washed with 1:1 ethyl acetate/water (60 L) dried on the filter under nitrogen for 24 hours and dried in vacuo at 45°. The title compound was obtained as a white solid.

Step 12: N-(cyclohexylglycyl-glutaminyl-serinyl)leucine benzyl ester hydrochloride (SEQ.ID.NO.: 47)

N-(N′-(Boc)-cyclohexylglycylglutaminyl-serinyl)leucine benzyl ester (1850 g) (prepared as described in Step 11) was slurried in isopropyl acetate (3.2 L). The slurry was cooled to 0° C. in an ice bath and 3.8 M HCl/isopropyl acetate (3.7 L, 11.4 mol equiv.) was added over 5 minutes, maintaining the temperature between 8 and 10° C. The starting material had dissolved after 15-20 minutes. The solution was seeded and the reaction aged at 8-10° C. for 2 hrs, (<1A % N-Boc-tetrapeptide-OBn remaining). The batch was filtered, under a nitrogen blanket, washed with cold (10° C.) isopropyl acetate (4×3 L) then dried on the filter under nitrogen. The solid was dried in vacuo, at 40° C.

The crude N-(cyclohexylglycyl-glutaminyl-serinyl)leucine benzyl ester hydrochloride (2.2 Kg) was slurried in methanol (22.3 L) at room temperature. The batch was stirred for 1 hour and then ethyl acetate (44.6 L) was added over 30 minutes. The batch was cooled to 0-5° C., aged for one hour, then filtered and washed with cold (0-5° C.) methanol/ethyl acetate (6 L, 1:2). The solid was dried on the filter, under nitrogen, for 45 minutes and then dried in vacuo, at 40° C., with a nitrogen sweep.

The N-(cyclohexylglycyl-glutaminyl-serinyl)leucine benzyl ester hydrochloride (1.478 Kg) was slurried in methanol (14.8 L) at room and the batch stirred for 1 hr. Ethyl acetate (29.6 L) was added over 30 minutes, the batch was cooled to 0-5° C. and aged for an hour. The solid collected by filtration, washed with cold (0-5° C.) methanol/ethyl acetate (4.5 L, 1:2), dried on the filter for 45 minutes, under nitrogen, and then dried under vacuum, at 40° C. This material was then utilized in subsequent reactions.

### Preparation N-(N′-(Fm-Glutaryl)-trans-4-hydroxy-L-prolinyl-alanyl-serine-cyclohexylglycyl-glutaminyl-serinyl)leucine (Compound 12) (SEQ.ID.NO.: 48)

Step 13: N-(N′-(Fm-Glutaryl)-trans-4-hydroxy-L-prolinyl-alanyl-serine- cyclohexylglycyl-glutaminyl-serinyl)leucine benzyl ester (SEQ.ID.NO.: 49)

N-(cyclohexylglycyl-glutaminyl-serinyl)leucine benzyl ester hydrochloride (500 g) (prepared as described above), N-(N′-(Fm-Glutaryl)-trans-4-hydroxy-L-prolinyl-alanyl)serine (490 g) (prepared as described above) and HOAt (160 g) were slurried in DMF (8.2 L) and cooled to 2° C. in an ice bath. N-ethylmorpholine (135 ml) was added followed by EDC.HCl (210 g). The mixture was stirred at 0-2° C. for 2 hours and sampled. HPLC showed 0.2 A % tetrapeptide remaining. The reaction mixture was diluted with ethyl acetate (4 L) and transferred to a 30-gallon glass vessel through a 5μin-line filter. The flask and lines were rinsed with ethyl acetate/DMF (1:1, 500 ml) and ethyl acetate (4 L). Water (16.4 L) was added over 25 minutes (temperature 11° C. to 23° C.) and the mixture stirred slowly, at 20° C., for 30 minutes. The product was collected by filtration, washed with water (3 L), ethyl acetate (1 L) and water (2×3 L), then dried on the filter under nitrogen, and dried in vacuo at 45° C.

Alternate Step 13: Fm-Glutaryl-Hyp-Ala-Ser-Chg-Gln-Ser-Leu-O-benzyl (SEQ.ID.NO.: 49)

HCl.H-Chg-Gln-Ser-Leu-OBn (100 g), Fm-Glutaryl-Hyp-Ala-Ser-OH (98 g) and 4-hydroxypyridine-N-oxide (HOPO, 18.2 g) were slurried in DMF (1.6 L) and cooled to 2° C. in an ice bath. N-ethylmorpholine (27 ml) was added followed by EDC.HCl (42 g). The mixture was stirred at 2-5° C. for 4 hours and sampled. HPLC showed 0.6 A % tetrapeptide remaining. The reaction mixture was diluted with ethyl acetate (1.64 L), water (3.3 L) was added over 70 minutes and the mixture stirred slowly, at 20° C., for 60 minutes. The product was collected by filtration, washed with water (1.5 L), ethyl acetate (1 L) and water (3×1 L), then dried on the filter under nitrogen, and dried in vacuo at 45° C.

Step 14: N-(N′-(Fm-Glutaryl)-trans-4-hydroxy-L-prolinyl-alanyl-serine-cyclohexylglycyl-glutaminyl-serinyl)leucine (SEQ.ID.NO.: 48)

N-(N′-(Fm-Glutaryl)-trans-4-hydroxy-L-prolinyl-alanyl-serine-cyclohexylglycyl-glutaminyl-serinyl)leucine benzyl ester (1.1 Kg) (prepared as described in Step 13) was dissolved in dimethylacetamide (7.8 L) containing methanesulphonic acid (93.5 ml). 5% Pd/C (110 g, 10 wt %), slurried in DMA (1.0 L), was added and the mixture hydrogenated at atmospheric pressure for 1 hour 40 minutes. The reaction mixture was sampled: HPLC showed no starting material remaining.

The reaction mixture was filtered through a pre-wetted (DMA) pad of hyflo (500 g) to remove the catalyst. The hyflo pad washed with DMA (2.2 L) and then ethyl acetate (5.5 L). The filtrate was diluted with ethyl acetate (5.5 L) and stirred for 15 minutes. Water (44 L) was added over 40 minutes and the batch age for 1 hour. The solid collected by filtration, washed with water (1×10 L, 3×20 L), dried on the filter under a nitrogen blanket and dried in vacuo at 45° C.

Step 15: N-(N′-(Fm-Glutaryl)-trans-4-hydroxy-L-prolinyl-alanyl-serine-cyclohexylglycyl-glutaminyl-serinyl)leucine Swish Purification

Crude N-(N′-(Fm-Glutaryl)-trans-4-hydroxy-L-prolinyl-alanyl-serine-cyclohexylglycyl-glutaminyl-serinyl)leucine (2.58 kg) (prepared as described in Step 14) was sieved.

The solid (2.56 Kg) was swished in ethyl acetate for 3 hours. The solid was collected by filtration, washed with ethyl acetate (26 L), dried on the filter under nitrogen and dried in vacuo at 40° C. The product was analyzed for purity by HPLC:

Step 16: Preparation of [N-Glutaryl(OFm)-(4-trans-L-Hyp)]-Ala-Ser-Chg-Gln-Ser-Leu-Dox (Compound 13) (SEQ.ID.NO.: 25)

To a 3 necked, 12 L round bottom flask equipped with mechanical stirrer, thermocouple, and nitrogen inlet was charged DMF (5.1 L) and HOAt (43.4 g, 319 mmoles, 1.2 equivalents). The yellow solution was inerted with nitrogen and warmed to 40° C. Heptapeptide prepared as described in Step 15(357.34 g, 266 mmoles) was added portion-wise to the warm solution; after stirring for 30 minutes at 40° C., a light yellow, opaque, homogeneous mixture resulted.

The mixture was cooled to room temperature, doxorubicin was added (158.9 g, 274 mmoles, 1.03 equivalents), and the red slurry was further cooled to −5° C. One equivalent of collidine (35 ml) was added followed by 0.8 equivalents of EDC (40.8 g, 213 mmoles) followed by the remaining two equivalents of collidine (70 ml). The red slurry was aged at −5° C. to −3° C.

The reaction was monitored by HPLC. After 1 hour, conversion had reached 58 A % Compound 13 and the remaining 0.5 eq. EDC (30.6 g, 160 mmoles) was charged.

After aging for a total of 3 hours, conversion had reached 90 A % Compound 13, 2.5 A % Heptapeptide and the reaction was warmed to 0° C. Aging for another 2 hours reduced peptide level to 0.73A % and the reaction was quenched as follows.

In a 50 L, 4 necked round bottom flask equipped with a mechanical stirrer, thermocouple, and nitrogen inlet, was charged K₂HPO₄ (67.9 g), KH₂PO₄ (283 g), and water (13 L) to give a 0.19 M pH 6.3 buffer solution. The buffer solution was inerted with nitrogen, cooled to 15-18° C., and the cold reaction mixture (−1° C.) was added to the buffer via an addition funnel over 60 minutes maintaining the slurry temperature at 15-18° C. After complete addition, the red slurry was aged 15 minutes at 18° C., and filtered. The filter cake was displacement washed with water (1×6 L), followed by slurry washing with water (6×6 L), and dried in vacuo at room temperature with a nitrogen sweep. After drying for 48 hours, a red solid with a TG. of 1.4% was obtained. The solid was analyzed by HPLC.

D-leucine Compound 13 Epimer assayed to 2.7 A %; the combined loss to the mother liquors and water washes was ca. 4% (long gradient assay). No residual peptide was detectable; the residual doxorubicin level was 1.1 A % (long gradient assay).

Step 16A: Alternate Preparation of [N-Glutaryl(OFm)-(4-trans-L-Hyp)]-Ala-Ser-Chg-Gln-Ser-Leu-Dox (Compound 13) (SEQ.ID.NO.: 25)

DMF (400 mL) was charged to a 1 L RB flask and degassed by N₂ sparge while cooling to −6° C. The Heptapeptide prepared as described in Step 15,(19.97 g, 19.06 mmol) and HOAT (3.12 g, 22.9 mmol) were then charged as solids to the cold DMF. A slurry of doxorubicin-HCl (11.05 g, 19.06 mmol) in degassed DMF (50 mL) was charged by vacuum, followed by two rinses (2×25 mL) of the slurry flask. Collidine was charged followed by a portion of EDC (2.92 g, 0.8 eq.). After 1.3 h, a second charge of EDC (2.19 g, 0.6 eq) was made. After a total age of 7.4 h the clear red solution was queched by dropwise addition to a pH 6.2 phosphate buffer (1350 mL) at 16-17° C. over 1.3 h. The resulting slurry was filtered and the filter cake was then washed with water (2000 mL). The filter cake was dried under a N₂ stream to provide the title compound as a red powder.

Step 17: Preparation of [N-Glutaryl-(4-trans-L-Hyp)]-Ala-Ser-Chg-Gln-Ser-Leu-Dox Piperidine salt (Compound 14) (SEQ.ID.NO.: 22)

To a 3 necked, 12 L round bottom flask equipped with mechanical stirrer, thermocouple, and nitrogen inlet was charged Compound 13 (399 g, 253.5 mmoles, TG 1.4%) and DMF (3.55 L). The red solution was inerted with nitrogen, cooled to 1° C., and a solution of piperidine (40 mL, 404 mmoles, 1.6 eq.) in DMF (400 mL) was added drop-wise over 70 minutes maintaining the batch temperature at 0-2° C. The resulting purplish solution was aged under nitrogen at 0-2° C.

The reaction was monitored by HPLC. After aging 1.5 hours at 0-2° C., conversion had reached 92.4% [A % 14/(A % 14+A % 13)]. Additional piperidine was charged after 2 hours reaction time (2.5 mL piperidine in 25 mL DMF); after aging another 2 hours, conversion had reached 98.1% and the reaction was quenched as follows.

In a 22 L, 3 necked round bottom flask equipped with mechanical stirrer, thermocouple, and nitrogen inlet was charged isopropyl acetate (12.1 L), inerted with nitrogen, and cooled to 0-5C. To the cold i-PAc was added the cold (2° C.) reaction mixture via nitrogen pressure cannulation over 40 minutes. The resulting pink slurry was aged at 0-5° C. for thirty minutes then filtered under nitrogen. The cake was displacement washed with i-PAc (2×4 L) then slurry washed with i-PAc (3×4 L). All washes were done under a nitrogen blanket. The solid was dried in vacuo at room temperature with a nitrogen sweep for 24 hours to give of an orange solid. The solid was assayed for purity using LC.

Step 18: Preparative HPLC purification of [N-Glutaryl-(4-trans-L-Hyp)]-Ala-Ser-Chg-Gln-Ser-Leu-Dox Piperidinium salt/Free Acid (Compound 15) (SEQ.ID.NO.: 25)

The crude piperidine salt was purified by preparative HPLC on C-18 silica gel, eluting with a 0.1% aqueous ammonium acetate/acetonitrile gradient (100% NH₄OAc to 55% NH₄OAc over 80 min). The rich cuts that were >97% pure were pooled to provide the purified piperidine salt.

A portion of the purified piperidine salt of Compound 15 was rechromatographed on C-18 silica gel using a 2% aqueous HOAc/acetonitrile gradient (100% aqueousHOAc to 40% aqueous HOAc over 60 min). The fractions that were >98% pure were pooled and lyophilized, providing the pure free acid 15.

Step 19: Preparation of [N-Glutaryl-(4-trans-L-Hyp)]-Ala-Ser-Chg-Gln-Ser-Leu-Dox Sodium salt (Compound 16) (SEQ.ID.NO.: 25)

The lyophalized Compound 15 free acid (2.0 g, 1.43 mmol), prepared as described in Example 5, was dissolved in 10 mL of water and a 0.100 N aqueous NaOH solution (14.3 mL, 1.43 mmol) was added over 10 min. with vigorous stirring. The pH of the solution at the end of the addition was 6.3. The water was removed by evaporation under a nitrogen stream to provide a microcrystalline solid.

Alternatively, addition of acetone to the aqueous solution of the sodium salt resulted in precipitation of the compound from solution. The salt was collected by filtration and dried under a nitrogen stream. The solid was recrystallized from 1:12 water acetone to provide a microcrytalline solid.

Step 19A: Alternative Preparation of [N-Glutaryl-(4-trans-L-Hyp)]-Ala-Ser-Chg-Gln-Ser-Leu-Dox Sodium salt (Compound 16) (SEQ.ID.NO.: 25)

The compound 4 piperidine salt (10.37 g, 71% by wt free acid), prepared as described in Example 5, was dissolved in acetone (50 mL) and sodium acetate buffer (pH 5.2 0.2 M, 50 mL), and then stirred at 21-22° C. for 1 h. Acetone was then added (150 mL) slowly over 45 mins. The solution was then seeded with Compound 5 (50 mg) and the batch aged for 1 h at 21-22° C. Acetone (100 mL) was then added slowly over 2h. The suspension was then cooled to 5° C. over 30 mins, and aged at 2-5° C. for 1 h. The product was isolated by filtration under an atmosphere of nitrogen, and the filter cake washed with 9:1 acetone/water (70 mL) followed by acetone (35 mL). The product was dried on the filter, under an atmosphere of nitrogen, overnight to give the sodium salt as a white crystalline solid.

Step 19B: Alternative Preparation of [N-Glutaryl-(4-trans-L-Hyp)]-Ala-Ser-Chg-Gln-Ser-Leu-Dox Sodium salt (Compound 16) (SEQ.ID.NO.: 25)

Compound 13 (0.91 g) was added to a 250 mL three necked flask, and was dissolved in dry DMF (15 mL). The solution was degassed twice and then cooled to 0° C. 1.91 mL of the 1.0 M piperidine in DMF was added over 60 minutes with a syringe pump. The solution was aged until disappearance of the Compound 13 was seen by HPLC (˜125 min).

250 μL glacial acetic acid (6.9 eq) was then added over 10 minutes in order to keep the temperature below 5° C. 740 μL of 2 M NaOAc (2.33 eq) was then added to the solution.

Acetone (132 mL) was added slowly, however after addition of the first 30 mL a precipitate was seen. After addition of 50 mL of acetone, the mixture was seeded with 20 mg of Compound 5. The solution was aged for 30 minutes, and then the remaining acetone was added over 60 minutes, while maintaining the temperature below 5° C. The solid was filtered through a 60 mL medium sintered glass funnel, and the solid was washed with 10 mL 9:1 acetone: water. It is allowed to dry with vacuum, with a nitrogen tent to provide Compound 16 as a solid.

### Example 13

### Preparation of (4-trans-L-Hyp)-Ala-Ser-Chg-Gln-Ser-Leu-Dox (SEQ.ID.NO.: 24)

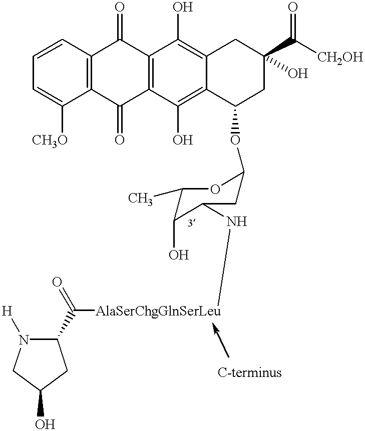

Step A: Fmoc-(4-trans-L-Hyp(Bzl))-Ala-Ser(Bzl)Chg-Gln-Ser(Bzl)Leu-PAM Resin

Starting with 0.5 mmol (0.67 g) Boc-Leu-PAM resin, the protected peptide was synthesized on a 430A ABI peptide synthesizer. The protocol used a 4 fold excess (2 mmol) of each of the following protected amino acids: Boc-Ser(Bzl), Boc-Gln, Boc-Chg, Boc-Ala, N-Boc-(4-trans-L-Hyp(Bzl)). Coupling was achieved using DCC and HOBT activation in methyl-2-pyrrolidinone. Fmoc-OSu (succinamidyl ester of Fmoc) was used for the introduction of the N-terminal protecting group. Removal of the Boc group was performed using 50% TFA in methylene chloride and the TFA salt neutralized with diisopropylethylamine. At the completion of the synthesis the peptide resin was dried to yield the title intermediate.

Step B: Fmoc-(4-trans-L-Hyp)-Ala-Ser-Chg-Gln-Ser-Leu-OH

The protected peptide resin from Step A, 1.1 g, was treated with HF (20 ml) for 1 hr at 0° C. in the presence of anisole (2 ml). After evaporation of the HF, the residue was washed with ether, filtered and extracted with H₂O (200 ml). The filtrate was lyophilyzed to yield the title intermediate.

Step C: Fmoc-(4-trans-L-Hyp)-Ala-Ser-Chg-Gln-Ser-Leu-Dox

The intermediate from Step B, 0.274 g, was dissolved in DMSO (10 ml) and diluted with DMF (10 ml). To the solution was added doxorubicin hydrochloride, 104 mg followed by 62 μL of diisopropylethylamine (DIEA). The stirred solution was cooled (0° C.) and 56 μL of diphenylphosphoryl azide added. After 30 minutes, an additional 56 μL of DPPA was added and the pH adjusted to ˜7.5 (pH paper) with DIEA. The pH of the cooled reaction (0° C.) was maintained at ˜7.5 with DIEA. After 4 hrs., the reaction (found to be complete by analytical HPLC, system A) was concentrated to an oil. HPLC conditions, system A.

Step D: (4-trans-L-Hyp)-Ala-Ser-Chg-Gln-Ser-Leu-Dox

The above product from Step C was dissolved in DMF (10 mL), cooled (0° C.) and 4 mL of piperidine added. The solution was concentrated to dryness and purified by preparative HPLC. (A=0.1% NH₄OAc—H₂O ; B=CH₃CN.) The crude product was dissolved in 100 mL of 90% A buffer, filtered and purified on a C-18 reverse phase HPLC radial compression column (Waters, Delta-Pak, 15μ, 100 é). A step gradient of 90% A to 65% A was used at a flow rate of 75 mL/min (uv=214 nm). Homogeneous product fractions (evaluated by HPLC, system A) were pooled and freeze-dried.

| | |
|---|---|
| Molecular Formula: | C₆₀H₈₃N₉O₂₂ |
| Molecular Weight: | 1281.56 |
| High Resolution ES Mass Spec: | 1282.59 (MH⁺) |
| HPLC: | System A |
| Column: | Vydac 15 cm #218TP5415, C18 |
| Eluant: | Gradient 95:5 (A:B) to 5:95 |
| | (A:B) over 45 min. A = 0.1% |
| | TFA/H₂O, B = 0.1% |
| | TFA/Acetonitrile |
| Flow: | 1.5 ml/min. |
| Wavelength: | 214 nm, 254 nm |
| Retention Time: | 17.6 min. |

Amino Acid Compositional Analysis¹:

| Theory | Found |
|---|---|
| Ala (1) | 1.00 |
| Ser (2) | 1.94 |
| Chg (1) | 0.94 |
| Gln² (1) | 1.05 (as Glu) |
| Hyp (1) | 0.96 |
| Leu (1) | 1.03 |
| Peptide Content: | 0.690 μmol/mg |

Note:
¹20 hr., 100° C., 6N HCl
²Gln converted to Glu

### Example 14

### des-Acetylvinblastine-4-O-(N-Acetyl-4-trans-L-Hyp-Ser-Ser-Chg-Gln-Ser-Ser-Pro) ester (SEQ.ID.NO.: 36)

Step A: Preparation of 4-des-Acetylvinblastine

A sample of 2.40 g (2.63 mmol) of vinblastine sulfate (Sigma V-1377) was dissolved under N₂ in 135 mL of absolute methanol and treated with 45 mL of anhydrous hydrazine, and the solution was stirred at 20-25° C. for 18 hr. The reaction was evaporated to a thick paste, which was partitioned between 300 mL of CH₂Cl₂ and 150 mL of saturated NaHCO₃. The aqueous layer was washed with 2 100-ml portions of CH₂Cl₂, and each of the 3 CH₂Cl₂ layers in turn was washed with 100 mL each of H₂O (2×) and saturated NaCl (1×). The combined organic layers were dried over anhydrous Na₂SO₄, and the solvent was removed at reduced pressure to yield the title compound as an off-white crystalline solid. This material was stored at −20° C. until use.

Step B: Preparation of 4-des-Acetylvinblastine 4-O-(Prolyl) ester

A sample of 804 mg (1.047 mmol) of 4-des-acetylvinblastine, dissolved in 3 mL of CH₂Cl₂ and 18 mL of anhydrous pyridine under nitrogen, was treated with 1.39 g of Fmoc-proline acid chloride (Fmoc-Pro-Cl, Advanced Chemtech), and the mixture was stirred for 20 hr at 25° C. When analysis by HPLC revealed the presence of unreacted starting des-acetylvinblastine, another 0.50 g of Fmoc-Pro-Cl was added, with stirring another 20 hr to complete the reaction. Water (ca. 3 ml) was added to react with the excess acid chloride, and the solution was then evaporated to dryness and partitioned between 300 mL of EtOAc and 150 mL of saturated NaHCO₃, followed by washing twice with saturated NaCl. After drying (Na₂SO₄), the solvent was removed under reduced pressure to give an orange-brown residue, to which was added 30 mL of DMF and 14 mL of piperidine, and after 5 min the solution was evaporated under reduced pressure to give a orange-yellow semi-solid residue. After drying in vacuo for about 1 hr, approx. 200 mL of H₂O and 100 mL of ether was added to this material, followed by glacial HOAc dropwise with shaking and sonication until complete dissolution had occurred and the aqueous layer had attained a stable pH of 4.5-5.0 (moistened pH range 4-6 paper). The aqueous layer was then washed with 1 100-ml portion of ether, and each ether layer was washed in turn with 50 mL of H₂O. The combined aqueous layers were subjected to preparative HPLC in 2 portions on a Waters C4 Delta-Pak column 15 μM 300A (A=0.1% TFA/H₂O; B=0.1% TFA/CH₃CN), gradient elution 95→70% A/70 min. Pooled fractions yielded, upon concentration and lyophilization, the title compound.

Step C: N-Acetyl-4-trans-L-Hyp-Ser-Ser-Chg-Gln-Ser-Ser-WANG Resin (SEQ.ID.NO.: 50)

Starting with 0.5 mmole (0.61 g) of Fmoc-Ser(t-Bu)-WANG resin loaded at 0.82 mmol/g, the protected peptide was synthesized on a ABI model 430A peptide synthesizer adapted for Fmoc/t-butyl-based synthesis. The protocol used a 2-fold excess (1.0 mmol) of each of the following protected amino acids: Fmoc-Ser(t-Bu)-OH, Fmoc-Gln-OH, Fmoc-Chg-OH, Fmoc-4-trans-L-Hyp-OH; and acetic acid (double coupling). During each coupling cycle Fmoc protection was removed using 20% piperidine in N-methyl-2-pyrrolidinone (NMP), followed by washing with NMP. Coupling was achieved using DCC and HOBt activation in NMP. At the completion of the synthesis, the peptide resin was dried to yield the title compound.

Step D: N-Acetyl-4-trans-L-Hyp-Ser-Ser-Chg-Gln-Ser-Ser-OH(SEQ.ID.NO.: 50)

One 0.5-mmol run of the above peptide-resin was suspended in 25 mL of TFA, followed by addition of 0.625 mL each of H₂O and triisopropylsilane, then stirring at 25° for 2.0 hr. The cleavage mixture was filtered, the solids were washed with TFA, the solvents were removed from the filtrate under reduced pressure, and the residue was triturated with ether to give a pale yellow solid, which was isolated by filtration and drying in vacuo to afford the title compound.

HPLC conditions, system A:

Column . . . Vydac 15 cm #218TP5415, C18

Eluant . . . Gradient (95% A →50% A) over 45 min. A=0.1% TFA/H₂O, B=0.1% TFA/acetonitrile

Flow . . . 1.5 ml/min.

High Resolution ES/FT-MS: 789.3

Step E: des-Acetylvinblastine-4-O-(N-Acetyl-4-trans-L-Hyp-Ser-Ser-Chg-Gln-Ser-Ser-Pro) ester

Samples of 522 mg (0.66 mmol) of the peptide prepared as described in step D and 555 mg (ca. 0.6 mmol) of 4-des-Acetylvinblastine 4-O-(Prolyl) ester from Step B, prepared as above, were dissolved in 17 mL of DMF under N₂. Then 163 mg (1.13 mmol) of 1-hydroxy-7-azabenzotriazole (HOAt) was added, and the pH was adjusted to 6.5-7 (moistened 5-10 range pH paper) with 2,4,6-collidine, followed by cooling to 0° C. and addition of 155 mg (0.81 mmol) of 1-(3-dimethylaminopropyl)-3-ethylcarbodiimide hydrochloride (EDC). Stirring was continued at 0-5° C. until completion of the coupling as monitored by analytical HPLC (A=0.1% TFA/H₂O; B=0.1% TFA/CH₃CN), maintaining the pH at 6.5-7 by periodic addition of 2,4,6-collidine. After 12 hr the reaction was worked up by addition of ˜4 mL of H₂O and, after stirring 1 hr, concentrated to a small volume in vacuo and dissolution in ca. 150 mL of 5% HOAc and preparative HPLC in two portions on a Waters C₁₈ Delta-Pak column 15 μM 300A (A=0.1% TFA/H2O; B=0.1% TFA/CH₃CN, gradient elution 95→65% A/70 min). Homogeneous fractions containing the later-eluting product (evaluated by HPLC, system A, 95→65% A/30 min) from both runs were pooled and concentrated to a volume of ˜50 mL and passed through approx. 40 mL of AG4X4 ion exchange resin (acetate cycle), followed by freeze-drying to give the title compound as a lyophilized powder.

High Resolution ES/FT-MS: 1637.0

### EXAMPLE 15

### des-Acetylvinblastine-4-O-(N-Acetyl-4-trans-L-Hyp-Ser-Ser-Chg-Gln-Ser-Ser-Pro) ester acetate

A sample of 4.50 g (3.7 mmol) of 4-O-(prolyl) des-acetylvinblastine TFA salt, prepared as described in Example 14, Step B, was dissolved in 300 mL of DMF under N₂, and the solution was cooled to 0° C. Then 1.72 g (10.5 mmol) of 3,4-dihydro-3-hydroxy-4-oxo-1,2,3-benzotriazine (ODHBT) was added, and the pH was adjusted to 7.0 (moistened 5-10 range pH paper) with N-methylmorpholine (NMM), followed by the addition of 4.95 g (5.23 mmol) of the N-acetyl-heptapeptide of Example 28, Step D, portionwise allowing complete dissolution between each addition. The pH was again adjusted to 7.0 with NMM, and 1.88 g (9.8 mmol) of 1-(3-dimethylaminopropyl)-3-ethylcarbodiimide hydrochloride (EDC) was added, followed by stirring of the solution at 0-5° C. until completion of the coupling as monitored by analytical HPLC (system A), maintaining the pH at ca. 7 by periodic addition of NMM. The analysis showed the major component at 26.3 min retention time preceded by a minor component (ca. 10%) at 26.1 min, identified as the D Ser isomer of the title compound. After 20 hr the reaction was worked up by addition of 30 mL of H₂O and, after stirring 1 hr, concentrated to a small volume in vacuo and dissolution in ca. 500 mL of 20% HOAc. and preparative HPLC in 12 portions on a Waters C₁₈ Delta-Pak column 15mM 300A (A=0.1% TFA/H₂O; B=0.1% TFA/CH₃CN), gradient elution 85→65% A/90 min) at a flow rate of 80 ml/min.

Homogeneous fractions (evaluated by HPLC, system C) representing approx. one-fourth of the total run were pooled and concentrated to a volume of ˜150 mL and passed through approx. 200 mL of Bio-Rad AG4X4 ion exchange resin (acetate cycle), followed by freeze-drying of the eluant gave the acetate salt of the title compound as a lyophilized powder: retention time (system A) 26.7 min, 98.9% pure; high resolution ES/FT-MS m/e 1636.82; amino acid compositional analysis 20 hr, 100° C., 6N HCl (theory/found), Ser4/3.91 (corrected), Glu 1/0.92 (Gln converted to Glu), Chg 1/1.11, Hyp 1/1.07, Pro 1/0.99, peptide content 0.516 mmol/mg.

Further combination of homogeneous fractions and purification from side fractions, processing as above through approx. 500 mL of ion exchange resin, afforded an additional amounts of the title compound.

HPLC conditions, system A:

Column . . . Vydac 15 cm #218TP5415, C18

Flow . . . 1.5 ml/min.

Eluant . . . Gradient (95% A→50% A) over 45 min.

A=0.1% TFA/H₂O, B=0.1% TFA/acetonitrile

Wavelength . . . 214 nm, 280 nm

HPLC conditions, system C:

Column . . . Vydac 15 cm #218TP5415, C18

Flow . . . 1.5 ml/min.

Eluant . . . Gradient (85% A→65% A) over 30 min.

A=0.1% TFA/H₂O, B=0.1% TFA/acetonitrile

Wavelenth . . . 214 nm, 280 nm

### Example 16

### Preparation of 4-des-Acetylvinblastine-23-(4′-aminomethylbicyclo-[2.2.2]octane) methylamide (BDAM-(dAc)vinblastine)

Step A Preparation of 4-des-Acetylvinblastine-23-hydrazide

A sample of 3.99 g (4.38 mmol) of vinblastine sulfate (Sigma V-1377) was dissolved in 30.4 mL of 1:1 (v/v) absolute ethanol/anhydrous hydrazine, under N₂, and the solution was heated in an oil bath at 60-65° C. for 23 hr. Upon cooling, the solution was evaporated to a thick paste, which was partitioned between 300 ml of CH₂Cl₂ and 150 mL of saturated NaHCO₃. The aqueous layer was washed with 2 100-ml portions of CH₂Cl₂, and each of the 3 CH₂Cl₂ layers in turn was washed with 100 mL each of H₂O (2×) and saturated NaCl (1×). The combined organic layers were dried over anhydrous Na₂SO₄, and the solvent was removed in vacuo to yield, after drying 20 hr in vacuo, the title compound as a white crystalline solid. This material was dissolved in 82 mL of dry, degassed DMF for storage at ˜20° C. until use (conc. 36 mg/ml).

Step B Boc-4-aminomethylbicyclo-[2.2.2]octane carboxylic acid

A sample of 8.79 g (40.0 mmol) of 4-carboxybicyclo-[2.2.2]octanemethylamine hydrochloride salt suspended in 100 mL each of THF and H₂O was treated with 20.0 mL (14.6 g=3.3 equiv.) of TEA, followed by 11.8 g (47.9 mmol) of BOC-ON reagent. All went into solution, and after stirring 24 hr the solution was concentrated in vacuo to a volume of about 50 mL and partitioned between 100 mL of ether and 300 mL of H₂O. After addition of about 2 mL of TEA the aqueous layer was washed with ether (3×), each ether in turn washed with H₂O, and the combined aqueous layer was acidified with 5% KHSO₄ to give the title compound as a white solid, isolated by filtration and drying in vacuo.

Step C Boc-4-aminomethylbicyclo-[2.2.2]octane carboxamide

A stirred solution under N₂ of 12.0 g (42.5 mmol) of the product from step B in 100 mL of DMF was treated with 8.0 g (49.3 mmol) of carbonyldiimidazole. After 30 min the DMF was evaporated in vacuo to afford 50-60 mL of a light brown paste, which was stirred and treated with 70 mL of conc. NH₄OH rapidly added. The initial solution turned to a white paste within 30 min, after which H₂O was added up to a total volume of 400 mL to complete precipitation of product, which was triturated and isolated by filtration and washing with H₂O, and dried in vacuo to yield the title compound as a white solid.

Step D Boc-4-aminomethylbicyclo-[2.2.2]octane nitrile

A solution of 7.52 g (26.6 mmol) of the product from step C in 50 mL of CH₂Cl₂ and 80 mL of anhydrous pyridine was treated with 11.12 g of (methoxycarbonylsulfamoyl)-triethyl-ammonium hydroxide inner salt (Burgess reagent) in 1-g portions over 5 min. After stirring for 1.5 hr, TLC (90-10-1, CHCl₃—CH₃OH—H₂O) showed complete conversion to product, and the solution was evaporated to give a paste, to which H₂O was added, up to 400 ml, with trituration and stirring to afford, after standing 20 hr at 0° C., filtration and drying in vacuo, the title compound as a white solid.

Step E Boc-4-aminomethylbicyclo-[2.2.2]octane methylamine

A solution of 6.75 g (25.5 mmol) of the product from step D in 200 mL of CH₃OH plus 4 mL of HOAc and 2 mL of H₂O was hydrogenated over 1.63 g of PtO₂ in a Parr shaker at 55 psi for 22 hr. The catalyst was removed by filtration through Celite, and the filtrate was concentrated in vacuo to an oily residue, which was flushed/evaporated with CH₃OH (1×) and CH₂Cl₂ (2×). Product began to crystallize toward the end of the evaporation, and ether (up to 300 ml) was added to complete the precipitation. The white solid was triturated and isolated by filtration and washing with ether to give, after drying in vacuo, the title compound as the acetate salt.

400 Mhz ¹H-NMR (CDCl₃): δ(ppm, TMS) 4.5 (1s, Boc-NH); 2.9 (2br d, —CH₂—NH-Boc); 2.45 (2br s, —CH₂—NH₂); 2.03 (3s, CH₃COOH);1.45 (9s, Boc); 1.40 (12s, ring CH₂).

Step F Preparation of 4-des-Acetylvinblastine-23-(4′-aminomethylbicyclo-[2.2.2]octane) methylamide (BDAM-(dAc)vinblastine)

A 30-ml aliquot of the above DMF solution of 4-des-acetylvinblastine-23-hydrazide (1.41 mmol), cooled to −15° C. under Argon, was converted to the azide in situ by acidification with 4M HCl in dioxane to pH<1.5 (moistened 0-2.5 range paper), followed by addition of 0.27 mL (1.3 equiv) of isoamyl nitrite and stirring for 1 hr at 10-15° C. The pH was brought to 7 by the addition of DIEA, and a slurry of 1.27 g (3.8 mmol) of the Boc diamine product from step E above in 20 mL of DMF was then added, and the reaction was allowed to warm slowly to 15-20° C. over 2 hr, at which point coupling was complete, as monitored by analytical HPLC (A=0.1% TFA/H₂O; B=0.1% TFA/CH₃CN). The solvent was removed in vacuo and the residue partitioned between EtOAc and 5% NaHCO₃, the organic layer washed with 5% NaCl, and the aqueous layers back-extracted with CH₂Cl₂ to assure removal of the intermediary Boc-BDAM-(dAc)vinblastine. The combined organic layers were dried over Na₂SO₄, the solvent was removed under reduced pressure, and the residue, after flush/evaporation twice from CH₂Cl₂, was dissolved in 30 mL of CH₂Cl₂ and treated with 30 mL of TFA for 30 min. The solvents were rapidly removed in vacuo, and the residue was dissolved in 300 mL of 10% HOAc for purification by preparative HPLC in 5 portions on a Waters C4 Delta-Pak column 15 μM 300A (A=0.1% TFA/H₂O; B=0.1% TFA/CH₃CN), gradient elution 95→70% A/60 min, isocratic 70%/20 min. Homogeneous fractions (evaluated by HPLC, system A, 95→50% A) from the five runs were pooled and concentrated in vacuo, followed by freeze-drying to give of the title compound as the lyophilized TFA salt.

HPLC conditions, system A:

Column . . . Vydac 15 cm #218TP5415, C18

Eluant . . . Gradient (A→B) over 45 min.

A=0.1% TFA/H₂O, B=0.1% TFA/acetonitrile

Flow . . . 1.5 ml/min.

Retention time: BDAM (dAc) vinblastine 23.5 min. (95%→50% A) 97% purity

High Resolution ES/FT-MS: 905.63

Compound content by elemental analysis=0.714 μmol/mg:

N (calc)=9.28 N (found)=6.00

### Example 17

### Preparation of 4-des-Acetylvinblastine-23-(N-Acetyl-Ser-Ser-Ser-Chg-Gln-Ser-Val-BDAM) amide acetate salt (SEQ.ID.NO.: 32)

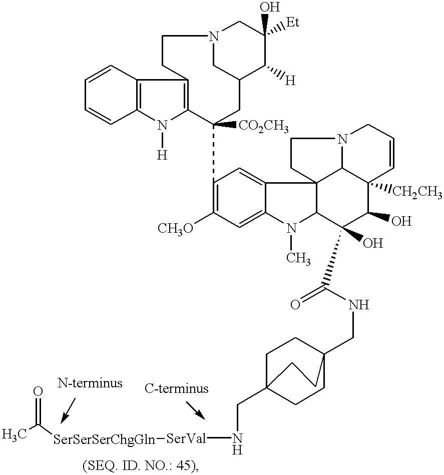

Step A: N-Acetyl-Ser-Ser-Ser-Chg-Gln-Ser-Val-PAM Resin (SEQ.ID.NO.:32)

Starting with 0.5 mmole (0.68 g) of Boc-Val-PAM resin, the protected peptide was synthesized on a ABI model 430A peptide synthesizer. The protocol used a 4-fold excess (2.0 mmol) of each of the following protected amino acids: Boc-Ser(Bzl)-OH, Boc-Gln-OH, Boc-Chg-OH; and acetic acid (2 couplings). During each coupling cycle Boc protection was removed using TFA, followed by neutralization with DIEA. Coupling was achieved using DCC and HOBt activation in N-methyl-2-pyrrolidinone. At the completion of the synthesis, the peptide resin was dried to yield the title compound.

Step B: N-Acetyl-Ser-Ser-Ser-Chg-Gln-Ser-Val-OH (SEQ.ID.NO.: 32)

Three 0.5-mmol runs of the above peptide-resin (3.5 g) were combined and treated with liquid HF (65 ml) for 1.5 hr at 0° C. in the presence of anisole (6 ml). After evaporation of the HF, the residue was washed with ether, filtered and leached with 150 mL of DMF in several portions, adding DIEA to pH ˜8, followed by removal of the DMF in vacuo to a volume of 100 ml. The concentration was determined as ca. 11.7 mg/ml (by weighing the dried resin before and after leaching. The sample purity was determined as 96% by HPLC. The solution was used directly for conjugation with BDAM-(dAc)vinblastine.

Step C: 4-Des-acetylvinblastine-23-(N-Acetyl-Ser-Ser-Ser-Chg-Gln-Ser-Val-BDAM) amide acetate salt

To 58 mL (equivalent to 0.875 mmol of peptide) of the solution from step B was added 530 mg (0.520 mmol) of BDAM-(dAc)vinblastine, prepared as described in Example 30, Step F, under N₂, cooling to 0° C., and the pH was adjusted to ˜8 (moistened 5-10 range pH paper) with DIEA. Then 0.134 mL (0.62 mmol) of DPPA was added, followed by stirring at 0-5° C. until completion of the coupling as monitored by analytical HPLC (A=0.1% TFA/H₂O; B=0.1% TFA/CH₃CN), maintaining the pH at ≧7 by periodic addition of DIEA. After 24 hr, the reaction was worked up by addition of 10 mL of H₂O, stirring 1 hr and concentration to small volume in vacuo, then dissolution in ca. 100 mL of 10% HOAc/5% CH₃CN, adjustment of the pH to 5 with NH₄HCO₃, filtration to remove insolubles, and preparative HPLC in 3 portions on a Waters C4 Delta-Pak column 15 μM 300A (A=0.1% NH₄HCO₃/H₂O; B=CH₃CN), gradient elution 95→40% A/70 min. Fractions from each run containing product were pooled, acidified to pH 3 with glacial HOAc, concentrated in vacuo to a volume of ˜50 ml, and purified by preparative HPLC on a Waters C18 Delta-Pak column 15 μM 300A (A=0.1% TFA/H₂O; B=0.1% TFA/CH₃CN), gradient elution 95→70% A/60 min, isocratic 70%/20 min. Homogeneous fractions (evaluated by HPLC, system A, 95→50% A) from all three runs were pooled and concentrated to a volume of ˜100 ml., diluted with 5% CH₃CN, and passed through AG4X4 ion exchange resin (acetate cycle), followed by freeze-drying to give the title compound as a lyophilized powder.

| | |
|---|---|
| HPLC conditions, system A: | |
| Column... | Vydac 15 cm #218TP5415, |
| | C18 |
| Eluant... | Gradient (A --> B) over |
| | 45 min. A = 0.1% TFA/ |
| | H₂O, B = 0.1% |
| | TFA/acetonitrile |
| Flow... | 1.5 ml/min. |
| Retention times: | BDAM (dAc) vinbiastine |
| | 23.5 min. |
| N-Acetyl-Ser-Ser-Ser-Chg-Gln-Ser-Val-OH | 14.5 min. |
| 4-Des-acetylvinblastine-23- | 29.5 min. |
| (N-Acetyl-Ser-Ser-Ser-Chg- | |
| Gln-Ser-Val-BDAM) amide | |
| High Resolution ES/FT-MS: | 1662.03 |
| Amino Acid Compositional | |

Analysis¹ (theory/found):

| | | | |
|---|---|---|---|
| ²Ser4/3.6 | ³Glu 1/2.10 | ⁴Val 1/0.7 | Chg 1/0.95 |

| | |
|---|---|
| Peptide content | 0.504 μmol/mg |

Note:
¹20 hr, 100° C., 6N HCl
²Uncorrected
³Gln converted to Glu
⁴Incomplete hydrolysis

### Example 18

### Preparation of 4-des-Acetylvinblastine-23-(N-methoxy-diethylene-oxyacetyl-4-trans-L-Hyp-Ser-Ser-Chg-Gln-Ser-Val-BDAM) amide acetate salt (SEQ.ID.NO.: 33)

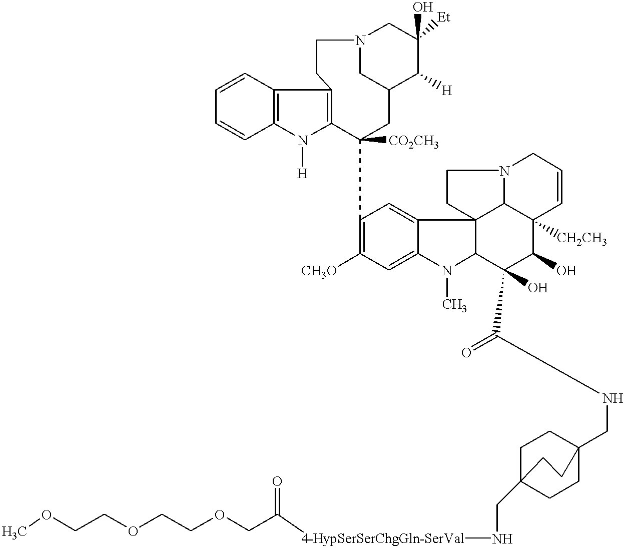

### (SEQ.ID.NO.: 33)

Step A: N-methoxydiethyleneoxyacetyl-4-trans-L-Hyp-Ser-Ser-Chg-Gln-Ser-Val-PAM Resin (SEQ.ID.NO.: 33)

Starting with 0.5 mmole (0.68 g) of Boc-Val-PAM resin, the protected peptide was synthesized on a ABI model 430A peptide synthesizer. The protocol used a 4-fold excess (2.0 mmol) of each of the following protected amino acids: Boc-Ser(Bzl)-OH, Boc-Gln-OH, Boc-Chg-OH, Boc-4-trans-Hyp(Bzl)-OH; and 2-[2-(2-methoxyethoxy)-ethoxy]acetic acid (2 couplings). During each coupling cycle Boc protection was removed using TFA, followed by neutralization with DIEA. Coupling was achieved using DCC and HOBt activation in N-methyl-2-pyrrolidinone. At the completion of the synthesis, the peptide resin was dried to yield the title compound.

Step B: N-methoxydiethyleneoxyacetyl-4-trans-L-Hyp-Ser-Ser-Chg-Gln-Ser-Val-OH (SEQ.ID.NO.: 33)

Two 0.5-mmol runs of the above peptide-resin (2.4 g) were combined and treated with liquid HF (40 ml) for 1.5 hr at 0° C. in the presence of anisole (4 ml). After evaporation of the HF, the residue was washed with ether, filtered and leached with 150 mL of H₂O in several portions, followed by preparative HPLC on a Waters C18 Delta-Pak column 15 μM 100A (A=0.1% TFA/H₂O; B=0.1% TFA/CH₃CN), gradient elution 95→70% A/70 min, and pooling of homogeneous fractions and freeze drying to give the title compound as lyophilized powder. The sample purity was determined as 99% by HPLC.

Step C: 4-des-Acetylvinblastine-23-(N-methoxydiethylene-oxyacetyl-4-trans-L-Hyp-Ser-Ser-Chg-Gln-Ser-Val-BDAM) amide acetate salt

Samples of 440 mg (0.47 mmol) of the peptide from step B and 340 mg (0.33 mmol) of BDAM-(dAc)vinblastine, prepared as described in Example 30, Step F, were dissolved in 25 mL of DMF under N₂, cooling to 0° C. Then 85 mg (0.63 mmol) of 1-hydroxy-7-azabenzotriazole (HOAt) was added, and the pH was adjusted to 6.5-7 (moistened 5-10 range pH paper) with 2,4,6-collidine, followed by addition of 117 mg (0.61 mmol) of 1-(3-dimethylaminopropyl)-3-ethylcarbodiimide hydrochloride (EDC). Stirring was continued at 0-5° C. until completion of the coupling as monitored by analytical HPLC (A=0.1% TFA/H₂O; B=0.1% TFA/CH₃CN), maintaining the pH at 6.5-7 by periodic addition of 2,4,6-collidine. After 3 hr the reaction was worked up by addition of ˜10 mL of H₂O, stirring 1 hr and concentration to small volume in vacuo, then dissolution in ca. 70 mL of 5% HOAc and preparative HPLC on a Waters C18 Delta-Pak column 15 μM 300A (A=0.1% TFA/H₂O; B=0.1% TFA/CH₃CN), gradient elution 95→40% A/70 min). Homogeneous fractions (evaluated by HPLC, system A, 95→50% A) from all three runs were pooled and concentrated to a volume of ˜50 mL and passed through AG4X4 ion exchange resin (acetate cycle), followed by freeze-drying to give the title compound as a lyophilized powder.

| HPLC conditions, system A: | |
|---|---|
| Column... | Vydac 15 cm #218TP5415, C18 |
| Eluant... | Gradient (A --> B) over 45 min. |
| | A = 0.1% TFA/H₂O, B = 0.1% |
| | TFA/acetonitrile |
| Flow... | 1.5 ml/min. |
| Retention times: | BDAM (dAc) vinblastine |
| | 23.5 min. |
| N-methoxydiethyleneoxyacetyl- | 16.2 min. |
| 4-trans-L-Hyp-Ser-Ser-Chg- | |
| Gln-Ser-Val-OH | |
| 4-des-Acetylvinblastine-23- | 29.6 min. |
| (N-methoxydiethyleneoxyacetyl- | |
| 4-trans-L-Hyp-Ser-Ser-Chg-Gln- | |
| Ser-Val-BDAM) amide | |
| High Resolution ES/FT-MS: | 1805.95 |

Amino Acid Compositional Analysis¹ (theory/found):

| | | | |
|---|---|---|---|
| ²Ser3/1.7 | ³Glu 1/1.01 | ⁴Val 1/0.93 | Chg 1/0.98 |
| Hyp 1/1.01 | | | |

| | |
|---|---|
| Peptide content = | 0.497 μmol/mg |

Note:
¹20 hr, 100° C., 6N HCl
²Uncorrected
³Gln converted to Glu
⁴Incomplete hydrolysis

### Example 18

### Preparation of 4-des-Acetylvinblastine-23-(N-Acetyl-4-trans-L-Hyp-Ser-Ser-Chg-Gln-Ser-HCAP) amide acetate salt (18-7)

Step A: N-Acetyl-4-trans-L-Hyp-Ser-Ser-Chg-Gln-OH (18-1) (SEQ.ID.NO. 50)

Starting with 0.5 mmole (0.80 g) of Fmoc-Gln(Trt)-Wang resin, the protected peptide was synthesized on a ABI model 430A peptide synthesizer. The protocol used a 4-fold excess (2.0 mmol) of each of the following protected amino acids: Fmoc-Ser(tBu)-OH, Fmoc-Chg-OH, Fmoc-4-trans-Hyp(tBu)-OH and acetic acid (2 couplings). During each coupling cycle Fmoc protection was removed using 20% piperidine in DMF. Coupling was achieved using DCC and HOBt activation in N-methyl-2-pyrrolidinone. At the completion of the synthesis, the peptide resin was dried. 1.3 g peptide-resin was treated with 95% TFA: 2.5% H2O: 2.5% Triisopropylsilane (20 ml) for 2 hr at r.t. under argon. After evaporation of the TFA, the residue was washed with ether, filtered and dried to give crude peptide which was purified by preparatory HPLC on a Delta-Pak C18 column with 0.1% trifluoroacetic acid aqueous acetonitrile solvent systems using 100 70% A, 60 min linear gradient. Fractions containing product of at least 99% (HPLC) purity were combined to give the title compound.

FABMS: 615.3

Peptide Content: 1.03 nmole/mg.

HPLC: 99% pure @214 nm, retention time=10.16 min, (Vydac C₁₈, gradient of 95% A/B to 50% A/B over 30 min, A=0.1% TFA-H₂O, B=0.1% TFA-CH₃CN)

Step B: N-Boc-(1S,2R)-(+)-Norephedrine (18-2)

A solution of 1.51 g (10 mmol) of (1S,2R)-(+)-Norephedrine in a mixture of 1,4-dioxane (20 ml), water (10 ml) and 1N NaOH (10 ml) was stirred and cooled in an ice-water bath. Di-(t-butyl) dicarbonate (2.4 g, 11 mmol) was added in portions over approx. 20 min. The reaction was stirred in the cold for 2 hrs., then at room temp. for an additional 1 h. The solution was concentrated to remove most of the dioxane, cooled in an ice bath and covered with a layer of ethyl acetate (30 ml) and acidified to pH 2 with 1N KHSO₄. The aqueous phase was extracted 2× with EtOAc. The combined extracts were washed with water, brine and were concentrated and dried to provide the desired product as a white crystalline solid (18-2). FABMS: 252

Step C: N-Boc-HCAP (18-3)

A solution of 2.38 g of N-Boc-(1S,2R)-(+)-Norephedrine (18-2) in 50 mL acetic acid/10 mL H₂O was hydrogenated at 60 psi on a Parr apparatus over 500 mg of Ir black catalyst for 24 hrs. The reaction was filtered through a Celite pad, and the filtrate concentrated in vacuo to give a tan foam (18-3). FABMS: 258.2

Step D: N-Benzyloxycarbonyl-Ser-N-t-Boc-HCAP ester (2-4)

A solution of 1.95 g (6.6 mmol) of N-Z-Ser(tBu)-OH, 1.54 g (6.0 mmol) of N-Boc-HCAP (18-3), 1.26 g (6.6 mmol) of EDC, and 146 mg (1.2 mmol) of DMAP in 30 mL of anh. CH2C12 was treated and the resulting solution stirred at room temp. in an N₂ atmosphere for 12h. The solvent was removed in vacuo, the residue dissolved in ethyl acetate (150 ml) and the solution extracted with 0.5 N NaHCO₃ (50 ml), water (50 ml) and brine, then dried and concentrated to provide the crude coupling product (18-4).

Step E: H-Ser(tBu)-N-t-Boc-HCAP ester (18-5)

A 2.0 g of (18-4) in a solution of 90 mL EtOH, 20 ml water, and 10 mL acetic acid was hydrogenated on a Parr apparatus at 50 psi over 200 mg of Pd(OH)₂ catalyst for 3h. The reaction was filtered through a Celite pad, and the filtrate was concentrated to small volume in vacuo, then purified by preparatory HPLC on a Delta-Pak C18 column with 0.1% trifluoroacetic acid-aqueous acetonitrile solvent systems using 95-50% A, 60 min linear gradient. Fractions containing product of at least 99% (HPLC) purity were combined to give the intermediate (18-5). FABMS: 401.3

Step F: N-Acetyl-4-trans-L-Hyp-Ser-Ser-Chg-Gln-Ser-HCAP amine (18-6) (SEQ.ID.NO. 50)

A solution of 614 mg (1.0 mmol) of N-Acetyl-4-trans-L Hyp-Ser-Ser-Chg-Gln-OH (18-1), 400 mg (1.0 mmol) of H-Ser(tBu)-N-t-Boc-HCAP ester (18-5), 229 mg (1.2 mmol) of EDC, and 81 mg (0.5 mmol) of ODBHT (3,4-dihydro-3-hydroxy-4-oxo-1,2,3-benzotriazine), in 7 mL of DMF was stirred at 0° C. in an N₂ atmosphere for 10 h. The solvent was removed in vacuo, the residue was washed with ether and dried. The crude product was treated with 95% TFA: 5% H₂O (20 ml) for 2 hr at r.t. under argon. After evaporation of the TFA, the residue was purified by preparatory HPLC on a Delta-Pak C18 column with 0.1% trifluoroacetic acid -aqueous acetonitrile solvent systems using 95-50% A, 60min linear gradient. Fractions containing product of at least 99% (HPLC) purity were combined to give the intermediate compound (18-6).

FABMS: 841.8

Peptide Content: 863.39 NMole/mg.

HPLC: 99% pure @214 nm, retention time=13.7 min, (Vydac C₁₈, gradient of 95% A/B to 5% A/B over 30 min, A=0.1% TFA-H₂O, B=0.1% TFA-CH₃CN)

Step G: 4-des-Acetylvinblastine-23-(N-Ac-4-trans-L-Hyp-Ser-Ser-Chg-Gln-Ser-HCAP) amide acetate salt (18-7)

A solution of 0.461 of 4-des-acetylvinblastine-23-hydrazide (0.6 mmol) in 10 mL DMF cooled to −15° C. under Argon, was converted to the azide in situ by acidification with 4M HCl in dioxane to pH<1.5 (moistened 0-2.5 range paper), followed by addition of 0.105 mL (1.3 equiv) of isoamyl nitrite and stirring for 1 hr at 10-15° C. The pH was brought to 7 by the addition of DIEA, and 555 mg (0.66 mmol) of amine derivative (18-6) from step F was then added, and the reaction was stirred at 0° C. for 24 hrs, and purified by preparatory HPLC on a 15 μM,100A, Delta-Pak C18 column with 0.1% trifluoroacetic acid-aqueous acetonitrile solvent systems using 95-50% A, 60 min linear gradient. Homogeneous fractions were pooled and concentrated in vacuo, followed by freeze-drying to give the title compound as the TFA salt which was converted to the corresponding HOAc salt by AG 4×4 resin (100-200 mesh, free base form, BIO-RAD) (18-7).

ES⁺:1576.7

Peptide Content: 461.81 NMole/mg.

Ser 3.04; Hyp 1.07; Chg 1.02; Glu 1.00

HPLC: 99% pure @214 nm, retention time=18.31 min, (Vydac C₁₈, gradient of 95% A/B to 5% A/B over 30 min, A=0.1% TFA-H₂O, B=0.1% TFA-CH₃CN)

### Example 19

### Preparation of 4-des-Acetylvinblastine-23-(N-Acetyl-Ser-Chg-Gln-Ser-Ser-Pro-HCAP) amide acetate salt (19-7) (SEQ.ID.NO. 51)

Step A: N-Acetyl-Ser-Chg-Gln-Ser-Ser-OH (19-1)

Starting with 0.5 mmole (0.80 g) of Fmoc-Ser(tBu)-Wang resin, the protected peptide was synthesized on a ABI model 430A peptide synthesizer. The protocol used a 4-fold excess (2.0 mmol) of each of the following protected amino acids: Fmoc-Ser(tBu)-OH, Fmoc-Gln-OH, Fmoc-Chg-OH, Fmoc-Ser(tBu)-OH and acetic acid (2 couplings). During each coupling cycle Fmoc protection was removed using 20% piperidine in DMF. Coupling was achieved using DCC and HOBt activation in N-methyl-2-pyrrolidinone. At the completion of the synthesis, the peptide resin was dried. 1.3 g peptide-resin was treated with 95% TFA :2.5% H2O: 2.5% Triisopropylsilane (20 ml) for 2 hr at r.t. under argon. After evaporation of the TFA, the residue was washed with ether, filtered and dried to give crude peptide which was purified by preparatory HPLC on a Delta-Pak C18 column with 0.1% trifluoroacetic acid-aqueous acetonitrile solvent systems using 100-70% A, 60min linear gradient. Fractions containing product of at least 99% (HPLC) purity were combined to give the title compound.

FABMS: 589.5

Peptide Content: 1.01 NMole/mg.

HPLC: 99% pure @214 nm, retention time=10.7 min, (Vydac C₁₈, gradient of 95% A/B to 50% A/B over 30 min, A=0.1% TFA-H₂O, B=0.1% TFA-CH₃CN)

Step B: N-Boc-(1S,2R)-(+)-Norephedrine (19-2)

A solution of 1.51 g (10 mmol) of (1S,2R)-(+)-Norephedrine in a mixture of 1,4-dioxane (20 ml), water (10 ml) and 1N NaOH (10 ml) is stirred and cooled in an ice-water bath. Di-(t-butyl) dicarbonate (2.4 g, 11 mmol) was added in portions over approx. 20 min. The reaction was stirred in the cold for 2 hrs., then at room temp. for an additional 1 h. The solution was concentrated to remove most of the dioxane, cooled in an ice bath and covered with a layer of ethyl acetate (30 ml) and acidified to pH 2 with IN KHSO₄. The aqueous phase was extracted 2× with EtOAc. The combined extracts were washed with water, brine and were concentrated and dried to provide the desired product as a white crystalline solid. FABMS: 252

Step C: N-Boc-HCAP (19-3)

A solution of 2.38 g of N-Boc-(1S,2R)-(+)-Norephedrine (19-2) in 50 mL acetic acid/10 mL H₂O was hydrogenated at 60 psi on a Parr apparatus over 500 mg of Ir black catalyst for 24 hrs. The reaction was filtered through a Celite pad, and the filtrate concentrated in vacuo to give a tan foam. FABMS: 258.2

Step D: N-Benzyloxycarbonyl-Pro-N-t-Boc-HCAP ester (19-4)

A solution of 1.62 g (6.6 mmol) of N-Z-Pro-OH, 1.54 g (6.0 mmol) of N-Boc-HCAP (19-3), 1.26 g (6.6 mmol) of EDC, and 146 mg (1.2 mmol) of DMAP in 30 mL of anhydrous CH₂Cl₂ was reated and the resulting solution stirred at room temp. in an N₂ atmosphere for 12 h. The solvent was removed in vacuo, the residue dissolved in ethyl acetate (150 ml) and the solution extracted with 0.5 N NaHCO₃ (50 ml), water (50 ml) and brine, then dried and concentrated to provide the crude coupling product.

Step E: H-Pro-N-t-Boc-HCAP ester (19-5)

A 2.0 g of (19-4) in a solution of 90 mL EtOH, 20 ml water, and 10 mL acetic acid was hydrogenated on a Parr apparatus at 50 psi over 200 mg of Pd(OH)2 catalyst for 3 h. The reaction was filtered through a Celite pad, and the filtrate was concentrated to small volume in vacuo, then purified by preparatory HPLC on a Delta-Pak C18 column with 0.1% trifluoroacetic acid-aqueous acetonitrile solvent systems using 95-50% A, 60 min linear gradient. Fractions containing product of at least 99% (HPLC) purity were combined to give the title compound (19-5). FABMS: 356.3

Step F: N-Acetyl -Ser-Chg-Gln-Ser-Ser-Pro-HCAP amine (19-6)

A solution of 589 mg (1.0 mmol) of N-Acetyl-Ser-Chg-Gln-Ser-Ser-OH (19-1), 356 mg (1.0 mmol) of H-Pro-N-t-Boc-HCAP ester (19-5), 229 mg (1.2 mmol) of EDC, and 81 mg (0.5 mmol) of ODBHT (3,4-dihydro-3-hydroxy-4-oxo-1,2,3-benzotriazine), in 7 mL of DMF was stirred at 0° C. in an N₂ atmosphere for 10 h. The solvent was removed in vacuo, the residue was washed with ether and dried. The crude product was treated with 95% TFA :5% H₂O (20 ml) for 2 hr at r.t. under argon. After evaporation of the TFA, the residue was purified by preparatory HPLC on a Delta-Pak C18 column with 0.1% trifluoroacetic acid-aqueous acetonitrile solvent systems using 95-50% A, 60 min linear gradient. Fractions containing product of at least 99% (HPLC) purity were combined to give the title compound (19-6).

FABMS: 825.5

Peptide Content: 893.6 NMole/mg.

HPLC: 99% pure @214 nm, retention time=15.2 min, (Vydac C₁₈, gradient of 95% A/B to 5% A/B over 30 min, A=0.1% TFA-H₂O, B=0.1% TFA-CH₃CN)

Step G: 4-des-Acetylvinblastine-23-(N-Ac-Ser-Chg-Gln-Ser-Ser-Pro-HCAP) amide acetate salt (19-7)

A solution of 0.461 of 4-des-acetylvinblastine-23-hydrazide (0.6 mmol) in 10 mL DMF cooled to −15° C. under Argon, was converted to the azide in situ by acidification with 4M HCl in dioxane to pH<1.5 (moistened 0-2.5 range paper), followed by addition of 0.105 mL (1.3 equiv) of isoamyl nitrite and stirring for 1 hr at 10-15° C. The pH was brought to 7 by the addition of DIEA, and 545 mg (0.66 mmol) of amine derivative (19-6) from step F was then added, and the reaction was stirred at 0° C. for 24 hrs, and purified by preparatory HPLC on a 15μM,100A, Delta-Pak C18 column with 0.1% trifluoroacetic acid-aqueous acetonitrile solvent systems using 95-50% A, 60 min linear gradient. Homogeneous fractions were pooled and concentrated in vacuo, followed by freeze-drying to give the title compound as the TFA salt which was converted to title compound by AG 4×4 resin (100-200 mesh, free base form, BIO-RAD) (19-7)

ES⁺:1560.9

Peptide Content: 586.8 NMole/mg.

Ser 3.04; Chg 1.01; Glu 1.00; Pro 0.97

HPLC: 99% pure @214 nm, retention time=13.4 min, (Vydac C₁₈, gradient of 95% A/B to 5% A/B over 30 min, A=0.1% TFA-H₂O, B=0.1% TFA-CH₃CN)

Biological Assays

The ability of the compounds useful in the methods of the present invention to inhibit angiogenesis can be demonstrated using the following assays.

### Angiogenesis Inhibitor Assays

The compounds of the instant invention described in the Examples were tested by the assays described below and were found to have kinase inhibitory activity. Other assays are known in the literature and could be readily performed by those of skill in the art. (see, for example, Dhanabal et al., Cancer Res. 59:189-197; Xin et al., J. Biol. Chem. 274:9116-9121; Sheu et al., Anticancer Res. 18:4435-4441; Ausprunk et al., Dev. Biol. 38:237-248; Gimbrone et al., J. Natl. Cancer Inst. 52:413-427; Nicosia et al., In Vitro 18:538-549).

VEGF Receptor Kinase Assay

VEGF receptor kinase activity is measured by incorporation of radio-labeled phosphate into polyglutamic acid, tyrosine, 4:1 (pEY) substrate. The phosphorylated pEY product is trapped onto a filter membrane and the incorporation of radio-labeled phosphate quantified by scintillation counting.

### Materials

VEGF Receptor Kinase

The intracellular tyrosine kinase domains of human KDR (Terman, B. I. et al. Oncogene (1991) vol. 6, pp. 1677-1683.) and Flt-1 (Shibuya, M. et al. Oncogene (1990) vol. 5, pp. 519-524) were cloned as glutathione S-transferase (GST) gene fusion proteins. This was accomplished by cloning the cytoplasmic domain of the KDR kinase as an in frame fusion at the carboxy terminus of the GST gene. Soluble recombinant GST-kinase domain fusion proteins were expressed in Spodoptera frugiperda (Sf21) insect cells (Invitrogen) using a baculovirus expression vector (pAcG2T, Pharmingen).

Lysis Buffer

50 mM Tris pH 7.4, 0.5 M NaCl, 5 mM DTT, 1 mM EDTA, 0.5% triton X-100, 10% glycerol, 10 mg/mL of each leupeptin, pepstatin and aprotinin and 1 mM phenylmethylsulfonyl fluoride (all Sigma).

Wash Buffer

50 mM Tris pH 7.4, 0.5 M NaCl, 5 mM DTT, 1 mM EDTA, 0.05% triton X-100, 10% glycerol, 10 mg/mL of each leupeptin, pepstatin and aprotinin and 1 mM phenylmethylsulfonyl fluoride.

Dialysis Buffer

50 mM Tris pH 7.4,0.5 M NaCl, 5 mM DTT, 1 mM EDTA, 0.05% triton X-100, 50% glycerol, 10 mg/mL of each leupeptin, pepstatin and aprotinin and 1 mM phenylmethylsuflonyl fluoride.

10× Reaction Buffer

200 mM Tris, pH 7.4, 1.0 M NaCl, 50 mM MnCl_{2, 10} mM DTT and 5 mg/mL bovine serum albumin (Sigma).

Enzyme Dilution Buffer

50 mM Tris, pH 7.4, 0.1 M NaCl, 1 mM DTT, 10% glycerol, 100 mg/mL BSA.

10× Substrate

750 μg/mL poly (glutamic acid, tyrosine; 4:1) (Sigma).

Stop Solution

30% trichloroacetic acid, 0.2 M sodium pyrophosphate (both Fisher).

Wash Solution

15% trichloroacetic acid, 0.2 M sodium pyrophosphate.

Filter Plates

Millipore #MAFC NOB, GF/C glass fiber 96 well plate.

Method A-Protein Purification

1. Sf21 cells were infected with recombinant virus at a multiplicity of infection of 5 virus particles/cell and grown at 27° C. for 48 hours.

2. All steps were performed at 4° C. Infected cells were harvested by centrifugation at 1000×g and lysed at 4° C. for 30 minutes with 1/10 volume of lysis buffer followed by centrifugation at 100,000×g for 1 hour. The supernatant was then passed over a glutathione Sepharose column (Pharmacia) equilibrated in lysis buffer and washed with 5 volumes of the same buffer followed by 5 volumes of wash buffer. Recombinant GST-KDR protein was eluted with wash buffer/10 mM reduced glutathione (Sigma) and dialyzed against dialysis buffer.

Method B-VEGF Receptor Kinase Assay

1. Add 5 μl of inhibitor or control to the assay in 50% DMSO.

2. Add 35 μl of reaction mix containing 5 μl of 10× reaction buffer, 5 μl 25 mM ATP/10 μCi [³³P]ATP (Amersham), and 5 μl 10× substrate.

3. Start the reaction by the addition of 10 μl of KDR (25 nM) in enzyme dilution buffer.

4. Mix and incubate at room temperature for 15 minutes.

5. Stop by the addition of 50 μl stop solution.

6. Incubate for 15 minutes at 4° C.

7. Transfer a 90 μl aliquot to filter plate.

8. Aspirate and wash 3 times with wash solution.

9. Add 30 μl of scintillation cocktail, seal plate and count in a Wallac Microbeta scintillation counter.

Human Umbilical Vein Endothelial Cell Mitogenesis Assay

Expression of VEGF receptors that mediate mitogenic responses to the growth factor is largely restricted to vascular endothelial cells. Human umbilical vein endothelial cells (HUVECs) in culture proliferate in response to VEGF treatment and can be used as an assay system to quantify the effects of KDR kinase inhibitors on VEGF stimulation. In the assay described, quiescent HUVEC monolayers are treated with vehicle or test compound 2 hours prior to addition of VEGF or basic fibroblast growth factor (bFGF). The mitogenic response to VEGF or bFGF is determined by measuring the incorporation of [³H]thymidine into cellular DNA.

### Materials

HUVECs

HUVECs frozen as primary culture isolates are obtained from Clonetics Corp. Cells are maintained in Endothelial Growth Medium (EGM; Clonetics) and are used for mitogenic assays at passages 3-7.

Culture Plates

NUNCLON 96-well polystyrene tissue culture plates (NUNC #167008).

Assay Medium

Dulbecco's modification of Eagle's medium containing 1 g/mL glucose (low-glucose DMEM; Mediatech) plus 10% (v/v) fetal bovine serum (Clonetics).

Test Compounds

Working stocks of test compounds are diluted serially in 100% dimethylsulfoxide (DMSO) to 400-fold greater than their desired final concentrations. Final dilutions to 1× concentration are made directly into Assay Medium immediately prior to addition to cells.

10× Growth factors

Solutions of human VEGF₁₆₅ (500 ng/mL; R&D Systems) and bFGF (10 ng/mL; R&D Systems) are prepared in Assay Medium.

10 ×[³H]Thymidine

[Methyl-³H]Thymidine (20 Ci/mmol; Dupont-NEN) is diluted to 80 uCi/mL in low-glucose DMEM.

Cell Wash Medium

Hank's balanced salt solution (Mediatech) containing 1 mg/mL bovine serum albumin (Boehringer-Mannheim).

Cell Lysis Solution

1 N NaOH, 2% (w/v) Na2CO₃.

Method 1

HUVEC monolayers maintained in EGM are harvested by trypsinization and plated at a density of 4000 cells per 100 μL Assay Medium per well in 96-well plates. Cells are growth-arrested for 24 hours at 37° C. in a humidified atmosphere containing 5% C₀₂.

Method 2

Growth-arrest medium is replaced by 100 μL Assay Medium containing either vehicle (0.25% [v/v]DMSO) or the desired final concentration of test compound. All determinations are performed in triplicate. Cells are then incubated at 37° C./5% CO₂ for 2 hours to allow test compounds to enter cells.

Method 3

After the 2-hour pretreatment period, cells are stimulated by addition of 10 μL/well of either Assay Medium, 10× VEGF solution or 10× bFGF solution. Cells are then incubated at 37° C./5% CO₂.

Method 4

After 24 hours in the presence of growth factors, 10× [³H]Thymidine (10 μL/well) is added.

Method 5

Three days after addition of [³H]thymidine, medium is removed by aspiration, and cells are washed twice with Cell Wash Medium (400 μL/well followed by 200 μL/well). The washed, adherent cells are then solubilized by addition of Cell Lysis Solution (100 μL/well) and warming to 37° C. for 30 minutes. Cell lysates are transferred to 7-mL glass scintillation vials containing 150 μL of water. Scintillation cocktail (5 mL/vial) is added, and cell-associated radioactivity is determined by liquid scintillation spectroscopy.

Based upon the foregoing assays the compounds of formula I are inhibitors of VEGF and thus are useful for the inhibition of angiogenesis, such as in the treatment of ocular disease, e.g., diabetic retinopathy and in the treatment of cancers, e.g., solid tumors. The instant compounds inhibit VEGF-stimulated mitogenesis of human vascular endothelial cells in culture with IC₅₀ values between 0.01-5.0 μM. These compounds also show selectivity over related tyrosine kinases (e.g., FGFR1 and the Src family; for relationship between Src kinases and VEGFR kinases, see Eliceiri et al., Molecular Cell, Vol. 4, pp.915-924, December 1999).

### PSA Conjugate Assays

Assessment of the Recognition of Oligopeptide-Cytotoxic Drug Conjugates by Free PSA

The PSA conjugates, prepared as described above and in particular in Examples 11-19, are individually dissolved in PSA digestion buffer (50 mM tris(hydroxymethyl)-aminomethane pH7.4, 140 mM NaCl) and the solution added to PSA at a molar ration of 100 to 1. Alternatively, the PSA digestion buffer utilized is 50 mM tris(hydroxymethyl)-aminomethane pH7.4, 140 mM NaCl. The reaction is quenched after various reaction times by the addition of trifluoroacetic acid (TFA) to a final 1% (volume/volume). Alternatively the reaction is quenched with 10 mM ZnCl₂. The quenched reaction is analyzed by HPLC on a reversed-phase C18 column using an aqueous 0.1% TFA/acetonitrile gradient. The amount of time (in minutes) required for 50% cleavage of the noted oligopeptide-cytotoxic agent conjugates with enzymatically active free PSA were then calculated.

In vitro Assay of Cytotoxicity of Peptidyl Derivatives of Doxorubicin

The cytotoxicities of the cleaveable oligopeptide-doxorubicin conjugates, prepared as described above and in particular in Examples 11-19, against a line of cells which is known to be killed by unmodified doxorubicin are assessed with an Alamar Blue assay. Specifically, cell cultures of LNCap prostate tumor cells (which express enzymatically active PSA) or DuPRO cells in 96 well plates are diluted with medium (Dulbecco's Minimum Essential Medium-α[MEM-α]) containing various concentrations of a given conjugate (final plate well volume of 200 μl). The cells are incubated for 3 days at 37° C., 20 μl of Alamar Blue is added to the assay well. The cells are further incubated and the assay plates are read on a EL-310 ELISA reader at the dual wavelengths of 570 and 600 nm at 4 and 7 hours after addition of Alamar Blue. Relative percentage viability at the various concentration of conjugate tested is then calculated versus control (no conjugate) cultures.

In vitro Assay of Cytotoxicity of Peptidyl Derivatives of Vinca Drugs

The cytotoxicities of the cleaveable oligopeptide-cytotoxic drug conjugates, prepared as described above and in particular in Examples 11-19, against a line of cells which is known to be killed by unmodified vinca drug was assessed with an Alamar Blue assay. Specifically, cell cultures of LNCap prostate tumor cells, Colo320DM cells (designated C320) or T47D cells in 96 well plates are diluted with medium containing various concentrations of a given conjugate (final plate well volume of 200 μl). The Colo320DM cells, which do not express free PSA, are used as a control cell line to determine non-mechanism based toxicity. The cells are incubated for 3 days at 37° C., 20 μl of Alamar Blue is added to the assay well. The cells are further incubated and the assay plates are read on a EL-310 ELISA reader at the dual wavelengths of 570 and 600 nm at 4 and 7 hours after addition of Alamar Blue. Relative percentage viability at the various concentration of conjugate tested is then calculated versus control (no conjugate) cultures and an EC₅₀ was determined.

In vivo Efficacy of Peptidyl-Cytotoxic Agent Conjugates

LNCaP.FGC or DuPRO-1 cells are trypsinized, resuspended in the growth medium and centifuged for 6 mins. at 200×g. The cells are resuspended in serum-free MEM-α and counted. The appropriate volume of this solution containing the desired number of cells is then transferred to a conical centrifuge tube, centrifuged as before and resuspended in the appropriate volume of a cold 1:1 mixture of MEM-α-Matrigel. The suspension is kept on ice until the animals are inoculated.

Harlan Sprague Dawley male nude mice (10-12 weeks old) are restrained without anesthesia and are inoculated with 0.5 mL of cell suspension on the left flank by subcutaneous injection using a 22 G needle. Mice are either given approximately 5×10⁵ DuPRO cells or 1.5×10⁷ LNCaP.FGC cells.

Following inoculation with the tumor cells the mice are treated under one of two protocols:

Protocol A

One day after cell inoculation the animals are dosed with a 0.1-0.5 mL volume of test conjugate, vinca drug or vehicle control (sterile water). Dosages of the conjugate and vinca drug are initially the maximum non-lethal amount, but may be subsequently titrated lower. Identical doses are administered at 24 hour intervals for 5 days. After 10 days, blood samples are removed from the mice and the serum level of PSA is determined. Similar serum PSA levels are determined at 5-10 day intervals. At the end of 5.5 weeks the mice are sacrificed and weights of any tumors present are measured and serum PSA again determined. The animals' weights are determined at the beginning and end of the assay.

Protocol B

Ten days after cell inoculation, blood samples are removed from the animals and serum levels of PSA are determined. Animals are then grouped according to their PSA serum levels. At 14-15 days after cell inoculation, the animals are dosed with a 0.1-0.5 mL volume of test conjugate, vinca drug or vehicle control (sterile water). Dosages of the conjugate and vinca drug are initially the maximum non-lethal amount, but may be subsequently titrated lower. Identical doses are administered at 24 hour intervals for 5 days. Serum PSA levels are determined at 5-10 day intervals. At the end of 5.5 weeks the mice are sacrificed, weights of any tumors present are measured and serum PSA again determined. The animals' weights are determined at the beginning and end of the assay.

In vivo Efficacy of Administration of a Combination of a PSA Conjugate and an Inhibitor of Angiogenesis

Male nude mice (4 groups of 15) are injected subcutaneously with 1.5×10⁷ LNCaP.FGC cells (available from the American Type Culture Collection, ATCC No. CRL-1740; see also J. S. Horoszewicz et al. Cancer Res., 43:1809-1818 (1983)) in 80% Matrigel.

Beginning five days after the tumor cell implantation, a test angiogenesis inhibitor is administered by oral gavage. The concentration of the test angiogenesis inhibitor is adjusted to provide a therapeutically minimal or subminimal plasma concentration of the inhibitor of angiogenesis. For example, if the compound of Example 4 is being tested in combination with a PSA conjugate, the concentration of the compound in the food is adjusted so that a continuous plasma concentration of between 5-20 μM is maintained. Administration of between 1.0 and 100 mpk of an angiogenesis inhibitor compound such as is described in Examples 3-10 is expected to produce the preferred plasma concentrations.

Administration of the inhibitor of angiogenesis is as follows:

Group A: Administration of test inhibitor of angiogenesis compound

Group B: Administration of vehicle.

Group C: Administration of test inhibitor of angiogenesis compound

Group D: Administration of vehicle

Beginning at the same time as administration of the inhibitor of angiogenesis, a solution of test PSA conjugate is administered to Groups A and B. Vehicle is administered to Groups C and D. The PSA conjugate is administered IV as a therapeutically minimal dose. For example, when the PSA conjugate described in Example 14 is tested, a 0.20 mL of a solution of test PSA conjugate, (3-5 mpk, 34.1 mL D5W+80 μL 7.5% sodium bicarbonate) is administered to Groups A and B. Vehicle (0.20 mL) is administered to Groups C and D.

Three days after the initial dosing of the inhibitor of angiogenesis and the PSA conjugate, three mice from each group are bled from the tail vein to assess serum levels of the test inhibitor of angiogenesis.

After the initial dose of PSA conjugate, the animals are administered PSA conjugate solution either as four additional doses (one/day) of the test PSA conjugate solution or vehicle are administered to the respective Groups over four consecutive days, or once a week for four consecutive weeks.

At the end of 5-6 weeks after the innoculation with the LNCaP cells, the mice are bled from the tail vein and the plasma PSA level is measured using a Tandem®-E PSA ImmunoEnzyMetri Assay kit (Hybritech). The plasma concentration of the inhibitor of angiogenesis is also determined at this time. The mice are then sacrificed, weighed, tumors excised and weighed.

In Vitro Determination of Proteolytic Cleavage of Conjugates by Endogenous Non-PSA Proteases

Step A: Preparation of Proteolytic Tissue Extracts

All procedures are carried out at 4° C. Appropriate animals are sacrificed and the relevant tissues are isolated and stored in liquid nitrogen. The frozen tissue is pulverized using a mortar and pestle and the pulverized tissue is transfered to a Potter-Elvejeh homogenizer and 2 volumes of Buffer A (50 mM Tris containing 1.15% KCl, pH 7.5) are added. The tissue is then disrupted with 20 strokes using first a loose fitting and then a tight fitting pestle. The homogenate is centrifuged at 10,000×g in a swinging bucket rotor (HB4-5), the pellet is discarded and the re-supernatant centrifuged at 100,000×g (Ti 70). The supernatant (cytosol) is saved.

The pellet is resuspended in Buffer B (10 mM EDTA containing 1.15% KCl, pH 7.5) using the same volume used in step as used above with Buffer A. The suspension is homogenized in a dounce homogenizer and the solution centrifuged at 100,000×g. The supernatant is discarded and the pellet resuspended in Buffer C(10 mM potassium phosphate buffer containing 0.25 M sucrose, pH 7.4), using ½ the volume used above, and homogenized with a dounce homogenizer.

Protein content of the two solutions (cytosol and membrane) is determined using the Bradford assay. Assay aliquots are then removed and frozen in liquid N₂. The aliquots are stored at −70° C.

Step B: Proteolytic Cleavage Assay

For each time point, 20 microgram of a test PSA conjugate and 150 micrograms of tissue protein, prepared as described in Step A and as determined by Bradford in reaction buffer are placed in solution of final volume of 200 microliters in buffer (50 mM TRIS, 140 mM NaCl, pH 7.2). Assay reactions are run for 0, 30, 60, 120, and 180 minutes and are then quenched with 9 microliters of 0.1 M ZnCl₂ and immediately placed in boiling water for 90 seconds. Reaction products are analyzed by HPLC using a VYDAC C18 15 cm column in water/acetonitrile (5% to 50% acetonitrile over 30 minutes).

What is claimed is:

1. A method for treating cancer in a mammal in need thereof which comprises administering to said mammal amounts of at least one inhibitor of angiogenesis and at least one PSA conjugate.

2. The method according to claim 1 wherein an amount of an inhibitor of angiogenesis and an amount of an PSA conjugate are administered consecutively.

3. The method according to claim 1 wherein an amount of an inhibitor of angiogenesis and an amount of an PSA conjugate are administered simultaneously.

4. The method according to claim 1 wherein the therapeutic effect is selected from inhibition of cancerous tumor growth and regression of cancerous tumors.

5. The method according to claim 1 wherein the cancer is a cancer related to cells that express enzymatically active PSA.

6. The method according to claim 1 wherein the cancer is prostate cancer.

7. The method according to claim 1 wherein the PSA conjugate is selected from:
a) a compound represented by the formula IX: 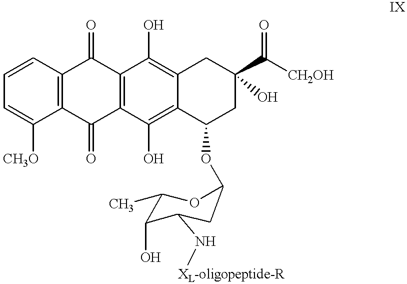
wherein:
oligopeptide is an oligopeptide which is selectively recognized by the free prostate specific antigen (PSA) and is capable of being proteolytically cleaved by the enzymatic activity of the free prostate specific antigen;
X_{L} is absent or is an amino acid selected from:
a) phenylalanine,
b) leucine,
c) valine,
d) isoleucine,
e) (2-naphthyl)alanine,
f) cyclohexylalanine,
g) diphenylalanine,
h) norvaline, and
j) norleucine;
R is hydrogen or —(C═O)R¹; and
R¹ is C₁-C₆-alkyl or aryl,
or the pharmaceutically acceptable salt thereof;
b) a compound represented by the formula X: 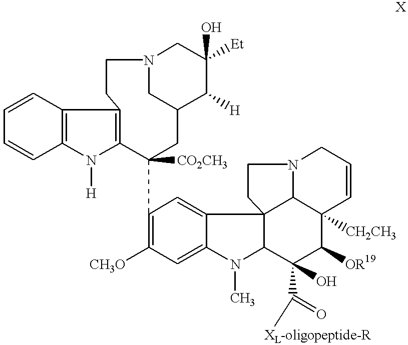
wherein:
oligopeptide is an oligopeptide which is selectively recognized by the free prostate specific antigen (PSA) and is capable of being proteolytically cleaved by the enzymatic activity of the free prostate specific antigen;
X_{L} is absent or is an amino acid selected from:
a) phenylalanine,
b) leucine,
c) valine,
d) isoleucine,
e) (2-naphthyl)alanine,
f) cyclohexylalanine,
g) diphenylalanine,
h) norvaline, and
j) norleucine; or
XL is —NH—(CH₂)ₙ—NH—
R is hydrogen or —(C═O)R¹;
R¹ is C₁-C₆-alkyl or aryl;
R¹⁹ is hydrogen or acetyl; and
n is 1, 2, 3, 4 or 5,
or the pharmaceutically acceptable salt thereof;
c) a compound represented by the formula XI: 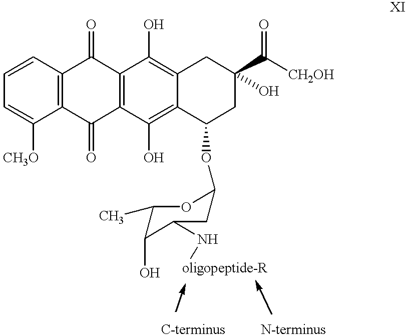
wherein:
oligopeptide is an oligopeptide which is selectively recognized by the free prostate specific antigen (PSA) and is capable of being proteolytically cleaved by the enzymatic activity of the free prostate specific antigen, wherein the oligopeptide comprises a cyclic amino acid of the formula: 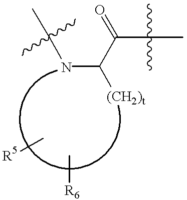
and wherein the C-terminus carbonyl is covalently bound to the amine of doxorubicin;
R is selected from
a) hydrogen,
b) —(C═O)R^{1a}, 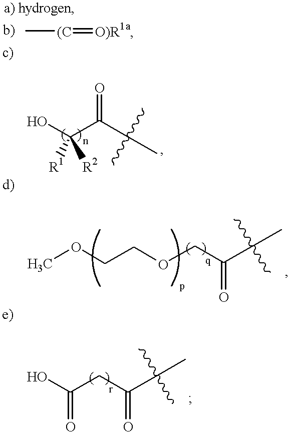
R¹ and R² are independently selected from: hydrogen, OH, C₁-C₆ alkyl, C₁-C₆ alkoxy, C₁-C₆ aralkyl and aryl;
R^{1a} is C₁-C₆-alkyl, hydroxylated aryl, polyhydroxylated aryl or aryl;
R⁵ is selected from HO— and C₁-C₆ alkoxy;
R⁶ is selected from hydrogen, halogen, C₁-C₆ alkyl, HO— and C₁-C₆ alkoxy; and
n is 1, 2, 3 or 4;
p is zero or an integer between 1 and 100;
q is 0 or 1, provided that if p is zero, q is 1;
r is an integer between 1 and 10; and
t is 3 or 4;
or a pharmaceutically acceptable salt thereof;
d) a compound represented by the formula X: 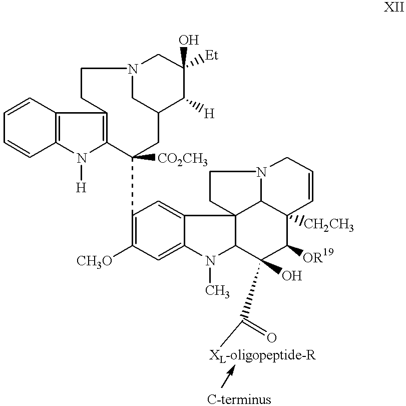
wherein:
oligopeptide is an oligopeptide which is selectively recognized by the free prostate specific antigen (PSA) and is capable of being proteolytically cleaved by the enzymatic activity of the free prostate specific antigen, and the oligopeptide comprises a cyclic amino acid of the formula: 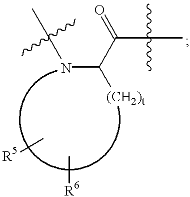
X_{L} is —NH—(CH₂)ᵤ—NH—
R is selected from
a) hydrogen,
b) —(C═O)R^{1a}, 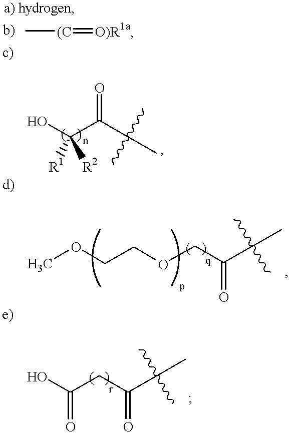
R¹ and R² are independently selected from: hydrogen, OH, C₁-C₆ alkyl, C₁-C₆ alkoxy, C₁-C₆ aralkyl and aryl;
R^{1a} is C₁-C₆-alkyl, hydroxylated aryl, polyhydroxylated aryl or aryl,
R¹⁹ is hydrogen, (C₁-C₃ alkyl)-CO, or chlorosubstituted (C₁-C₃ alkyl)-CO;
n is 1, 2, 3 or 4;
p is zero or an integer between 1 and 100;
q is 0 or 1, provided that if p is zero, q is 1;
r is 1, 2 or 3;
t is 3 or 4;
u is 1, 2, 3, 4 or 5,
or the pharmaceutically acceptable salt thereof;
e) a compound represented by the formula XI: 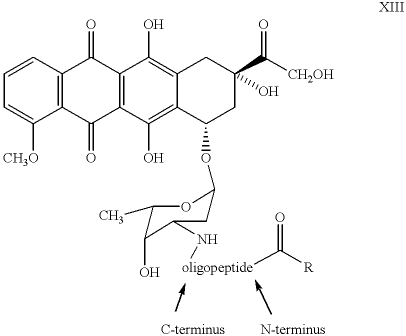
wherein:
oligopeptide is an oligopeptide which is selectively recognized by the free prostate specific antigen (PSA) and is capable of being proteolytically cleaved by the enzymatic activity of the free prostate specific antigen, and wherein the C-terminus carbonyl is covalently bound to the amine of doxorubicin and the N-terminus amine is covalently bound to the carbonyl of the blocking group;
R is selected from 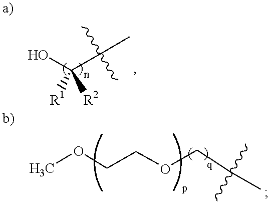
R¹ and R² are independently selected from: hydrogen, OH, C₁-C₆ alkyl, C₁-C₆ alkoxy, C₁-C₆ aralkyl and aryl;
n is 1, 2, 3 or 4;
p is zero or an integer between 1 and 100;
q is 0 or 1, provided that if p is zero, q is 1;
or the pharmaceutically acceptable salt thereof;
f) a compound represented by the formula XIV: 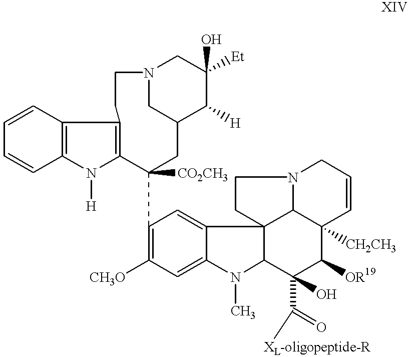
wherein:
oligopeptide is an oligopeptide which is selectively recognized by the free prostate specific antigen (PSA) and is capable of being proteolytically cleaved by the enzymatic activity of the free prostate specific antigen;
X_{L} is —NH—(CH₂)ᵣ—NH—
R is selected from 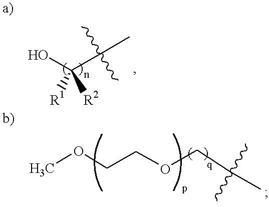
R¹ and R² are independently selected from: hydrogen, OH, C₁-C₆ alkyl, C₁-C₆ alkoxy, C₁-C₆ aralkyl and aryl;
R¹⁹ is hydrogen, (C₁-C₃ alkyl)-CO, or chlorosubstituted (C₁-C₃ alkyl)-CO;
n is 1, 2, 3 or 4;
p is zero or an integer between 1 and 100;
q is 0 or 1, provided that if p is zero, q is 1;
r is 1, 2, 3, 4 or 5,
or the pharmaceutically acceptable salt thereof;
g) a compound represented by the formula XV: 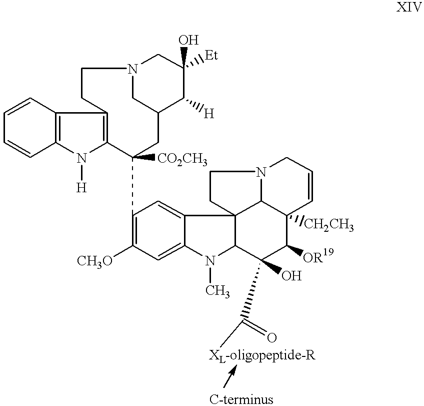
wherein:
oligopeptide is an oligopeptide which is selectively recognized by the free prostate specific antigen (PSA) and is capable of being proteolytically cleaved by the enzymatic activity of the free prostate specific antigen,
X_{L} is —NH—(CH₂)ᵤ—W—(CH₂)ᵤ—NH—
R is selected from
a) hydrogen,
b) —(C═O)R^{1a}, 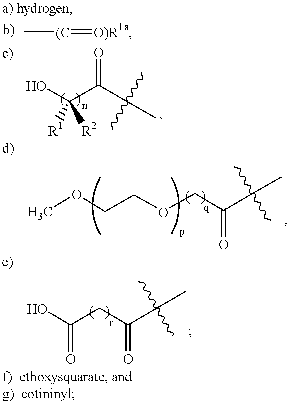
f) ethoxysquarate, and
g) cotininyl;
R¹ and R² are independently selected from: hydrogen, OH, C₁-C₆ alkyl, C₁-C₆ alkoxy, C₁-C₆ aralkyl and aryl;
R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl;
R⁹ is hydrogen, (C₁-C₃ alkyl)-CO, or chlorosubstituted (C₁-C₃ alkyl)-CO;
W is selected from cyclopentyl, cyclohexyl, cycloheptyl or bicyclo[2.2.2]octanyl;
n is 1, 2, 3 or 4;
p is zero or an integer between 1 and 100;
q is 0 or 1, provided that if p is zero, q is 1;
r is 1, 2 or 3;
t is 3 or 4;
u is 0, 1, 2 or 3,
or the pharmaceutically acceptable salt thereof; and
h) a compound represented by the formula XVI: 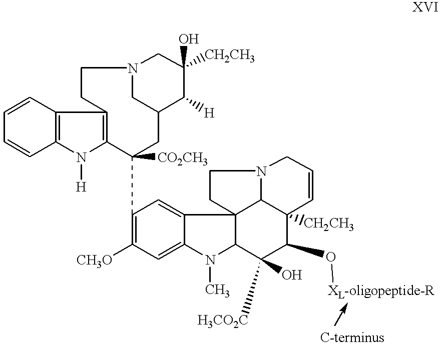
wherein:
oligopeptide is an oligopeptide which is selectively recognized by the free prostate specific antigen (PSA) and is capable of being proteolytically cleaved by the enzymatic activity of the free prostate specific antigen,
X_{L} is selected from: a bond, —C(O)—(CH₂)ᵤ—W—(CH₂)ᵤ—O— and —C(O)—(CH₂)ᵤ—W—(CH₂)ᵤ—NH—;
R is selected from
a) hydrogen,
b) —(C═O)R^{1a}, 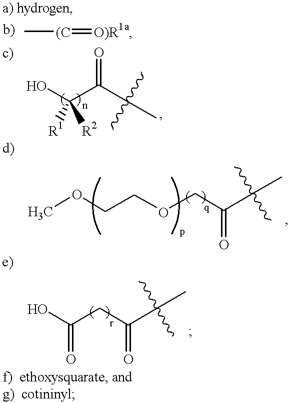
f) ethoxysquarate, and
g) cotininyl;
R¹ and R² are independently selected from: hydrogen, OH, C₁-C₆ alkyl, C₁-C₆ alkoxy, C₁-C₆ aralkyl and aryl;
R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl;
R⁹ is hydrogen, (C₁-C₃ alkyl)-CO, or chlorosubstituted (C₁-C₃ alkyl)-CO;
W is selected from a branched or straight chain C₁-C₆-alkyl, cyclopentyl, cyclohexyl, cycloheptyl or bicyclo[2.2.2]octanyl;
n is 1, 2, 3 or 4;
p is zero or an integer between 1 and 100;
q is 0 or 1, provided that if p is zero, q is 1;
r is 1, 2 or 3;
t is 3 or 4;
u is 0, 1, 2 or 3;
or the pharmaceutically acceptable salt or optical isomer thereof.

8. The method according to claim 7 wherein the PSA conjugate is selected from: 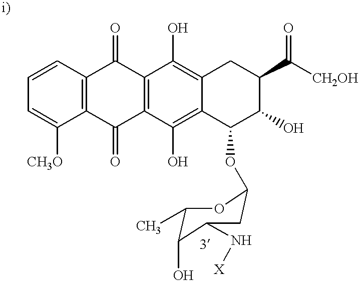
wherein X is:
AsnLysIleSerTyrGlnSer—(SEQ.ID.NO.: 1),
AsnLysIleSerTyrGlnSerSer—(SEQ.ID.NO.: 2),
AsnLysIleSerTyrGlnSerSerSer—(SEQ.ID.NO.:3),
AsnLysIleSerTyrGlnSerSerSerThr—(SEQ.ID.NO.:4),
AsnLysIleSerTyrGlnSerSerSerThrGlu—(SEQ.ID.NO.: 5),
AlaAsnLysIleSerTyrGlnSerSerSerThrGlu—(SEQ.ID.NO.: 6),
Ac—AlaAsnLysIleSerTyrGlnSerSerSerThr—(SEQ.ID.NO.: 7),
Ac—AlaAsnLysIleSerTyrGlnSerSerSerThrLeu—(SEQ.ID.NO.: 8),
Ac—AlaAsnLysAlaSerTyrGlnSerAlaSerThrLeu—(SEQ.ID.NO.: 9),
Ac—AlaAsnLysAlaSerTyrGlnSerAlaSerLeu—(SEQ.ID.NO.: 10),
Ac—AlaAsnLysAlaSerTyrGlnSerSerSerLeu—(SEQ.ID.NO.: 11),
Ac—AlaAsnLysAlaSerTyrGlnSerSerLeu—(SEQ.ID.NO.: 12),
Ac—SerTyrGlnSerSerSerLeu—(SEQ.ID.NO.: 13),
Ac—hArgTyrGlnSerSerSerLeu—(SEQ.ID.NO.: 14).
Ac—LysTyrGlnSerSerSerLeu—(SEQ.ID.NO.: 15),
Ac—LysTyrGlnSerSerNle—(SEQ.ID.NO.: 16), 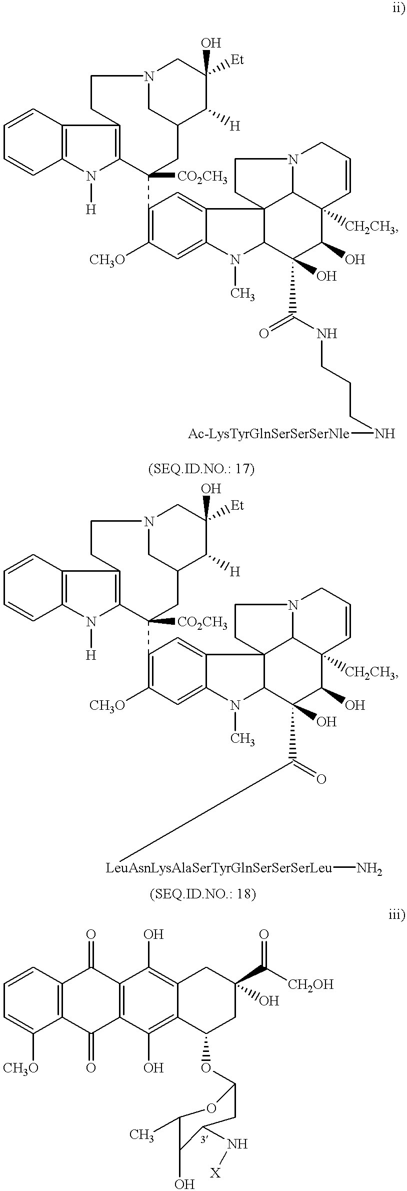
wherein X is: 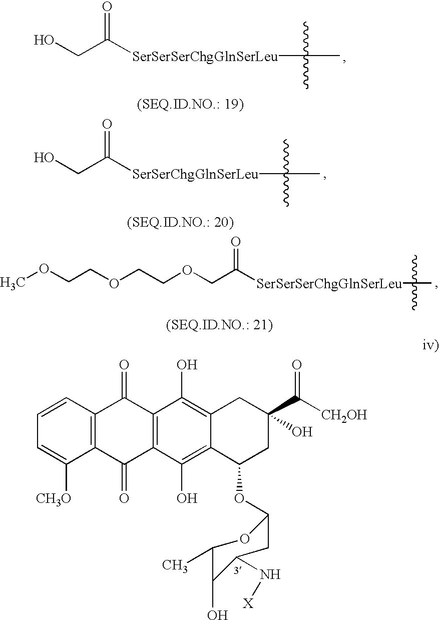
wherein X is: 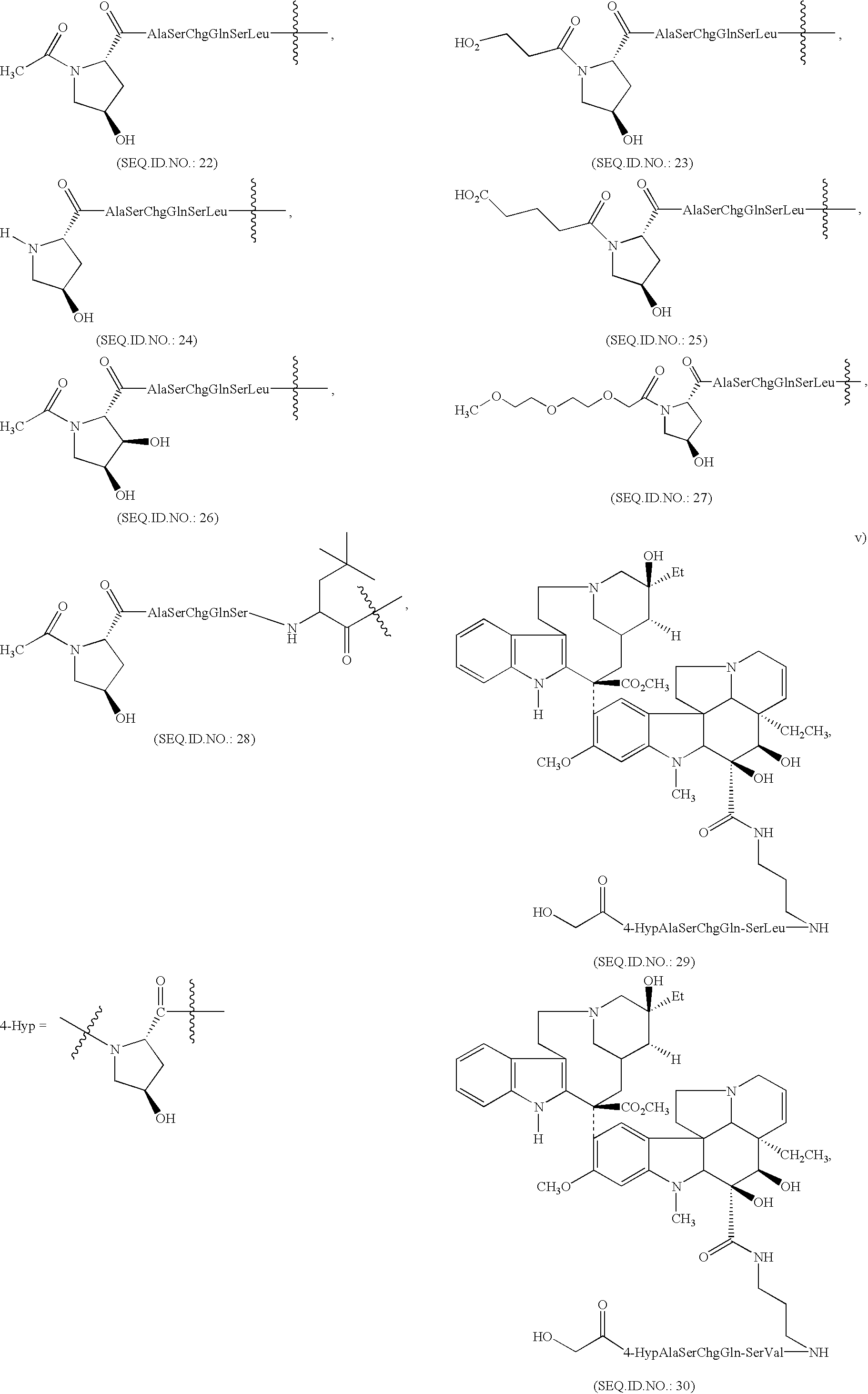 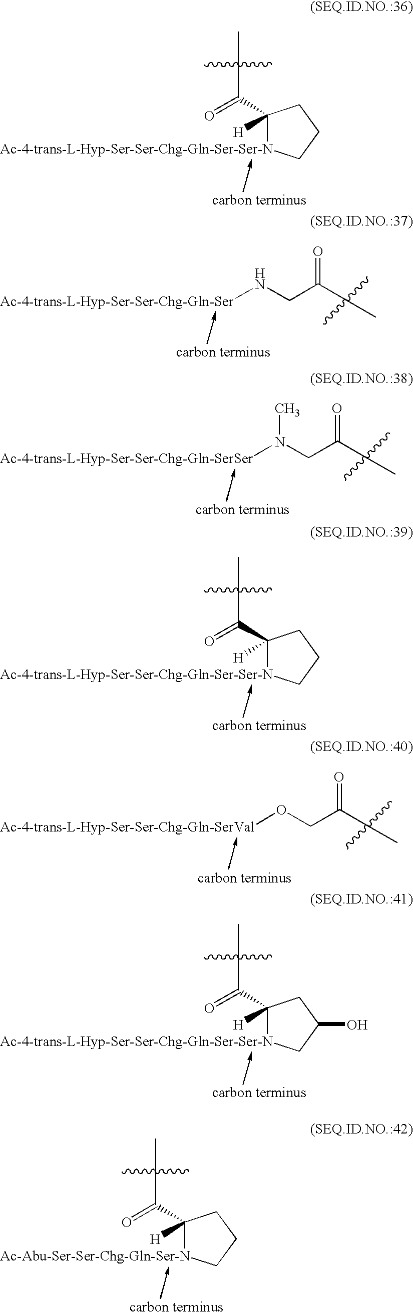
or a pharmaceutically acceptable salt or optical isomer thereof.

9. The method according to claim 7 wherein the PSA conjugate is: 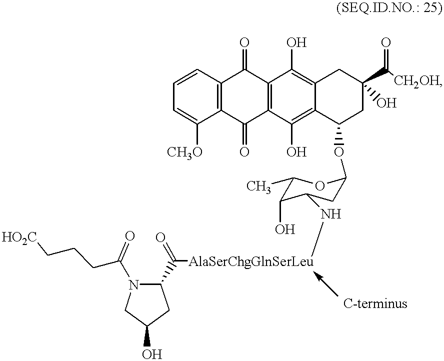
or a pharmaceutically acceptable salt thereof.

10. The method according to claim 1 wherein the inhibitor of angiogenesis is selected from an inhibitor of matrix metalloproteinases, an inhibitor of the growth of endothelial cells, an inhibitor of endothelial-specific integrin/survival signaling, and a compound that blocks the activators of angiogenesis factors.

11. The method according to claim 10 wherein the inhibitor of angiogenesis is an inhibitor of matrix metalloproteinases.

12. The method according to claim 10 wherein the inhibitor of angiogenesis is an inhibitor of the growth of endothelial cells.

13. The method according to claim 10 wherein the inhibitor of angiogenesis is an inhibitor of endothelial specific integrin/survival signaling.

14. The method according to claim 10 wherein the inhibitor of angiogenesis is a compound that blocks the activators of angiogenesis factors.

15. The method according to claim 14 wherein the inhibitor of angiogenesis is an inhibitor of KDR.

16. The method according to claim 15 wherein the inhibitor of KDR is selected from:
(a) a compound represented by formula (I): 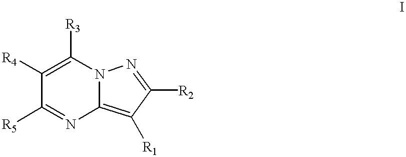
or a pharmaceutically acceptable salt, hydrate or prodrug thereof, wherein
R₁ is H, C₁₋₁₀ alkyl, C₃₋₆ cycloalkyl, aryl, halo, OH, C₃₋₁₀ heterocyclyl, or C₅₋₁₀ heteroaryl; said alkyl, aryl, heteroaryl and heterocyclyl being optionally substituted with from one to three members selected from R^{a};
R₂ and R₃ are independently H, C₁₋₆ alkyl, aryl, C₃₋₆ cycloalkyl, OH, NO₂, —NH₂, or halogen;
R₄ is H, C₁₋₁₀ alkyl, C₃₋₆ cycloalkyl, C₁₋₆ alkoxy C₂₋₁₀ alkenyl, C₂₋₁₀ alkynyl, aryl, C₃₋₁₀ heterocyclyl, C₁₋₆ alkoxyNR₇R₈, NO₂, OH, —NH₂ or C₅₋₁₀ heteroaryl, said alkyl, alkenyl, alkynyl, aryl, heteroaryl and heterocyclyl being optionally substituted with from one to three members selected from R^{a};
R₅ is H, or C₁₋₆ alkyl, OR, halo, NH₂ or NO₂;
R^{a} is H, C₁₋₁₀ alkyl, halogen, NO₂, OR, —NR, NR₇R₈, R₇R₈, aryl, C₅₋₁₀ heteroaryl or C₃₋₁₀ heterocyclyl,
R is H, or C₁₋₆ alkyl; and
R₇ and R₈ are independently H, C₁₋₁₀ alkyl, C₃₋₆ cycloalkyl, COR, COOR, COO—, aryl, C₃₋₁₀ heterocyclyl, or C₅₋₁₀ heteroaryl or NR₇R₈ can be taken together to form a heterocyclic 5-10 membered saturated or unsaturated ring containing, in addition to the nitrogen atom, one to two additional heteroatoms selected from the group consisting of N, O and S;
(b) a compound represented by formula (II): 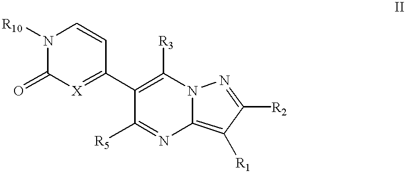
or a pharmaceutically acceptable salt, hydrate or prodrug thereof, wherein:
X is CH or N;
R₁ and R₃ are independently H, C₁₋₁₀ alkyl, C₃₋₆ cycloalkyl, aryl, halo, OH, C₃₋₁₀ heterocyclyl, or C₅₋₁₀ heteroaryl; said alkyl, aryl, heteroaryl and heterocyclyl being optionally substituted with from one to three members selected from R^{a};
R₂ is H, C₁₋₆ alkyl, aryl, C₃₋₆ cycloalkyl, OH, NO₂, —NH₂, or halogen;
R₁₀ is H, or C₁₋₆ alkyl, C₁₋₆ alkylR₉, NHC₁₋₆ alkylR₉, NR₇R₈, O—C₁₋₆ alkylR₉, aryl, C₃₋₁₀ heterocyclyl, said alkyl, aryl and heterocyclyl being optionally substituted with from one to three members selected from R^{a};
R₅ is H, C₁₋₆ alkyl, OH, O—C₁₋₆ alkyl, halo, NH₂ or NO₂;
R^{a} is H, C₁₋₁₀ alkyl, halogen, NO₂, OR, NR₇R₈, CN, aryl, C₅₋₁₀ heteroaryl or C₃₋₁₀ heterocyclyl,
R is H, or C₁₋₆ alkyl;
R₉ is aryl, C₃₋₁₀ heterocyclyl, or C₅₋₁₀ heteroaryl said aryl, heteroaryl and heterocyclyl being optionally substituted with from one to three members selected from R^{a}; and
R₇ and R₈ are independently H, C₁₋₁₀ alkyl, C₃₋₆ cycloalkyl, COR, COOR, COO—, aryl, C₃₋₁₀ heterocyclyl, or C₅₋₁₀ heteroaryl or NR₇R₈ can be taken together to form a heterocyclic 5-10 membered saturated or unsaturated ring containing, in addition to the nitrogen atom, one to two additional heteroatoms selected from the group consisting of N, O and S;
(c) a compound represented by formula (III): 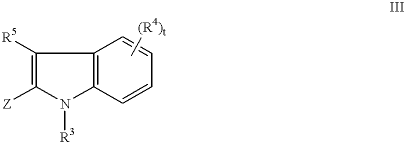
or a pharmaceutically acceptable salt, hydrate or prodrug thereof, wherein 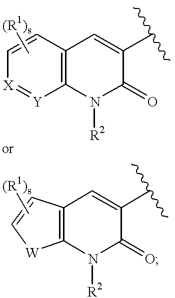
Z is
W is S or O;
a is 0 or 1;
b is 0 or 1;
s is 1 or 2;
t is 1, 2, or 3;
X═Y is C═N, N═C, or C═C;
R¹, R⁴ and R⁵ are independently selected from:
1) H,
2) (C═O)ₐO_{b}C₁-C₁₀ alkyl, optionally substituted with one to three substituents selected from R⁶,
3) (C═O)ₐO_{b}aryl, optionally substituted with one to three substituents selected from R⁶,
4) C₂-C₁₀ alkenyl, optionally substituted with one to three substituents selected from R⁶,
5) C₂-C₁₀ alkynyl, optionally substituted with one to three substituents selected from R⁶,
6) CO₂H,
7) halo,
8) OH,
9) O_{b}C₁-C₆ perfluoroalkyl, and
10) (C═O)ₐNR⁷R⁸;
R² and R³ are independently selected from the group consisting of:
1) H,
2) (C═O)OₐC₁-C₆ alkyl,
3) (C═O)Oₐaryl,
4) C₁-C₆ alkyl, and
5) aryl;
R⁶ is:
1) H,
2) (C═O)ₐO_{b}C₁-C₆ alkyl,
3) (C═O)ₐO_{b}aryl,
4) C₂-C₁₀ alkenyl,
5) C₂-C₁₋₁₀ alkynyl,
6) heterocyclyl,
7) CO₂H,
8) halo,
9) CN,
10) OH,
11) O_{b}C₁-C₆ perfluoroalkyl, or
12) NR⁷R⁸;
R^{6a} is:
1) H,
2) (C═O)ₐO_{b}C₁-C₆ alkyl,
3) (C═O)ₐO_{b}aryl,
4) C₂-C₁₀ alkenyl,
5) C₂-C₁₀ alkynyl,
6) heterocyclyl,
7) CO₂H,
8) halo,
9) CN,
10) OH,
11) O_{b}C₁-C₆ perfluoroalkyl, or
12) N(C₁-C₆ alkyl)₂;
R⁷ and R⁸ are independently selected from:
1) H,
2) (C═O)O_{b}C₁-C₁₀ alkyl, optionally substituted with one to three substituents selected from R^{6a},
3) (C═O)O_{b}aryl, optionally substituted with one to three substituents selected from R^{6a},
4) C₁-C₁₀ alkyl, optionally substituted with one to three substituents selected from R^{6a},
5) aryl, optionally substituted with one to three substituents selected from R^{6a},
6) C₂-C₁₀ alkenyl, optionally substituted with one to three substituents selected from R^{6a},
7) C₂-C₁₀ alkynyl, optionally substituted with one to three substituents selected from R^{6a}, and
8) heterocyclyl, or
R⁷ and R⁸ can be taken together with the nitrogen to which they are attached to form a 5-7 membered heterocycle containing, in addition to the nitrogen, one or two additional heteroatoms selected from N, O and S, said heterocycle optionally substituted with one to three substituents selected from R^{6a}.
(d) a compound represented by formula (IV): 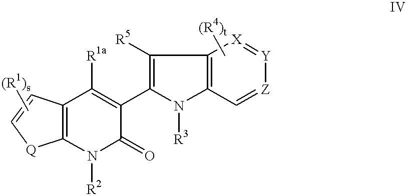
or a pharmaceutically acceptable salt or stereoisomer thereof, wherein
Q is S, O, or —E═D—;
X, Y and Z are C or N, so long as only one of X, Y and Z is N;
a is 0 or 1;
b is 0 or 1;
s is 1 or 2;
t is 1, 2, or 3;
m is 0, 1, or 2;
E═D is C═N, N═C, or C═C;
R¹, R^{1a}, R⁴ and R⁵ are independently selected from:
1) H,
2) (C═O)ₐO_{b}C₁-C₁₀ alkyl, optionally substituted with one to three substituents selected from R⁶,
3) (C═O)ₐO_{b}aryl, optionally substituted with one to three substituents selected from R⁶,
4) (C═O)ₐO_{b}C₂-C₁₀ alkenyl, optionally substituted with one to three substituents selected from R⁶,
5) (C═O)ₐO_{b}C₂-C₁₀ alkynyl, optionally substituted with one to three substituents selected from R⁶,
6) SOₘC₁-C₁₀ alkyl, optionally substituted with one to three substituents selected from R⁶,
7) SOₘaryl, optionally substituted with one to three substituents selected from R⁶,
8) CO₂H,
9) halo,
10) CN,
11) OH,
12) O_{b}C₁-C₆ perfluoroalkyl, and
13) (C═O)ₐNR⁷R⁸;
R² and R³ are independently selected from the group consisting of:
1) H,
2) (C═O)OₐC₁-C₁₀ alkyl,
3) (C═O)Oₐaryl,
4) C₁-C₁₀ alkyl,
5) SOₘC₁-C₁₀ alkyl,
6) SOₘaryl,
7) (C═O)ₐO_{b}C₂-C₁₀ alkenyl,
8) (C═O)ₐO_{b}C₂-C₁₀ alkynyl, and
9) aryl,
said alkyl, aryl, alkenyl and alkynyl is optionally substituted with one to three substituents selected from R⁶;
R⁶ is:
1) H,
2) (C═O)ₐO_{b}C₁-C₆ alkyl,
3) (C═O)ₐO_{b}aryl,
4) C₂-C₁₀ alkenyl,
5) C₂-C₁₀ alkynyl,
6) heterocyclyl,
7) CO₂H,
8) halo,
9) CN,
10) OH,
11) oxo,
12) O_{b}C₁-C₆ perfluoroalkyl, or
13) NR⁷R⁸;
R^{6a} is:
1) H,
2) (C═O)ₐO_{b}C₁-C₆ alkyl,
3) (C═O)ₐO_{b}aryl,
4) C₂-C₁₀ alkenyl,
5) C₂-C₁₀ alkynyl,
6) heterocyclyl,
7) CO₂H,
8) halo,
9) CN,
10) OH,
11) oxo,
12) O_{b}C₁-C₆ perfluoroalkyl, or
13) N(C₁-C₆ alkyl)₂;
R⁷ and R⁸ are independently selected from:
1) H,
2) (C═O)O_{b}C₁-C₁₀ alkyl, optionally substituted with one to three substituents selected from R^{6a},
3) (C═O)O_{b}aryl, optionally substituted with one to three substituents selected from R^{6a},
4) C₁-C₁₀ alkyl, optionally substituted with one to three substituents selected from R^{6a},
5) aryl, optionally substituted with one to three substituents selected from R^{6a},
6) C₂-C₁₀ alkenyl, optionally substituted with one to three substituents selected from R^{6a},
7) C₂-C₁₀ alkynyl, optionally substituted with one to three substituents selected from R^{6a}, and
8) heterocyclyl, or
R⁷ and R⁸ can be taken together with the nitrogen to which they are attached to form a 5-7 membered heterocycle containing, in addition to the nitrogen, one or two additional heteroatoms selected from N, O and S, said heterocycle optionally substituted with one to three substituents selected from R^{6a}.

17. The method according to claim 15 wherein the inhibitor of KDR is selected from:
4-(3-phenyl-pyrazolo[1,5-a]pyrimidin-6-yl)-1-(3-piperidin-1-yl-propyl)-1H-pyridin-2-one,
1-(2-morpholin-4-yl-ethyl)-4-(3-phenyl-pyrazolo[1,5-a]pyrimidin-6-yl)-1H-pyridin-2-one,
1-(3-dimethylamino-propyl)-4-(3-phenyl-pyrazolo[1,5-a]pyrimidin-6-yl)-1H-pyridin-2-one,
1-(1-methyl-piperidin-3-ylmethyl)-4-(3-phenyl-pyrazolo[1,5-a]pyrimidin-6-yl)-1H-pyridin-2-one,
1-[3-(4-methylpiperazin-1-yl)-propyl)]-4-(3-phenyl-pyrazolo[1,5-a]pyrimidin-6-yl)-1H-pyridin-2-one,
1-(2-dimethylamino-propyl)-4-(3-phenyl-pyrazolo[1,5-a]pyrimidin-6-yl)-1H-pyridin-2-one,
1-(1-dimethylamino-2-methyl-propyl)-4-(3-phenyl-pyrazolo[1,5-a]pyrimidin-6-yl)-1H-pyridin-2-one,
1-[2-(4-cyano-piperidin-1-yl-ethyl]-4-(3-phenyl-pyrazolo[1,5-a]pyrimidin-6-yl)-1H-pyridin-2-one,
1-(3-piperidin-1-yl-propyl)-4-(3-thiophen-3-yl-pyrazolo[1,5-a]pyrimidin-6-yl)-1H-pyridin-2-one,
1-(3-piperidin-1-yl-ethyl)-4-(3-thiophen-3-yl-pyrazolo[1,5-a]pyrimidin-6-yl)-1H-pyridin-2-one,
1-(2-morpholin-4-yl-ethyl)-4-(3-thiophen-3-yl-pyrazolo[1,5-a]pyrimidin-6-yl)-1H-pyridin-2-one,
1-(3-dimethylamino-propyl)-4-(3-thiophen-3-yl-pyrazolo[1,5-a]pyrimidin-6-yl)-1H-pyridin-2-one,
1-(1-methyl-piperidin-3-ylmethyl)-4-(3-thiophen-3-yl-pyrazolo[1,5-a]pyrimidin-6-yl)-1H-pyridin-2-one,
1-[3-(4-methylpiperazin-1-yl)-propyl)]-4-(3-thiophen-3-yl-pyrazolo[1,5-a]pyrimidin-6-yl)-1H-pyridin-2-one,
1-(2-dimethylamino-propyl)-4-(3-thiophen-3-yl-pyrazolo[1,5-a]pyrimidin-6-yl)-1H-pyridin-2-one,
1-(1-dimethylamino-2-methyl-propyl)-4-(3-thiophen-3-yl -pyrazolo[1,5-a]pyrimidin-6-yl)-1H-pyridin-2-one,
1-(3-dimethylamino-propyl)-4-(3-thiophen-3-yl-pyrazolo[1,5-a]pyrimidin-6-yl)-1H-pyridin-2-one,
1-[2-(4-cyano-piperidin-1-yl-ethyl]-4-(3-thiophen-3-yl-pyrazolo[1,5-a]pyrimidin-6-yl)-1H-pyridin-2-one,
4-(3-phenyl-pyrazolo[1,5-a]pyrimidin-6-yl)-1-(3-piperidin-1-yl-propyl)-1H-pyrimidin-2-one,
1-(2-morpholin-4-yl-ethyl)-4-(3-phenyl-pyrazolo[1,5-a]pyrimidin-6-yl)-1H-pyrimidin-2-one,
1-(3-dimethylamino-propyl)-4-(3-phenyl-pyrazolo[1,5-a]pyrimidin-6-yl)-1H-pyrimidin-2-one,
1-(1-methyl-piperidin-3-ylmethyl)-4-(3-phenyl-pyrazolo[1,5-a]pyrimidin-6-yl)-1H-pyrimidin-2-one,
1 1-[3-(4-methylpiperazin-1-yl)-propyl)]-4-(3-phenyl-pyrazolo[1,5-a]pyrimidin-6-yl)-1H-pyrimidin-2-one,
1-(2-dimethylamino-propyl)-4-(3-phenyl-pyrazolo[1,5-a]pyrimidin-6-yl)-1H-pyrimidin-2-one,
1-(1-dimethylamino-2-methyl-propyl)-4-(3-phenyl-pyrazolo[1,5-a]pyrimidin-6-yl)-1H-pyrimidin-2-one,
1-[2-(4-cyano-piperidin-1-yl-ethyl]-4-(3-phenyl-pyrazolo[1,5-a]pyrimidin-6-yl)-1H-pyrimidin-2-one
3-[5-(2-piperidin-1-yl-ethoxy)-1H-indol-2-yl]-1H-quinolin-2-one,
3-[5-(2-pyrrolidin-1-yl-ethoxy)-1H-indol-2-yl]-1H-quinolin-2-one,
3-[5-(2-morpholin-4-yl-ethoxy)-1H-indol-2-yl]-1H-quinolin-2-one,
3-[5-(3-dimethylamino-2-methyl-propoxy)-1H-indol-2-yl]-1H-quinolin-2-one,
3-[5-(3-piperidin-1-yl-propoxy)-1H-indol-2-yl]-1H-quinolin-2-one,
3-(5-{2-[benzyl-(2-methoxy-ethyl)-amino]-ethoxy}-1H-indol-2-yl)-1H-quinolin-2-one,
3-[5-(2-diethylamino-ethoxy)-1H-indol-2-yl]-1H-quinolin-2-one,
3-{5-[3-(benzyl-methyl-amino)-propoxy]-1H-indol-2-yl}-1H-quinolin-2-one,
1-{2-[2-(2-oxo-1,2-dihydro-quinolin-3-yl)-1H-indol-5-yloxy]-ethyl}-piperidine-4-carbonitrile,
3-{5-[3-(4-methyl-piperazin-1-yl)-propoxy]-1H-indol-2-yl}-1H-quinolin-2-one,
3-[5-(3-morpholin-4-yl-propoxy)-1H-indol-2-yl]-1H-quinolin-2-one,
3-(5-{2-[bis-(2-methoxy-ethyl)-amino]-ethoxy}-1H-indol-2-yl)-1H-quinolin-2-one,
3-(5-{2-[ethyl-(2-methoxy-ethyl)-amino]-ethoxy}-1H-indol-2-yl)-1H-quinolin-2-one,
3-(5-{2-[(2-methoxy-ethyl)-methyl-amino]-ethoxy}-1H-indol-2-yl)-1H-quinolin-2-one,
3-(1H-indol-2-yl)-1H-quinolin-2-one
3-(5-methoxy-1H-pyrrolo[3,2-b]pyridin-2-yl)-1H-quinolin-2-one;
3-(1H-pyrrolo[2,3-c]pyridin-2-yl)-1H-quinolin-2-one;
3-(1H-pyrrolo[3,2-c]pyridin-2-yl)-1H-quinolin-2-one;
3-(1H-pyrrolo[3,2-b]pyridin-2-yl)-1H-quinolin-2-one;
3-(5-methoxy-1H-pyrrolo[2,3-c]pyridin-2-yl)-1H-quinolin-2-one;
3-(5-oxo-4,5-dihydro-1H-pyrrolo[3,2-b]pyridin-2-yl)-1H-quinolin-2-one;
3-(5-oxo-5,6-dihydro-1H-pyrrolo[2,3-c]pyridin-2-yl)-1H-quinolin-2-one;
3-(4-oxo-4,5-dihydro-1H-pyrrolo[3,2-c]pyridin-2-yl)-1H-quinolin-2-one,
3-(4-fluorophenyl)-6-(4-pyridyl)pyrazolo(1,5-A)pyrimidine,
3-(3-chlorophenyl)-6-(4-pyridyl)pyrazolo(1,5-A)pyrimidine,
3-(3,4-methylenedioxypheny)-6-(4-pyridyl)pyrazolo(1,5-A)pyrimidine,
3-(phenyl)-6-(4-pyrimidyl)pyrazolo(1,5-A)pyrimidine,
3-(4-fluorophenyl)-6-(4-pyrimidyl)pyrazolo(1,5-A)pyrimidine,
3-(3-chlorophenyl)-6-(4-pyrimidyl)pyrazolo(1,5-A)pyrimidine,
3-(3-thienyl)-6-(4-pyrimidyl)pyrazolo(1,5-A)pyrimidine,
3-(3-acetamidophenyl)-6-(4-methylphenyl)pyrazolo(1,5-A)pyrimidine,
3-(3-thienyl)-6-(4-methylphenyl)pyrazolo(1,5-A)pyrimidine,
3-(phenyl)-6-(4-methoxyphenyl)pyrazolo(1,5-A)pyrimidine,
3-(3-acetamidophenyl)-6-(4-methoxyphenyl)pyrazolo(1,5-A)pyrimidine,
3-(3-thienyl)-6-(4-methoxyphenyl)pyrazolo(1,5-A)pyrimidine,
3-(phenyl)-6-(4-methoxyphenyl)pyrazolo(1,5-A)pyrimidine,
3-(4-pyridyl)-6-(4-methoxyphenyl)pyrazolo(1,5-A)pyrimidine,
3-(phenyl)-6-(4-chlorophenyl)pyrazolo(1,5-A)pyrimidine.
3-(4-pyridyl)-6-(4-chlorophenyl)pyrazolo(1,5-A)pyrimidine,
3-(phenyl)-6-(4-methylphenyl)pyrazolo(1,5-A)pyrimidine,
3-(4-pyridyl)-6-(4-methylphenyl)pyrazolo(1,5-A)pyrimidine,
3-(phenyl)-6-(2-pyridyl)pyrazolo(1,5-A)pyrimidine,
3-(4-pyridyl)-6-(2-pyridyl)pyrazolo(1,5-A)pyrimidine,
3-(phenyl)-6-(4-pyrimidyl)pyrazolo(1,5-A)pyrimidine,
3-(4-pyridyl)-6-(4-pyrimidyl)pyrazolo(1,5-A)pyrimidine,
3-(phenyl)-6-(2-pyrazinyl)pyrazolo(1,5-A)pyrimidine,
3-(4-pyridyl)-6-(2-pyrazinyl)pyrazolo(1,5-A)pyrimidine,
3-(3-pyridyl)-6-(4-methoxyphenyl)pyrazolo(1,5-A)pyrimidine,
3-(phenyl)-6-(4-pyridyl)pyrazolo(1,5-A)pyrimidine,
3-(3-pyridyl)-6-(4-pyridyl)pyrazolo(1,5-A)pyrimidine,
3-(4 pyridyl)-6-(4-methoxyphenyl)pyrazolo(1,5-A)pyrimidine,
3-(3-thienyl)-6-(4-methoxyphenyl)pyrazolo(1,5-A)pyrimidine,
3-(3-thienyl)-6-(4-hydroxyphenyl)pyrazolo(1,5-A)pyrimidine,
3-(3-thienyl)-6-(4-(2-(4-morpholinyl)ethoxy)phenyl)pyrazolo(1,5-A)pyrimidine,
3-(3-thienyl)-6-(cyclohexyl)pyrazolo(1,5-A)pyrimidine,
3-(bromo)-6-(4-methoxyphenyl)pyrazolo(1,5-A)pyrimidine,
3-(bromo)-6-(4-pyrimidyl)pyrazolo(1,5-A)pyrimidine,
3-(phenyl)-6-(2-(3-carboxy)pyridyl)pyrazolo(1,5-A)pyrimidine,
3-(3-thienyl)-6-(4-pyridyl)pyrazolo(1,5-A)pyrimidine,
or a pharmaceutically acceptable salt or optical isomer thereof.

18. A pharmaceutical composition for achieving a therapeutic effect in a mammal in need thereof which comprises amounts of at least one inhibitor of angiogenesis and at least one PSA conjugate.

19. The pharmaceutical composition according to claim 18 comprising an amount of an inhibitor of angiogenesis and an amount of a PSA conjugate.

20. The pharmaceutical composition according to claim 18 wherein the therapeutic effect is treatment of cancer.

21. The pharmaceutical composition according to claim 18 wherein the therapeutic effect is selected from inhibition of cancerous tumor growth and the regression of cancerous tumors.

22. The method according to claim 18 wherein the cancer is a cancer related to cells that express enzymatically active PSA.

23. The method according to claim 22 wherein the cancer is prostate cancer.

24. A method of preparing a pharmaceutical composition for achieving a therapeutic effect in a mammal in need thereof which comprises mixing amounts of at least one inhibitor of angiogenesis and at least one PSA conjugate.

25. The method of preparing a pharmaceutical composition according to claim 24 comprising mixing an amount of an angiogenesis inhibitor and an amount of an PSA conjugate.

26. A method of treating cancer in a mammal in need thereof which comprises administering to said mammal amounts of at least one inhibitor of angiogenesis and at least one PSA conjugate and applying to the mammal radiation therapy.

27. The method according to claim 26 wherein an amount of an angiogenesis inhibitor and an amount of a PSA conjugate are administered simultaneously.

28. The method according to claim 26 wherein an amount of an angiogenesis inhibitor and an amount of an PSA conjugate are administered consecutively.

29. A method for treating prostatic disease in a mammal in need thereof which comprises administering to said mammal amounts of at least one inhibitor of angiogenesis and at least one PSA conjugate.

30. The method according to claim 29 wherein the prostatic disease is selected from benign prostatic hyperplasia, prostatic intraepithelial neoplasia and prostate cancer.


SEQUENCE LISTING (54 sequences)

SEQ ID NO: 1 - protein, 7 aa, Artificial Sequence
  completely synthetic amino acid sequence
NKISYQS

SEQ ID NO: 2 - protein, 8 aa, Artificial Sequence
  completely synthetic amino acid sequence
NKISYQSS

SEQ ID NO: 3 - protein, 9 aa, Artificial Sequence
  completely synthetic amino acid sequence
NKISYQSSS

SEQ ID NO: 4 - protein, 10 aa, Artificial Sequence
  completely synthetic amino acid sequence
NKISYQSSST

SEQ ID NO: 5 - protein, 11 aa, Artificial Sequence
  completely synthetic amino acid sequence
NKISYQSSSTE

SEQ ID NO: 6 - protein, 12 aa, Artificial Sequence
  completely synthetic amino acid sequence
ANKISYQSSSTE

SEQ ID NO: 7 - protein, 11 aa, Artificial Sequence
  completely synthetic amino acid sequence
ANKISYQSSST

SEQ ID NO: 8 - protein, 12 aa, Artificial Sequence
  completely synthetic amino acid sequence
ANKISYQSSSTL

SEQ ID NO: 9 - protein, 12 aa, Artificial Sequence
  completely synthetic amino acid sequence
ANKASYQSASTL

SEQ ID NO: 10 - protein, 11 aa, Artificial Sequence
  completely synthetic amino acid sequence
ANKASYQSASL

SEQ ID NO: 11 - protein, 11 aa, Artificial Sequence
  completely synthetic amino acid sequence
ANKASYQSSSL

SEQ ID NO: 12 - protein, 10 aa, Artificial Sequence
  completely synthetic amino acid sequence
ANKASYQSSL

SEQ ID NO: 13 - protein, 7 aa, Artificial Sequence
  completely synthetic amino acid sequence
SYQSSSL

SEQ ID NO: 14 - protein, 7 aa, Artificial Sequence
  completely synthetic amino acid sequence
RYQSSSL

SEQ ID NO: 15 - protein, 7 aa, Artificial Sequence
  completely synthetic amino acid sequence
KYQSSSL

SEQ ID NO: 16 - protein, 6 aa, Artificial Sequence
  completely synthetic amino acid sequence
KYQSSL

SEQ ID NO: 17 - protein, 7 aa, Artificial Sequence
  completely synthetic amino acid sequence
KYQSSSL

SEQ ID NO: 18 - protein, 11 aa, Artificial Sequence
  completely synthetic amino acid sequence
LNKASYQSSSL

SEQ ID NO: 19 - protein, 7 aa, Artificial Sequence
  completely synthetic amino acid sequence
XSSXQSL

SEQ ID NO: 20 - protein, 6 aa, Artificial Sequence
  completely synthetic amino acid sequence
XSXQSL

SEQ ID NO: 21 - protein, 7 aa, Artificial Sequence
  completely synthetic amino acid sequence
XSSXQSL

SEQ ID NO: 22 - protein, 7 aa, Artificial Sequence
  completely synthetic amino acid sequence
XASXQSL

SEQ ID NO: 23 - protein, 7 aa, Artificial Sequence
  completely synthetic amino acid sequence
XASXQSL

SEQ ID NO: 24 - protein, 7 aa, Artificial Sequence
  completely synthetic amino acid sequence
PASXQSL

SEQ ID NO: 25 - protein, 7 aa, Artificial Sequence
  completely synthetic amino acid sequence
XASXQSL

SEQ ID NO: 26 - protein, 7 aa, Artificial Sequence
  completely synthetic amino acid sequence
XASXQSL

SEQ ID NO: 27 - protein, 7 aa, Artificial Sequence
  completely synthetic amino acid sequence
XASXQSL

SEQ ID NO: 28 - protein, 7 aa, Artificial Sequence
  completely synthetic amino acid sequence
XASXQSX

SEQ ID NO: 29 - protein, 7 aa, Artificial Sequence
  completely synthetic amino acid sequence
XASXQSL

SEQ ID NO: 30 - protein, 7 aa, Artificial Sequence
  completely synthetic amino acid sequence
XASXQSV

SEQ ID NO: 31 - protein, 7 aa, Artificial Sequence
  completely synthetic amino acid sequence
PASXQSL

SEQ ID NO: 32 - protein, 7 aa, Artificial Sequence
  completely synthetic amino acid sequence
SSSXQSV

SEQ ID NO: 33 - protein, 7 aa, Artificial Sequence
  completely synthetic amino acid sequence
XSSXQSV

SEQ ID NO: 34 - protein, 7 aa, Artificial Sequence
  completely synthetic amino acid sequence
SSSXQSL

SEQ ID NO: 35 - protein, 7 aa, Artificial Sequence
  completely synthetic amino acid sequence
XSSXQSL

SEQ ID NO: 36 - protein, 8 aa, Artificial Sequence
  completely synthetic amino acid sequence
XSSXQSSP

SEQ ID NO: 37 - protein, 7 aa, Artificial Sequence
  completely synthetic amino acid sequence
XSSXQSG

SEQ ID NO: 38 - protein, 8 aa, Artificial Sequence
  completely synthetic amino acid sequence
XSSXQSSG

SEQ ID NO: 39 - protein, 8 aa, Artificial Sequence
  completely synthetic amino acid sequence
XSSXQSSP

SEQ ID NO: 40 - protein, 7 aa, Artificial Sequence
  completely synthetic amino acid sequence
XSSXQSV

SEQ ID NO: 41 - protein, 8 aa, Artificial Sequence
  completely synthetic amino acid sequence
XSSXQSSP

SEQ ID NO: 42 - protein, 7 aa, Artificial Sequence
  completely synthetic amino acid sequence
XSSXQSP

SEQ ID NO: 43 - protein, 7 aa, Artificial Sequence
  completely synthetic amino acid sequence
XSSXQSP

SEQ ID NO: 44 - protein, 8 aa, Artificial Sequence
  completely synthetic amino acid sequence
XSSXQSSP

SEQ ID NO: 45 - protein, 7 aa, Artificial Sequence
  completely synthetic amino acid sequence
XSSXQSV

SEQ ID NO: 46 - protein, 7 aa, Artificial Sequence
  completely synthetic amino acid sequence
XSSXQSL

SEQ ID NO: 47 - protein, 5 aa, Artificial Sequence
  completely synthetic amino acid sequence
XSSXQ

SEQ ID NO: 48 - protein, 6 aa, Artificial Sequence
  completely synthetic amino acid sequence
XXQSSX

SEQ ID NO: 49 - protein, 5 aa, Artificial Sequence
  completely synthetic amino acid sequence
XXQSS

SEQ ID NO: 50 - protein, 6 aa, Artificial Sequence
  completely synthetic amino acid sequence
XSSXQX

SEQ ID NO: 51 - protein, 4 aa, Artificial Sequence
  completely synthetic amino acid sequence
XQSX

SEQ ID NO: 52 - protein, 4 aa, Artificial Sequence
  completely synthetic amino acid sequence
XQSX

SEQ ID NO: 53 - protein, 7 aa, Artificial Sequence
  completely synthetic amino acid sequence
XASXQSL

SEQ ID NO: 54 - protein, 7 aa, Artificial Sequence
  completely synthetic amino acid sequence
XASXQSX